(19) United States
(12) Patent Application Publication
(10) Pub. No.: US 2023/0233696 A1
(43) Pub. Date: Jul. 27, 2023

(54) TRIANTENNARY N-ACETYLGALACTOSAMINE MODIFIED HYDROXYL POLYAMIDOAMINE DENDRIMERS AND METHODS OF USE THEREOF

(71) Applicant: Ashvattha Therapeutics, Inc., Redwood City, CA (US)

(72) Inventors: Jeffrey Cleland, Redwood City, CA (US); Rishi Sharma, Redwood City, CA (US); Minghao Sun, Redwood City, CA (US)

(73) Assignee: Ashvattha Therapeutics, Inc., Redwood City, CA (US)

(21) Appl. No.: 17/782,596

(22) PCT Filed: Dec. 4, 2020
(86) PCT No.: PCT/US2020/063342

§ 371 Date: Jun. 3, 2022

Related U.S. Application Data
(60) Provisional application No. 63/108,186, filed on Oct. 30, 2020.
(60) Provisional application No. 63/086,109, filed on Oct. 1, 2020.
(60) Provisional application No. 63/067,155, filed on Aug. 18, 2020.
(60) Provisional application No. 62/943,705, filed on Dec. 4, 2019.

(51) Int. Cl. A61K 47/61; A61P 1/16; A61K 45/06

(52) U.S. Cl. CPC A61K 47/61; A61K 45/06; A61P 1/16

(57) ABSTRACT

It has been established that dendrimers conjugated or complexed with the carbohydrate triantennary N-Acetylgalactosamine (triantennary-β-GalNAc) selectively accumulate within hepatocyte cells and selectively deliver therapeutic, prophylactic or diagnostic agents to the liver. Compositions of dendrimers complexed with triantennary-β-GalNAc and one or more agents to prevent, treat or diagnose a liver injury, liver disease or liver disorder in a subject in need thereof, and methods of use thereof, have been developed. The compositions are particularly suited for treating and/or ameliorating one or more symptoms of nonalcoholic fatty liver disease (NAFLD) and liver cancer, with decreased toxicity.

### CROSS-REFERENCE TO RELATED APPLICATIONS

This application is a national stage filing under 35 U.S.C. § 371 of International Patent Application Serial No. PCT/US2020/063342, filed Dec. 4, 2020, which claims the benefit of U.S. Provisional Application No. 62/943,705, filed Dec. 4, 2019, U.S. Provisional Application No. 63/067,155, filed Aug. 18, 2020, U.S. Provisional Application No. 63/086,109, filed Oct. 1, 2020, and U.S. Provisional Application No. 63/108,186, filed Oct. 30, 2020, which are incorporated by reference in their entirety.

### FIELD OF THE INVENTION

The invention is generally in the field of drug delivery, and in particular, a method of delivering drugs bound to dendrimers, which selectively target sites or regions of the liver.

### BACKGROUND OF THE INVENTION

Liver diseases, such as liver infections, liver cirrhosis, drug-induced liver failure and hepato-cellular carcinoma, continue to pose a significant health challenge worldwide. The prevalence of liver diseases is increasing worldwide, with an estimated 844 million people around the globe suffering from a chronic liver problem, and around 2 million deaths from liver disorders each year.

Non-alcoholic fatty liver disease (NAFLD), also known as non-alcoholic steatohepatitis (NASH) is currently the most common liver disorder, and is predicted to be the most frequent indication for liver transplantation by 2030. NAFLD/NASH can result in liver cirrhosis (scarring) or liver cancer, and is associated with significant morbidity and mortality. The global estimated prevalence of NAFLD is from 6.3% to 33% in the general population, with a median prevalence of 20%. The median prevalence of NAFLD is generally higher in developed countries.

Despite these alarming numbers, the current treatment options for many hepatic diseases are limited, and lack the necessary efficacy in treating advanced and severe cases. While much progress has been made in elucidating the epidemiology, natural history, and pathogenesis of NAFLD/NASH, there remains no effective therapy, with limited options of evidence-based clinical guidelines for patient management. Pharmacological treatment of patients with NAFLD is still evolving, with no single therapy that has clearly been proven effective, especially, in modifying the course of the disease.

Hepatocytes are the most abundant liver cell type, constitute >80% of the liver biomass and are predominantly implicated in most liver disorders, such as hepatocellular carcinoma, drug-induced liver failure, hepatitis, and non-alcoholic steatohepatitis. Effectively delivering drugs to treat diseased hepatocytes represents a challenge. When injected, most drugs will accumulate in the liver, but tend to be cleared through macrophages and Kupffer cells rather than hepatocytes, such that selectively and effectively targeting hepatocytes is difficult.

Therefore, it is an object of the invention to provide compositions and methods for selectively reducing or preventing one or more symptoms of liver diseases and/or disorders.

It is also an object of the invention to provide compositions that reduce or prevent the pathological processes associated with the development and progression of liver diseases and/or disorders, and methods of making and using thereof.

It is yet another object of the invention to provide compositions and methods for selectively targeting active agents to hepatocytes at the site in need thereof.

### SUMMARY OF THE INVENTION

It has been established that dendrimers complexed or conjugated with triantennary-N-Acetylgalactosamine (GalNAc) selectively deliver active agents to hepatocytes in vivo. In some embodiments, the dendrimers are covalently conjugated to triantennary-N-acetylgalactosamine (GalNAc) via an ester, ether, or amide bonds, optionally with one or more linkers.

Compositions and methods for treating or preventing one or more symptoms of a liver disease and/or disorder in a subject in need thereof are provided. Typically, methods for treating liver disease in a subject include administering to the subject a formulation including triantennary-GalNAc modified dendrimers complexed to, covalently conjugated to, or having intra-molecularly dispersed or encapsulated therein one or more therapeutic or prophylactic agents. The formulation is typically administered in an amount effective to treat, alleviate or prevent one or more symptoms of the liver disease and/or disorder in the recipient. Exemplary liver diseases and/or disorders that can be treated include nonalcoholic fatty liver disease (NAFLD), non-alcoholic steatohepatitis (NASH), drug-induced liver failure, hepatitis, liver fibrosis, liver cirrhosis, hepatocellular carcinoma, or combinations thereof. In some embodiments, the dendrimers are hydroxyl-terminated dendrimers. In some embodiments, the dendrimers are generation 4, generation 5, generation 6, generation 7, or generation 8 poly(amidoamine) (PAMAM) dendrimers.

The methods are used to selectively deliver one or more active agents, such as therapeutic, prophylactic and/or diagnostic agents, into the hepatocyte cells of the recipient. Exemplary therapeutic agents that can be delivered include angiotensin II receptor blockers, Farnesoid X receptor agonists, death receptor 5 agonists, sodium-glucose cotransporter type-2 (SGLT2) inhibitors, lysophosphatidic acid (LPA) 1 receptor antagonists, endothelin-A receptor antagonist, PPARδ agonists, AT1 receptor antagonists, CCR5/CCR2 antagonists, anti-fibrotic agents, anti-inflammatory agents, and/or anti-oxidant agents. In some embodiments, the angiotensin II receptor blocker is telmisartan, or a telmisartan-amide derivative, or a telmisartan-ester derivative. In some embodiments, the FXR agonist is chenodeoxycholic acid, or a chenodeoxycholic acid-amide derivative, or a chenodeoxycholic acid-ester derivative. Exemplary SGLT2 inhibitors that can be delivered include Phlorizin, T-1095, canagliflozin, dapagliflozin, ipragliflozin, tofogliflozin, empagliflozin, luseogliflozin, ertugliflozin, remogliflozin etabonate, or a derivative thereof. In some embodiments, the PPARδ agonist is GW0742, a GW0742-amide derivative and a GW0742-ester derivative. In some embodiments, the anti-oxidant agent is vitamin E, or a derivative thereof.

Typically, the methods deliver active agents to the subject in an amount effective to achieve a desired physiological response in the subject. For example, in some embodiments the methods deliver active agents to the subject in an amount effective to reduce serum levels of one or more of alanine aminotransferase (ALT), aspartate aminotransferase (AST), triglyceride (TG), gamma-glutamyltrasferase (GGT), total cholesterol (TC), low density lipoprotein (LDP), fasting blood sugar, or combinations thereof in the subject. In some embodiments, the methods deliver active agents to the subject in an amount effective to reduce one or more of steatosis, inflammation, ballooning, fibrosis, cirrhosis, or combinations thereof in the subject. In some embodiments, the methods deliver active agents to the subject in an amount effective to reduce lobular inflammation in the liver; to reduce the amount or presence of one or more pro-inflammatory cells, chemokines, and/or cytokines in the liver; or to reduce one or more pro-inflammatory cytokines including TNF-α, IL-6, and IL-1α.

In some embodiments, therapeutic, prophylactic and/or diagnostic agents to be delivered into the hepatocyte cells include STING agonists, CSF1R inhibitors, PARP inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, chemotherapeutics, and cytotoxic agents. In some embodiments, the STING agonist is a cyclic dinucleotide GMP-AMP or DMXAA. In some embodiments, the CSF1R inhibitor is selected from the group consisting of PLX3397, PLX108-01, ARRY-382, PLX7486, BLZ945, JNJ-40346527, and GW 2580. In some embodiments, the PARP inhibitor is selected from the group consisting of Olaparib, Veliparib, Niraparib, and Rucaparib. In some embodiments, VEGFR tyrosine kinase inhibitor is selected from the group consisting of sunitinib or a derivative or analog thereof, sorafenib, pazopanib, vandetanib, axitinib, cediranib, vatalanib, dasatinib, nintedanib, and motesanib. In some embodiments, the MEK inhibitor is selected from the group consisting of Trametinib, Cobimetinib, Binimetinib, Selumetinib, PD325901, PD035901, PD032901, and TAK-733. In some embodiments, glutaminase inhibitor is selected from the group consisting of Bis-2-(5-phenylacetimido-1,2,4-thiadiazol-2-yl)ethyl sulfide (BPTES) and 6-diazo-5-oxo-L-norleucine (DON), azaserine, acivicin, and CB-839. In some embodiments, CXCR2 inhibitor is Navarixin, SB225002, or SB332235. In some embodiments, CD73 inhibitor is APCP, quercetin, or tenofovir, or a derivative, analogue thereof. In some embodiments, the arginase inhibitor is a derivative or analogue of 2-(S)-amino-6-boronohexanoic acid. In some embodiments, the PI3K inhibitor is selected from the group consisting of alpelisib, serabelisib, pilaralisib, WX-037, dactolisib, prexasertib, voxtalisib, PX-866, ZSTK474, buparlisib, pictilisib, and copanlisib. In some embodiments, immunomodulatory agent is a SHP2 inhibitor. In some embodiments, the cytotoxic agent is Auristatin E or Mertansine. In some embodiments, the chemotherapeutic agent is selected from the groups consisting of amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, daunorubicin, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab, cetuximab, rituximab, and bevacizumab. In preferred embodiments, the methods deliver active agents to the subject in an amount effective to reduce tumor size, and/or effective to enhance tumor-specific cytotoxic T cell responses in the subject.

In some embodiments, therapeutic, prophylactic and/or diagnostic agents are covalently conjugated to the dendrimer, optionally via a linker or spacer moiety, via a linkage selected from the group consisting of an ether, ester, and amide linkage. In preferred embodiments, therapeutic, prophylactic and/or diagnostic agents are covalently conjugated to the dendrimer, optionally via a linker or spacer moiety, via an amide or ether linkage.

In some embodiments, the formulation is formulated for intravenous, subcutaneous, or intramuscular administration to the subject, or for enteral administration. In some embodiments, the formulation is administered prior to, in conjunction with, subsequent to, or in alternation with treatment with one or more additional therapies or procedures. Exemplary additional procedures include administering one or more therapeutic, prophylactic and/or diagnostic agents to prevent or treat one or more symptoms of associated diseases or conditions of liver injuries such as infections, sepsis, diabetic complications, hypertension, obesity, high blood pressure, heart failure, kidney diseases, and cancers.

Pharmaceutical formulations of the triantennary-GalNAc modified dendrimers complexed to, covalently conjugated to, or having intra-molecularly dispersed or encapsulated therein one or more therapeutic or prophylactic agents are also described. Kits including the triantennary-GalNAc modified dendrimers complexed to, covalently conjugated to, or having intra-molecularly dispersed or encapsulated therein one or more therapeutic or prophylactic agents are also provided.

Methods of making triantennary-GalNAc modified dendrimers, and methods of making making triantennary-GalNAc modified dendrimers complexed to, covalently conjugated to, or having intra-molecularly dispersed or encapsulated therein one or more therapeutic or prophylactic agents are also described.

### BRIEF DESCRIPTION OF THE DRAWINGS

FIG. 1 is a scheme showing synthesis of P-GalNAc-triantennary-PEG3-Azide (AB3 building block). Reagents and conditions are indicated as follows: (i) scandium triflate, DCE, 3h, 80° C., (ii) propargyl bromide, toluene, sodium hydroxide, water, TBAB, (iii) pyridine, thionyl chloride, chloroform, 65° C., 2h; (iv) tetrabutylammonium hydrogen sulfate, 50% NaOH, 16h, rt; (v) (iii) CuSO₄.5H₂O, Na ascorbate, THF, water, 10 h; (vi) DMF, tetrabutylammonium iodide, NaN₃, 80° C., 5h; (vii) sodium methoxide, dry methanol, 30° C., 3h.

FIG. 2 is a scheme showing synthesis of Dendrimer-triantennary-β-GalNAc-CY5. Reagents and conditions are indicated as follows: (i) EDC, DMAP, DMF, RT, 24 h; (ii) CuSO₄.5H₂O, Na ascorbate, DMF, water, 8h; (iii) CuSO₄.5H₂O, Na ascorbate, DMF, water, 8h.

FIG. 3 is a scheme showing synthesis of Telmisartan ester conjugate to dendrimer-Triantenary-β-GlcNAc. Reagents and conditions are indicated as follows: (i) DCC, DMAP, DCM, RT, 4 h; (ii) EDC, DMAP, DMF, RT, 24 h; (iii) CuSO₄.5H₂O, Na ascorbate, DMF, water, 8h; (iv) CuSO₄.5H₂O, Na ascorbate, DMF, water, 8h.

FIG. 4 is a scheme showing synthesis of Telmisartan amide conjugate to dendrimer-Triantenary-β-GlcNAc. Reagents and conditions are indicated as follows: (i) HATU, DIPEA, DCM, RT, 4 h; (ii) EDC, DMAP, DMF, RT, 24 h; (iii) CuSO₄.5H₂O, Na ascorbate, DMF, water, 8h; (iv) CuSO₄.5H₂O, Na ascorbate, DMF, water, 8h.

FIG. 5 is a bar graph showing in vitro drug release from Dendrimer-telmisartan ester conjugate over a period of 18 days in plasma (pH 7.4, PBS) and intracellular conditions (pH 5.5, esterase).

FIG. 6 is a bar graph showing in vitro drug release from Dendrimer-telmisartan amide conjugate over a period of 18 days in plasma (pH 7.4, PBS) and intracellular conditions (pH 5.5, esterase).

FIG. 7 is a bar graph showing in vitro drug release from Dendrimer-telmisartan amide conjugate over a period of 48 hours in human, mouse and rat plasma at 37° C.

FIG. 8 is a scheme showing synthesis of Dendrimer-Triantenary-β-GlcNAc-azide-Obeticholic acid conjugate. Reagents and conditions are indicated as follows: (i) EDC, DMAP, DCM, RT, 4 h; (ii) CuSO₄-5H₂O, Na ascorbate, DMF, water, 8h; (iii) CuSO₄-5H₂O, Na ascorbate, DMF, water, 8h.

FIGS. 9A-9C are plots showing body weight in grams (FIG. 9A), liver weight in grams (FIG. 9B), and liver-to-body weight ratio (FIG. 9C) in Normal mice, and non-alcoholic Steatohepatitis (NASH) mice treated with vehicle, free Telmisartan, obeticholic acid (OCA), high-dose dendrimer-triantenary-β-GlcNAc-azide-Telmisartan amide conjugate (D-Tel high), low-dose D-Tel (D-Tel low), high-dose dendrimer-triantenary-β-GlcNAc-azide-Telmisartan ester conjugate (D-TelB high), high-dose dendrimer-triantenary-β-GlcNAc-azide-obeticholic acid ester conjugate (D-OCA high), and low-dose D-OCA (D-OCA low), when sacrificed at 9 weeks of age. * p<0.05; *** p<0.001; ****p<0.0001 vs Vehicle.

FIGS. 10A and 10B are plots showing levels of aminotransferase (ALT) in the serum (FIG. 10A) and levels of triglyceride in the liver (FIG. 10B) of Normal mice, and NASH mice treated with vehicle, free Telmisartan, OCA, D-Tel high, D-Tel low, D-TelB high, D-OCA high, and D-OCA low, when sacrificed at 9 weeks of age. * p<0.05; ** p<0.01; ****p<0.0001 vs Vehicle.

FIGS. 11A-11D are plots showing nonalcoholic fatty liver disease (NAFLD) activity score (FIG. 11A), steatosis score (FIG. 11B), inflammation score (FIG. 11C), and ballooning score (FIG. 11D) in the livers of Normal mice, and NASH mice treated with vehicle, free Telmisartan, OCA, D-Tel high, D-Tel low, D-TelB high, D-OCA high, and D-OCA low, when sacrificed at 9 weeks of age. * p<0.05; ** p<0.01; ****p<0.0001 vs Vehicle.

FIG. 12 is a plot showing Sirius red-positive area in the livers of normal mice, and NASH mice treated with vehicle, free Telmisartan, OCA, D-Tel high, D-Tel low, D-TelB high, D-OCA high, and D-OCA low, when sacrificed at 9 weeks of age. * p<0.05; ** p<0.01; ****p<0.0001 vs Vehicle.

FIG. 13A is a synthesis scheme of dendrimer conjugated to two different classes of active agents R1 and R2; FIG. 13B shows exemplary R1 groups, including capecitabine and gemcitabine, and analogs thereof, and FIG. 13C shows exemplary R2 groups such as TIE II inhibitors and analogs thereof.

FIGS. 14A and 14B are synthesis schemes of dendrimer conjugated to two exemplary TLR4 agonists.

FIG. 15 is a synthesis scheme of dendrimer conjugated to an exemplary CSF1R inhibitor.

FIG. 16 is a synthesis scheme for Dendrimer-N-Acetyl-L-cysteine methyl ester conjugate.

FIGS. 17A and 17B are schemes showing chemical reaction steps for the synthesis of a dendrimer-GW 2580 ether conjugate (FIG. 17A) and a dendrimer-GW 2580 ester conjugate (FIG. 17B), respectively.

### DETAILED DESCRIPTION OF THE INVENTION

### I. Definitions

The terms “active agent” or “biologically active agent” refer to therapeutic, prophylactic, or diagnostic agents, and are used interchangeably to refer to a chemical or biological compound that induces a desired pharmacological and/or physiological effect, which may be prophylactic, therapeutic or diagnostic. These may be a nucleic acid, a nucleic acid analog, a small molecule having a molecular weight less than 2 kDa, more typically less than 1 kDa, a peptidomimetic, a protein or peptide, carbohydrate or sugar, lipid, or surfactant, or a combination thereof. The terms also encompass pharmaceutically acceptable, pharmacologically active derivatives of active agents, including, but not limited to, salts, esters, amides, prodrugs, active metabolites, and analogs.

The term “therapeutic agent” refers to an active agent that can be administered to treat one or more symptoms of a disease or disorder.

The term “diagnostic agent” refers to an active agent that can be administered to reveal, pinpoint, and define the localization of a pathological process. The diagnostic agents can label target cells that allow subsequent detection or imaging of these labeled target cells.

The term “prophylactic agent” refers to an active agent that can be administered to prevent disease or to prevent certain conditions or symptoms.

The term “prodrug”, refers to a pharmacological substance (drug) that is administered to a subject in an inactive (or significantly less active) form. Once administered, the prodrug is metabolized in the body (in vivo) by enzymatic or chemical reactions, or by a combination of the two, into a compound having the desired pharmacological activity. Prodrugs can be prepared by replacing appropriate functionalities present in the compounds described above with “pro-moieties” as described, for example, in H. Bundgaar, Design of Prodrugs (1985). For further discussion of prodrugs, see, for example, Rautio, J. et al. Nature Reviews Drug Discovery. 7:255-270 (2008).

The terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The terms “immunomodulatory agent” or “immunotherapeutic agent” refer to an active agent that can be administered to regulate, enhance, reduce, prolong, decrease or otherwise alter one or more factors of the innate or adaptive immune response in the recipient. Generally, immunomodulatory agents can modulate immune microenvironment for a desired immunological response by targeting one or more immune cells or cell types at a target site, and thus, are not necessarily specific to any cancer type. For example, the blockade of a single molecule, programmed cell-death protein 1 (PD-1) on immune cells, has resulted in anti-tumor activity. In some embodiments, the immunomodulatory agents are specifically delivered to inhibit or reduce suppressive immune cells such as tumor associated macrophages for an enhanced anti-tumor response at a tumor site.

The term “immunosuppressive cells” refer to immune cells that promote tumor growth, angiogenesis, invasion, metastasis, resistance to therapy, or a combination thereof. Exemplary immunosuppressive cells including cancer-associated fibroblasts, myeloid-derived suppressor cells (MDSCs), regulatory T cells (Treg), mesenchymal stromal cells (MSCs) and TIE2-expressing monocytes, and tumor-associated macrophages (TAMs).

The term “pro-inflammatory cells” refer to immune cells that promote pro-inflammatory activities, secretion of pro-inflammatory cytokines such as IL-12, IFN-γ, and TNF-α, or a combination thereof. Exemplary pro-inflammatory cells including pro-inflammatory M1 macrophages or classically activated macrophages (CAMs).

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a prophylactic agent or therapeutic agent to reduce or diminish the risk of developing a liver disease/disorder or to reduce or diminish one or more symptoms of a liver disease/disorder, such as reducing inflammation in the liver. Additional desired results also include reducing and/or inhibiting serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality. In the case of cancer or tumor, an effective amount of the drug may have the effect of reducing the number of cancer cells; reducing the tumor size; inhibiting cancer cell infiltration into peripheral organs; inhibiting tumor metastasis; inhibiting tumor growth; and/or relieving one or more of the symptoms associated with the disorder. An effective amount can be administered in one or more administrations.

The terms “inhibit” or “reduce” mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. For example, dendrimer compositions including one or more agents may inhibit or reduce serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality in a diseased liver by about 10%, 20%, 30%, 40%, 50%, 75%, 85%, 90%, 95%, or 99% from subjects that did not receive, or were not treated, or prior to treatment with the dendrimer compositions. In some embodiments, the inhibition and reduction are compared at mRNAs, proteins, cells, tissues and organs levels. For example, an inhibition and reduction in tumor proliferation, or tumor size/volume.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated with liver diseases/disorders are mitigated or eliminated, including, but not limited to, reducing and/or inhibiting elevations of the transaminases including alanine transaminase (ALT) and aspartate transaminase (AST), reducing the proliferation of cancerous cells in the case of liver cancer, increasing the quality of life of those suffering from the disease, decreasing the dose of other medications required to treat the disease, delaying the progression of the disease, and/or prolonging survival of individuals.

The phrase “enhancing T-cell function” means to induce, cause or stimulate a T-cell to have a sustained or amplified biological function, or renew or reactivate exhausted or inactive T-cells. Examples of enhancing T-cell function include: increased secretion of Granzyme B, and/or IFN-γ from CD8+ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, or 200%. The manner of measuring this enhancement is known to one of ordinary skill in the art.

The term “tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The term “immunogenicity” refers to the ability of a particular substance to provoke an immune response. Tumors can be immunogenic and enhancing tumor immunogenicity aids in the clearance of the tumor cells by the immune response.

The term “biodegradable” refers to a material that will degrade or erode under physiological conditions to smaller units or chemical species that are capable of being metabolized, eliminated, or excreted by the body. The degradation time of a material is a function of composition and morphology of the material.

The term “dendrimer” includes, but is not limited to, a molecular architecture with an interior core, interior layers (or “generations”) of repeating units regularly attached to the interior core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type or cell activation state, or a subcellular compartment. In some embodiments, the targeting moiety directs the localization of an active agent.

The term “prolonged residence time” refers to an increase in the time required for an agent to be cleared from a patient’s body, or organ or tissue of that patient relative to a standard of comparison, such as a comparable agent without conjugation to a delivery vehicle such as a dendrimer. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, or 10000 times longer than a standard of comparison such as a comparable agent without a dendrimer that specifically target specific cell types associated with the site of inflammation and/or a tumor region.

The terms “incorporated” and “encapsulated” refer to incorporating, formulating, or otherwise including an active agent into and/or onto a composition. For example, an active agent or other material can be incorporated into a dendrimer, including to one or more surface functional groups of such dendrimer (by covalent, ionic, or other binding interaction), physical admixture, enveloping the agent within the dendritic structure, encapsulated inside the dendritic structure, etc.

The term “neutral surface charge” of a particle refers to the electrokinetic potential (zeta-potential) of a particle that is 0 mV. In some embodiments, the term “near-neutral surface charge” refers to a zeta-potential that is approximately 0 mV, such as from -10 mV to 10 mV, from -5 mV to 5 mV, preferably from -1 mV to 1 mV.

### II. Compositions

It has been established that dendrimers conjugated or complexed with the carbohydrate triantennary N-Acetylgalactosamine (GalNAc) selectively accumulate within hepatocyte cells and prevent and/or treat liver diseases.

Compositions of dendrimers complexed with carbohydrates suitable for delivering one or more active agents, particularly one or more active agents to prevent, treat or diagnose a liver injury, liver disease, or liver disorder in a subject in need thereof, have been developed. The compositions are particularly suited for treating and/or ameliorating one or more symptoms of nonalcoholic fatty liver disease (NAFLD) and/or hepatocellular carcinoma (HCC).

Compositions of dendrimer-carbohydrate complexes including one or more prophylactic, therapeutic, and/or diagnostic agents encapsulated, associated, and/or conjugated in the dendrimers are provided. Generally, one or more active agent is encapsulated, associated, and/or conjugated in the dendrimer complex at a concentration of about 0.01% weight to weight (w/w) to about 30% w/w, about 1% w/w to about 25% w/w, about 5% w/w to about 20% w/w, and about 10% w/w to about 15% w/w. Preferably, an active agent is covalently conjugated to the dendrimer via one or more linkages such as disulfide, ester, ether, thioester, carbamate, carbonate, hydrazine, and amide, optionally via one or more spacers. In some embodiments, the spacer is an active agent, such as telmisartan. Exemplary active agents include angiotensin II receptor blockers, FXR agonists, PPARδ agonists, anti-oxidants, anti-inflammatory drugs, chemotherapeutics, anti-fibrotic drugs, and anti-infective agents.

The presence of the additional agents can affect the zeta-potential, or the surface charge of the dendrimer. In one embodiment, the zeta potential of the dendrimer-carbohydrate complexes is between -100 mV and 100 mV, between -50 mV and 50 mV, between -25 mV and 25 mV, between -20 mV and 20 mV, between -10 mV and 10 mV, between -10 mV and 5 mV, between -5 mV and 5 mV, or between -2 mV and 2 mV. In a preferred embodiment, the surface charge is neutral or near neutral. The range above is inclusive of all values from -100 mV to 100 mV.

### A. Dendrimers

Dendrimers are three-dimensional, hyperbranched, monodispersed, globular and polyvalent macromolecules including a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nano-carriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Recent studies have shown that dendrimer surface groups have a significant impact on their biodistribution (Nance, E., et al., Biomaterials, 101, 96 (2016)). Hydroxyl terminated generation 4 PAMAM dendrimers (~4 nm size) without any targeting ligand cross the impaired BBB upon systemic administration in a rabbit model of cerebral palsy (CP) significantly more (> 20 fold) as compared to healthy controls, and selectively target activated microglia and astrocytes (Lesniak, W. G., et al., Mol Pharm, 10 (2013)).

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, dendrimers have a diameter between about 1 nm and about 50 nm, more typically between about 1 nm and about 20 nm, between about 1 nm and about 10 nm, or between about 1 nm and about 5 nm. In some embodiments, the diameter is between about 1 nm and about 2 nm. Conjugates are generally in the same size range, although large proteins such as antibodies may increase the size by 5-15 nm. In general, agent is encapsulated in a ratio of agent to dendrimer of between 1:1 and 4:1 for the larger generation dendrimers. In preferred embodiments, the dendrimers have a diameter effective to target hepatocytes and to retain in hepatocytes for a prolonged period.

In some embodiments, dendrimers have a molecular weight between about 500 Daltons and about 100,000 Daltons, preferably between about 500 Daltons and about 50,000 Daltons, most preferably between about 1,000 Daltons and about 20,000 Dalton.

Suitable dendrimers scaffolds that can be used include poly(amidoamine), also known as PAMAM, or STARBURST™ dendrimers; polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means a poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In the preferred embodiment, the dendrimers are soluble in the formulation and are generation (“G”) 4, 5 or 6 dendrimers (i.e., G4-G6 dendrimers), and/or G4-G10 dendrimers, G6-G10 dendrimers, or G2-G10 dendrimers. The dendrimers may have hydroxyl groups attached to their functional surface groups. In preferred embodiments, the dendrimers are generation 4, generation 5, generation 6, generation 7, or generation 8 hydroxyl terminated polyamidoamine dendrimers.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties such as triantennary-N-Acetylgalactosamine (GalNAc) and active agents, directly or indirectly through a linker.

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO 2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

In some embodiments, the dendrimer specifically targets a particular tissue region and/or cell type, e.g., hepatocytes, tumor associated macrophages (TAMs) in tumor/cancer in the liver, or pro-inflammatory macrophages involved in autoimmune diseases in the liver.

In preferred embodiments, the dendrimers have a plurality of hydroxyl (—OH) groups on the periphery of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50 surface hydroxyl groups/surface area in nm². In further embodiments, the surface density of hydroxyl (-OH) groups is between about 1 and about 50, preferably 5-20 surface hydroxyl group/nm² (number of hydroxyl surface groups/surface area in nm²) while having a molecular weight of between about 500 Da and about 10 kDa. In some embodiments, the percentage of free, i.e., un-conjugated hydroxyl groups out of total surface groups (conjugated and un-conjugated) on the dendrimer is more than 70%, 75%, 80%, 85%, 90%, 95%, and/or less than 100%.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50 hydroxyl groups/nm³. In some embodiments, the volumetric density of hydroxyl groups is between about 4 and about 50 hydroxyl groups/nm³, preferably between about 5 and about 30 groups/nm³, more preferably between about 10 and about 20 hydroxyl groups/nm³.

### B. Dendrimer Modified With Triantennary N-Acetylgalactosamine (GalNAc)

It has been established that dendrimers conjugated or complexed with the carbohydrate triantennary N-Acetylgalactosamine (GalNAc) selectively accumulate within hepatocyte cells. Compositions of dendrimers modified by addition of triantennary N-Acetylgalactosamine (GalNAc) to the dendrimer surface are described.

The abundantly expressed asialoglycoprotein receptor (ASGPR) on hepatocytes can selectively recognize galactose and N-acetylgalactosamine (GalNAc) through carbohydrate recognition domain (CRD) and binds to the receptor tightly. The efficient binding of carbohydrate moieties to the ASGPR receptors allows selective internalization within the hepatocyte via receptor-mediated endocytosis. The low pH in the endosomes results in the disruption of the tetravalent calcium-chelation between the sugar ligand and the ASGPR receptor, which releases the ligand in the hepatocytes. Once the ligand is released, the receptor complex recycles allowing large amounts of ligand to be internalized into hepatocytes without saturation effects. GalNAc binding to ASGPR occurs at the sinusoidal surface of the hepatocyte, which contains ~500,000 ASGPR receptors per cell, of which about 5%-10% are present at the cell surface at any one time. Previous studies have shown that the binding of ligands to ASGPR is dependent upon the type of sugar (GalNAc > Gal) and number of sugars with 4= 3 > 2 > 1. X-ray crystal structures of the extracellular domain of ASGPR revealed a shallow carbohydrate-binding pocket, explaining the requirement for multivalency. Multivalent binding has therefore been explored, and the binding affinity of trivalent and tetravalent carbohydrate constructs to ASGPR is 100-1000 folds stronger compared to monovalent ligands due to the glyco-cluster effect.

Bi— and triantennary GalNAc ligands conjugated to SiRNAs demonstrated significantly higher levels of GalNAc-siRNA in the livers of C57BL/6 mice from subcutaneous administration with 94% of the GalNAc-siRNA localized in hepatocytes. Further, these siRNA conjugates mediated efficient gene silencing. Further studies reported that anti-sense oligonucleotides (ASOs) linked to triantennary GalNAc were up to 10-fold more potent than the parent ASOs in mouse models.

Carbohydrate-protein interactions play an important role in biological processes such as receptor-mediated endocytosis and have been applied to cell recognition studies as well as designs for biomedical materials. Carbohydrate-terminating dendrimers (glycodendrimers) are endowed with enhanced binding affinities with allied receptors, which enables them to interact with specific cell types with avidity and selectivity for targeted drug delivery. Introduction of carbohydrate moieties in the drug delivery platform also provides biocompatibility, as well as increases water solubility of the dendrimer complexes.

Triantennary-GalNAc provides effective multivalent binding to ASGPR on hepatocytes. Therefore, in preferred embodiments, the dendrimers are modified at one or more surface terminal groups (e.g., —OH) with one or more triantennary-GalNAc groups.

Triantennary GalNAc modification of a dendrimer gives rise to a set of three GalNAc at each surface terminal group. In some embodiments, three β-GalNAc molecules are grafted via one or more linkers onto a building block to yield an AB₃ building block (i.e., triantennary GalNAc dendron) suitable for conjugation to the surface functional groups of the dendrimers.

In one embodiment, three β-GalNAc molecules are grafted via one or more linkers onto a propargylated pentaerythritol building block to yield an AB₃ building block suitable for conjugation to the surface functional groups of the dendrimers as shown below.

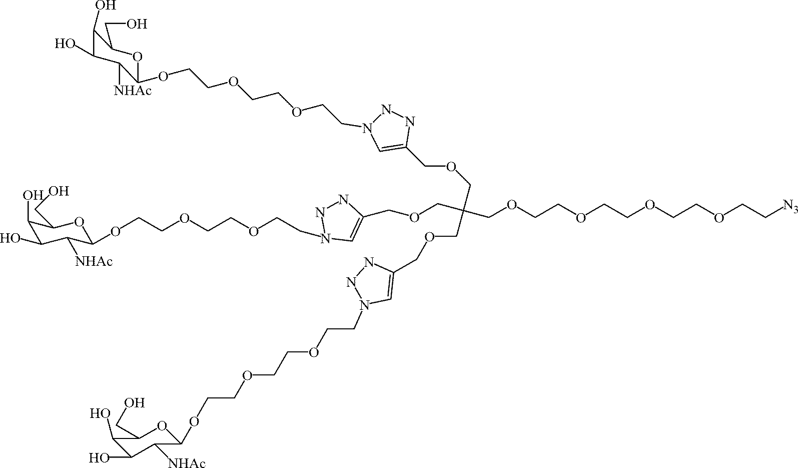 [SMILES: CC(=O)NC1C(OCCOCCOCCN2C=C(COCC(COCCOCCOCCOCCN=[N+]=[N-])(COCC3=CN(CCOCCOCCOC4OC(CO)C(O)C(O)C4NC(C)=O)NN3)COCC3=CN(CCOCCOCCOC4OC(CO)C(O)C(O)C4NC(C)=O)NN3)NN2)OC(CO)C(O)C1O]

In some embodiments, conjugation of triantennary-GalNAc through one or more surface groups occurs via about 1%, 2%, 3%, 4%, 5%,, 6%, 7%, 8%, 9%, 10%, 15%, 20%, 25%, or 30% of the total available surface functional groups, preferably hydroxyl groups, of the dendrimers prior to the conjugation. In other embodiments, the conjugation of triantennary-β-GalNAc occurs on less than 5%, less than 10%, less than 15%, less than 20%, less than 25%, less than 30%, less than 35%, less than 40%, less than 45%, less than 50% of total available surface functional groups of the dendrimers prior to the conjugation. In preferred embodiments, dendrimers are conjugated to an effective amount of triantennary-βGalNAc for binding to ASGPR and/or targeting and on hepatocytes, whilst conjugated to an effective amount of active agents to treat, prevent, and/or image the liver disease or disorder.

### C. Dendrimer Complexes

Dendrimers modified with triantennary GalNAc (dendrimer-triantennary GalNAc) can include one or more therapeutic or prophylactic agents complexed, covalently conjugated, or intra-molecularly dispersed or encapsulated with the dendrimer. Conjugation of one or more agents to the dendrimer component of a dendrimer-triantennary GalNAc complex can occur prior to, at the same time as, or subsequent to conjugation of the dendrimer with the triantennary GalNAc. Compositions and methods for conjugating agents with dendrimers are known in the art, and are described in detail in U.S. Published Application Nos. US 2011/0034422, US 2012/0003155, and US 2013/0136697.

In some embodiments, one or more active agents are covalently attached to the dendrimer component of the dendrimer-triantennary GalNAc. In some embodiments, the active agents are functionalized for conjugation to the dendrimer, optionally via one or more spacers or linking moieties. The functionalized active agents and/or linking moieties are designed to have a desirable release rate of the active agents from the dendrimer-triantennary GalNAc in vivo. The functionalized active agents and/or linking moieties can be designed to be cleaved hydrolytically, enzymatically, or combinations thereof, to provide for the sustained release of the active agents in vivo. In the case where cleavable forms are desired, both the composition of the linking moiety and its point of attachment to the active agent, are selected so that cleavage of the linking moiety releases either an active agent, or a suitable prodrug thereof. In some embodiments, the functionalized active agents and/or linking moieties are designed to be cleaved at a minimal or insignificant rate in vivo. The composition of the linking moiety can also be selected in view of the desired release rate of the active agents. In preferred embodiments, one or more active agents are functionalized to be non-cleavable or minimally cleavable from the dendrimer-triantennary GalNAc in vivo, for example via one or more amide or ether linkages, optionally, with one or more spacers/linkers.

In some embodiments, the attachment of an agent to the dendrimer occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the active agent. In some cases, an ester bond is introduced for releasable form of active agents. In other cases, an amide bond is introduced for non-releasable form of active agents.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety can be chosen in view of the desired release rate of the active agents. In addition, the one or more organic functional groups can be chosen to facilitate the covalent attachment of the active agents to the dendrimers. In preferred embodiments, the attachment can occur via an appropriate spacer that provides a disulfide bridge between the agent and the dendrimer. The dendrimer- triantennary GalNAc complexes are capable of rapid release of the agent in vivo by thiol exchange reactions, under the reduced conditions found in body.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the active agent and the dendrimers.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given active agent can be selected in view of the linking moiety desired, as well as the structure of the agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more active drugs are encapsulated, associated, and/or conjugated to the dendrimer-triantennary GalNAc complex, preferably through one or more surface groups of the dendrimer at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of a dendrimer to an active agent occurs prior to conjugation of the dendrimer with triantennary GalNAc. In some embodiments, conjugation of active agents and/or linkers to the dendrimer occurs through one or more surface and/or interior groups. Thus, in some embodiments, the conjugation of active agents/linkers occurs via about 1%, 2%, 3%, 4%, 5%,, 6%, 7%, 8%, 9%, or 10% of the total available surface functional groups, preferably hydroxyl groups, of the dendrimers prior to the conjugation. In other embodiments, the conjugation of active agents/linkers occurs on less than 5%, less than 10%, less than 15%, less than 20%, less than 25%, less than 30%, less than 35%, less than 40%, less than 45%, less than 50% of total available surface functional groups of the dendrimers prior to the conjugation and/or the modification with triantennary-β-GalNAc. In preferred embodiments, dendrimer complexes retain an effective amount of surface functional groups for modification with triantennary-β-GalNAc for targeting to hepatocytes, whilst conjugated to an effective amount of active agents to treat, prevent, and/or image a disease or disorder.

### D. Coupling Agents and Spacers

Dendrimer complexes can be formed of therapeutically active agents or compounds conjugated or bound to the dendrimers. Optionally, the active agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an agent can be designed to provide a releasable or non-releasable form of the dendrimer-active complexes in vivo. In some embodiments, the attachment occurs via an appropriate spacer that provides an ester bond between the agent and the dendrimer. In some embodiments, the attachment occurs via an appropriate spacer that provides an amide or ether bond between the agent and the dendrimer. In preferred embodiments, one or more spacers/linkers between a dendrimer and an agent are added to achieve desired and effective release kinetics in vivo.

The spacer can be either a single chemical entity or two or more chemical entities linked together to bridge the dendrimer and the active agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), and cyclo(Arg-Ala-Asp-d-Tyr-Cys). In some embodiments, the spacer includes a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. In some embodiments, the spacer includes thiosalicylic acid and its derivatives, (4-succinimidyloxycarbonyl-methyl-alpha-2-pyridylthio)toluene, (3-[2-pyridithio]propionyl hydrazide. In some embodiments, the spacer includes maleimide terminations wherein the spacer includes polymer or small chemical entity such as bis-maleimido diethylene glycol and bis-maleimido triethylene glycol, Bis-Maleimidoethane, bismaleimidohexane. In some embodiments, the spacer includes vinylsulfone such as 1,6-Hexane-bis-vinylsulfone. In some embodiments, the spacer includes thioglycosides such as thioglucose. In other embodiments, the spacer includes reduced proteins such as bovine serum albumin and human serum albumin, any thiol terminated compound capable of forming disulfide bonds. In particular embodiments, the spacer includes polyethylene glycol having maleimide, succinimidyl and thiol terminations.

Agents and/or targeting moiety can be either covalently attached or intra-molecularly dispersed or encapsulated in dendrimer. The dendrimer is preferably a generation 4, generation 5, generation 6, generation 7, generation 8, generation 9, or generation 10 PAMAM dendrimer having hydroxyl terminations. In preferred embodiments, the dendrimer is linked to agents via a spacer ending in disulfide, ester, ether, or amide bonds.

In some embodiments, a non-releasable form of the dendrimer/active agent complex provides enhanced therapeutic efficacy as compared to a releasable form of the same dendrimer/active agent complex. Therefore, in some embodiments, one or more active agent(s) is conjugated to the dendrimer via a spacer that is attached to the dendrimer in a non-releasable manner, for example, by an ether or amide bond. In some embodiments, one or more active agent(s) is attached to the spacer in a non-releasable manner, for example, by an ether or amide bond. Therefore, in some embodiments, one or more active agent(s) is attached to the dendrimer via a spacer that is attached to the dendrimer, and to the active agent(s) in a non-releasable manner. In an exemplary embodiment, one or more active agent(s) is attached to the dendrimer via a spacer that is attached to the dendrimer and the active agent(s) via amide and/or ether bonds. An exemplary spacer is polyethylene glycol (PEG).

### 1. Dendrimer Conjugation to Active Agents via Ether Linkages

In some embodiments, compositions include a hydroxyl-terminated triantennary GalNAc-modified dendrimer conjugated to an active agent via an ether linkage, optionally with one or more linkers/spacers are described.

In preferred embodiments, the covalent bonds between the surface groups of the dendrimers and the linkers, or the dendrimers and the active agent (if conjugated without any linking moieties) are stable under in vivo conditions, i.e., minimally cleavable when administered to a subject and/or excreted intact from the body. For example, in preferred embodiments, less than 10%, 5%, 4%, 3%, 2%, 1%, 0.5%, 0.4%, 0.3%, 0.2%, 0.1%, or less than 0.1% of the total dendrimer complexes have active agent cleaved off without 24 hours, or 48 hours, or 72 hours after in vivo administration. In one embodiments, these covalent bonds are ether bonds. In further preferred embodiments, the covalent bonds between the surface groups of the dendrimers and the linkers, or the dendrimers and the active agent (if conjugated without any linking moieties) are not hydrolytically or enzymatically cleavable bonds such as ester bonds.

In some embodiments, one or more hydroxyl groups of hydroxyl-terminated dendrimers conjugate to one or more linking moieties and one or more active agents via one or more ether bonds as shown in Formula (I) below.

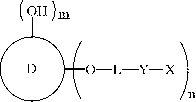

wherein D is a generation 2 to generation 10 poly(amidoamine) (PAMAM) dendrimer; L is one or more linking moieties or spacers; X is an active agent or a derivative, an analogue or a prodrug thereof; n is an integer from 1 to 100; and m is an integer from 16 to 4096;
and Y is a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, and ethers (—O—), wherein R is an alkyl group, an aryl group, or a heterocyclic group. Preferably, Y is a bond or linkage that is minimally cleavable in vivo.

In preferred embodiments, Y is a secondary amide (—CONH—).

In one embodiment, D is a generation 4 or generation 6 hydroxyl-terminated PAMAM dendrimer; L is one or more linking or spacer moieties; X is an angiotensin II receptor blocker, Farnesoid X receptor agonist, death receptor 5 agonist, sodium-glucose cotransporter type-2 inhibitor, lysophosphatidic acid 1 receptor antagonist, endothelin-A receptor antagonist, PPARδ agonist, AT1 receptor antagonist, CCR5/CCR2 antagonist, anti-fibrotic agent, anti-inflammatory agent, and/or anti-oxidant agent, or a derivative, an analogue or a prodrug thereof; n is about 5-15; m is an integer about 49-59; and where n+m=64.

In one embodiment, Y is a secondary amide (—CONH—).

### E. Therapeutic, Prophylactic and Diagnostic Agents

In some embodiments, the triantennary-GalNAc modified dendrimers are complexed with or conjugated to one or more therapeutic, prophylactic and diagnostic agents. Agents to be included in the dendrimer complex to be delivered can be proteins or peptides, sugars or carbohydrate, nucleic acids or oligonucleotides, lipids, small molecules (e.g., molecular weight less than 2500 Daltons, preferably less than 2000 Daltons, more preferably less than 1500 Dalton, more preferably 300-700 Dalton), or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the active agent is a therapeutic antibody.

Dendrimers have the advantage that multiple therapeutic, prophylactic, and/or diagnostic agents can be delivered with the same dendrimers. In some embodiments, one or more types of active agents can be encapsulated, complexed or conjugated to the dendrimer. In particular embodiments, the dendrimers are complexed with or conjugated to two or more different classes of agents, providing simultaneous delivery with different or independent release kinetics at the target site. For example, one dendrimer can be covalently linked to one or more PPARδ agonists and to one or more angiotensin II receptor blockers. In another embodiment, the dendrimers are covalently linked to at least one detectable moiety and at least one class of agents. In a further embodiment, dendrimer complexes each carrying different classes of agents are administered simultaneously for a combination treatment. In one embodiment, the dendrimer composition has multiple agents, such as a chemotherapeutic agent, immunotherapeutic agent, an anti-fibrotic agent, a steroid to decrease swelling, antibiotic, anti-angiogenic agent, and/or a diagnostic agent, complexed with or conjugated to the dendrimers.

The selective targeting of triantennary-GalNAc modified dendrimers allows less active agent to be administered to achieve the same therapeutic effect compared to the same active agent without conjugation to the dendrimers or compared to the same active agent conjugated to dendrimers without modification with triantennary-β-GalNAc, thus, reducing dose-related cytotoxicity and/or other side effects side effects associated with the active agent. Dendrimer can also increase solubility of the one or more therapeutic, prophylactic, and/or diagnostic agents to be delivered. For example, telmisartan is a very hydrophobic drug but the conjugate with dendrimer is highly water-soluble and the water solubility is around 60 mg/ml.

Active agents can also be a pharmaceutically acceptable prodrug of any of the compounds described below. Prodrugs are compounds that, when metabolized in vivo, undergo conversion to compounds having the desired pharmacological activity. Prodrugs can be prepared by replacing appropriate functionalities present in the compounds described above with “pro-moieties” as described, for example, in H. Bundgaar, Design of Prodrugs (1985). Examples of prodrugs include ester, ether or amide derivatives of the compounds described above, polyethylene glycol derivatives of the compounds described above, N-acyl amine derivatives, dihydropyridine pyridine derivatives, amino-containing derivatives conjugated to polypeptides, 2-hydroxybenzamide derivatives, carbamate derivatives, N-oxides derivatives that are biologically reduced to the active amines, and N-mannich base derivatives. For further discussion of prodrugs, see, for example, Rautio, J. et al. Nature Reviews Drug Discovery. 7:255-270 (2008).

Active agents include therapeutic agents that have been shown to have efficacy for treating and preventing one or more liver diseases or disorders. Exemplary therapeutic agents include angiotensin II receptor blockers, Farnesoid X receptor (FXR) agonists, sodium-glucose cotransporter type-2 (SGLT2) inhibitors, apoptosis signal-regulating kinase 1 (ASK-1) inhibitors, pyridinone derivatives, FGF-21 analogs, FGF-19 analogs, lysophosphatidic acid (LPA) 1 receptor antagonists, endothelin-A receptor antagonist, PPARα/δ agonists, AT1 receptor antagonists, CCR5/CCR2 inhibitors, activators of death receptors 5 (DR5), anti-fibrotic agents, anti-inflammatory agents, and/or anti-oxidant agents. In some embodiments, dendrimer-triantennary GalNAc are complexed with or conjugated to one or more angiotensin II receptor blockers, FXR agonists, SGLT2 inhibitors, ASK-1 inhibitors, pyridinone derivatives, FGF-21 analogs, FGF-19 analogs, LPA1 receptor antagonists, endothelin-A receptor antagonist, PPARα/δ agonists, AT1 receptor antagonists, CCR5/CCR2 antagonists, activators of DR5, anti-fibrotic agents, anti-inflammatory agents, anti-oxidant agents, or combinations thereof.

Peroxisome proliferator-activated receptor delta (PPARδ), a member of the nuclear receptor family, is emerging as a key metabolic regulator with pleiotropic actions on various tissues including fat, skeletal muscle, and liver. PPARδ agonist protects hepatocytes from cell death by reducing ROS generation of hepatocytes, leading to less liver fibrosis. Exemplary PPAR-δ agonists have been previously described. In some embodiments, the PPAR-δ agonists are indanylacetic acid derivatives carrying 4-thiazolyl-phenoxy tail groups as described in Rudolph J et al., J. Med. Chem. 2007, 50, 5, 984-1000 (2007). Exemplary PPAR-δ agonists have been previously described, for example, by Ham J et al., Eur J Med Chem. 53:190-202 (2012). Thus, in some embodiments, triantennary-β-GalNAc modified dendrimers are complexed, covalently conjugated, or intra-molecularly dispersed or encapsulated with one or more PPARδ agonists. In one embodiment, triantennary-β-GalNAc modified dendrimers are complexed, covalently conjugated, or intra-molecularly dispersed or encapsulated with one or more PPAR-δ agonists such as GW0742, GW501516, elafibranor, or a derivative or analogue or prodrug thereof.

Sodium-glucose cotransporter type-2 (SGLT2) inhibitors are glucose-lowering agents that improve glucose control while promoting weight loss and lowering serum uric acid levels. Beneficial effects of SGLT2 inhibitors on fatty liver were reported by Scheen AJ. Diabetes Metab. 2019;45(3):213-223; Omori et al., Metab. Clin. Exp. 2019 Jul 11. Exemplary SGLT2 inhibitors include Phlorizin, T-1095, canagliflozin, dapagliflozin, ipragliflozin, tofogliflozin, empagliflozin, luseogliflozin, ertugliflozin, and remogliflozin etabonate. Thus, in some embodiments, triantennary-β-GalNAc modified dendrimers are complexed, covalently conjugated, or intra-molecularly dispersed or encapsulated with one or more SGLT2 inhibitors.

Lysophosphatidic acid (LPA) is a lipid mediator, which is produced mainly by activated platelets via hydrolysis of lysophosphatidylcholine by autotaxin (ATX). LPA is a bioactive lipid implicated in several functions, including proliferation, apoptosis, migration, and cancer cell invasion. LPA and LPA1 receptor (LPA1R) are increased in many inflammatory states, including pulmonary fibrosis, liver fibrosis, and systemic sclerosis. LPA exerts various physiological effects on the receptors of parenchymal cells and LPA1R antagonists showed anti-fibrotic effect on liver fibrosis, lung fibrosis and scleroderma model. Exemplary LPA1 receptor antagonists include BMS-986202, BMS-986020, VPC12249, AM966, AM095, Ki16425, and Ki16198. Thus, in some embodiments, triantennary-β-GalNAc modified dendrimers are complexed, covalently conjugated, or intra-molecularly dispersed or encapsulated with one or more LPA1 receptor antagonists such as BMS-986202, BMS-986020, VPC12249, AM966, AM095, Ki16425, Ki16198, or a derivative or analogue or prodrug thereof.

Anti-fibrotic effects of ambrisentan, an endothelin-A receptor antagonist, has been shown in a non-alcoholic steatohepatitis mouse model (World J Hepatol. 2016 Aug 8; 8(22): 933-941). Exemplary endothelin receptor antagonists include Sitaxentan, Ambrisentan, Macitentan, and Zibotentan. Thus, in some embodiments, triantennary-β-GalNAc modified dendrimers are complexed, covalently conjugated, or intra-molecularly dispersed or encapsulated with one or more endothelin receptor antagonists such as Sitaxentan, Ambrisentan, Macitentan, and Zibotentan, or a derivative or analogue or prodrug thereof.

As oxidative stress has been implicated in the pathogenesis of NAFLD, the role of anti-oxidant agents such as vitamin E, which is known to react with reactive oxygen species (ROS), blocking the propagation of free radical reactions in a wide range of oxidative stress situations. In one embodiment, the active agent is vitamin E or a derivative or analogue or prodrug thereof.

Typically, one or more active agents are functionalized, for example with ether, ester, or amide linkage, optionally, with one or more spacers/linkers, for ease of conjugation with the dendrimers and/or for desired release kinetics. In preferred embodiments, one or more active agents are functionalized to be non-cleavable or minimally cleavable from the dendrimers in vivo, for example via ether or amide optionally, with one or more spacers/linkers.

In preferred embodiments, the one or more active agents delivered via triantennary-GalNAc modified dendrimers are released from the dendrimer complexes after administration to a mammalian subject in an amount effective to be therapeutically effective at the target cells, tissues, regions for at least 1 day, 2 days, 3 days, 4 days, 5 days, 6 days, preferably at least a week, 2 weeks, or 3 weeks, more preferably at least a month, two months, three months, four months, five months, or six months.

### 1. Angiotensin II Receptor Blocker (ARB)

In some embodiments, the triantennary-GalNAc modified dendrimers are complexed with or conjugated to one or more Angiotensin II Receptor Blockers (ARB). The renin angiotensin pathway in hepatic stellate cells induces reactive oxygen species and accelerates hepatic fibrosis. In response to sustained liver injury, the renin angiotensin system (RAS) locally accelerates inflammation, tissue repair and fibrogenesis by production of angiotensin II (Ang II), a vasoconstricting agonist implicated in the pathogenesis of liver fibrosis. RAS is described as a single cascade where renin converts angiotensinogen into angiotensin I (Ang I), which is converted to angiotensin II (Ang II) by angiotensin converting enzyme (ACE). Ang II mediates biological responses through two G-protein-coupled receptors, the Ang II receptor type 1 (AT1) and Ang II receptor type 2 (AT2). However, the fibrogenic actions of Ang II are mostly mediated by angiotensin receptor AT1.

Among the emerging treatment approaches for NAFLD is the anti-hypertensive agent telmisartan, which has positive effects on liver, lipid, and glucose metabolism, especially through its action on the renin-angiotensin system, by blocking the ACE/AngII/AT1 axis and increasing ACE2/Ang(1-7)/Mas axis activation.

Thus, in some embodiments, triantennary-β-GalNAc modified dendrimers are complexed, covalently conjugated, or intra-molecularly dispersed or encapsulated with one or more angiotensin II receptor blockers for treating, alleviating, or preventing one or more liver diseases or disorders such as nonalcoholic fatty liver disease, non-alcoholic steatohepatitis, drug-induced liver failure, hepatitis, liver fibrosis, liver cirrhosis, or combinations thereof.

In some embodiments, angiotensin II receptor blockers are functionalized, for example, with ether, ester, or amide linkage, optionally, with one or more spacers/linkers, for ease of conjugation with the dendrimers and/or for desired release kinetics. In preferred embodiments, angiotensin II receptor blockers are functionalized to be non-cleavable or minimally cleavable from the dendrimers in vivo, for example via ether or amide optionally, with one or more spacers/linkers. In preferred embodiments, angiotensin II receptor blockers or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG).

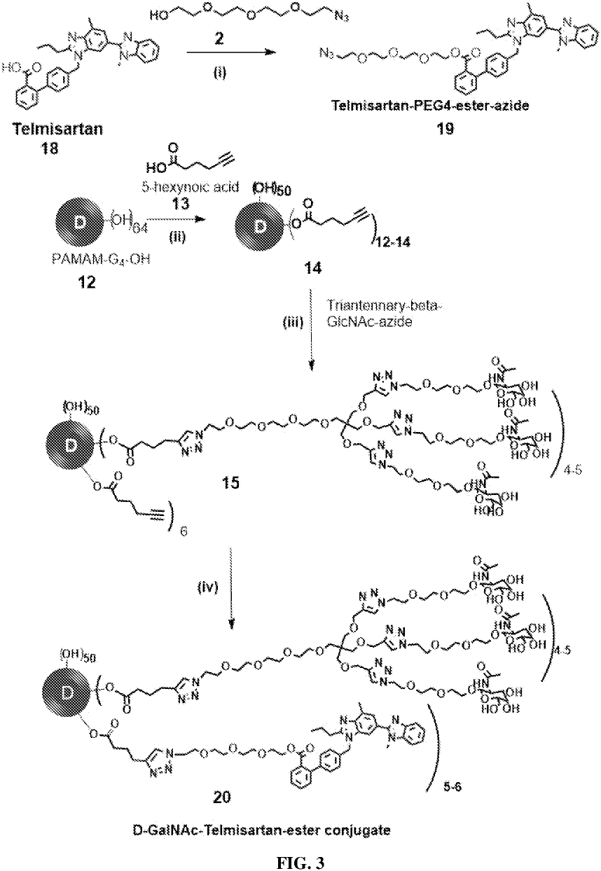

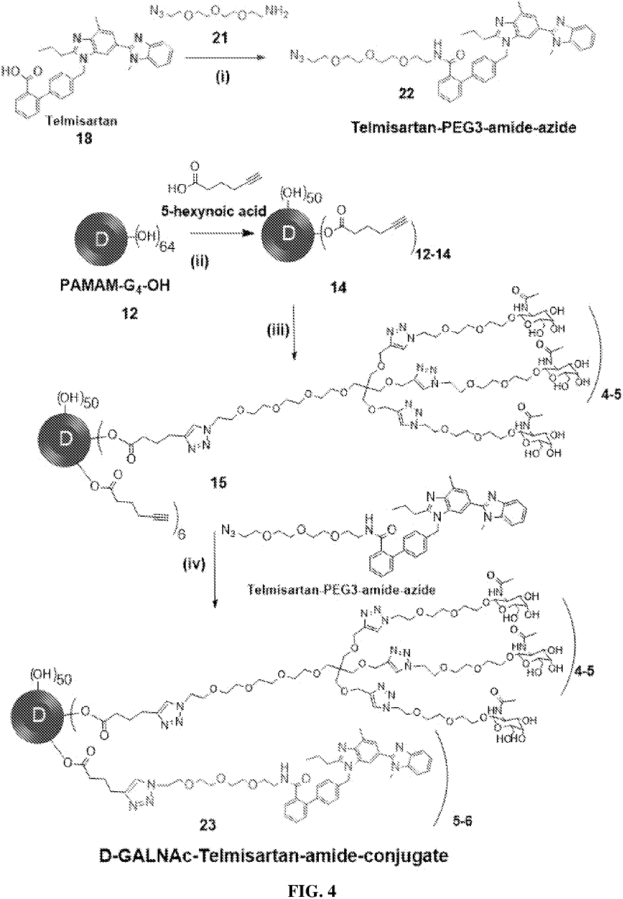

In one embodiment, triantennary-β-GalNAc modified-dendrimers are complexed, covalently conjugated, or intra-molecularly dispersed or encapsulated with telmisartan, or a derivative, analog or prodrug, or a pharmacologically active salt thereof. In some embodiments, telmisartan is functionalized, for example, with ether, ester, or amide linkage, optionally via one or more spacers/linkers such as polyethylene glycol (PEG). Exemplary conjugation of a telmisartan to triantennary-β-GalNAc modified hydroxyl terminated PAMAM dendrimers as dendrimer-telmisartan ester conjugate and dendrimer-telmisartan amide conjugate are shown in FIG. 3 and FIG. 4, respectively.

### 2. Farnesoid X Receptor (FXR) Agonists

In some embodiments, the triantennary-GalNAc modified dendrimers are complexed with or conjugated to one or more agonists of Farnesoid X Receptor (FXR). Farnesoid X receptor (FXR) is a master regulator of bile acid homeostasis through transcriptional regulation of genes involved in bile acid synthesis and cellular membrane transport. Impairment of bile acid efflux due to cholangiopathies results in chronic cholestasis leading to abnormal elevation of intrahepatic and systemic bile acid levels. Obeticholic acid (OCA) is a potent and selective FXR agonist that is 100-fold more potent than the endogenous ligand chenodeoxycholic acid (CDCA).

In some embodiments, triantennary-β-GalNAc modified dendrimers are complexed, covalently conjugated, or intra-molecularly dispersed or encapsulated with one or more FXR agonists such as obeticholic acid and GW4064 for treating, alleviating, or preventing one or more liver diseases or disorders such as nonalcoholic fatty liver disease, non-alcoholic steatohepatitis, drug-induced liver failure, hepatitis, liver fibrosis, liver cirrhosis, or combinations thereof.

In some embodiments, FXR agonists are functionalized, for example with ether, ester, or amide linkage, optionally, with one or more spacers/linkers, for ease of conjugation with the dendrimers and/or for desired release kinetics. In preferred embodiments, FXR agonists are functionalized to be non-cleavable or minimally cleavable from the dendrimers in vivo, for example, via ether or amide optionally, with one or more spacers/linkers.

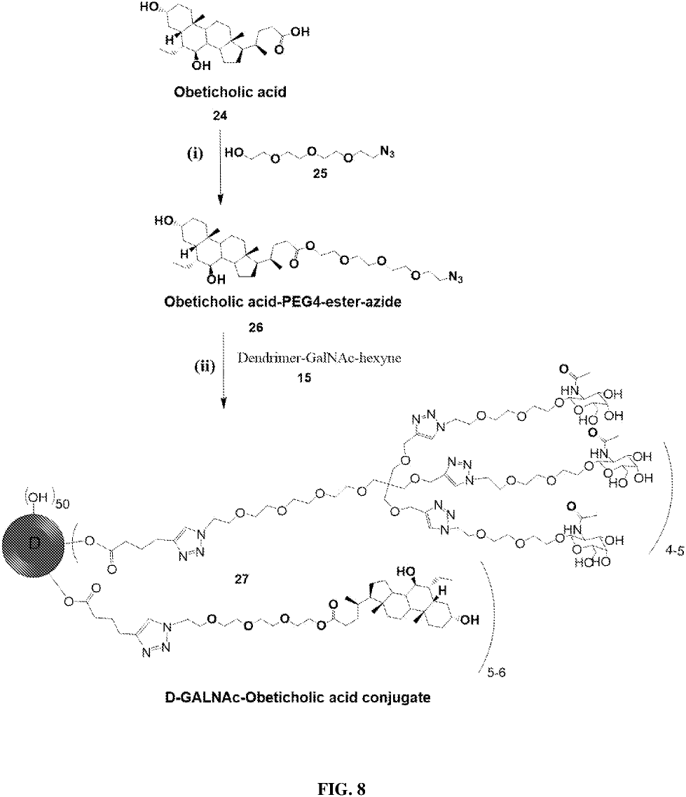

In one embodiment, triantennary-β-GalNAc modified-dendrimers are complexed, covalently conjugated, or intra-molecularly dispersed or encapsulated with obeticholic acid, or a derivative, analog or prodrug, or a pharmacologically active salt thereof. In some embodiments, obeticholic acid is functionalized, for example, with ether, ester, or amide linkage, optionally via one or more spacers/linkers such as polyethylene glycol (PEG). Exemplary conjugation of obeticholic acid to triantennary-β-GalNAc modified hydroxyl terminated PAMAM dendrimers as dendrimer-obeticholic acid ester conjugate are shown in FIG. 8.

### 3. Death Receptor 5 (DR5) Agonists

In some embodiments, the triantennary-GalNAc modified dendrimers are complexed with or conjugated to one or more agonists of Death Receptor 5 (DR5). Death receptor 5 (DR5), also known as TRAIL receptor 2 (TRAIL-R2) and tumor necrosis factor receptor superfamily member 10B (TNFRSF10B), is a cell surface receptor of the TNF-receptor superfamily that binds tumor necrosis factor-related apoptosis-inducing ligand (TRAIL).

In some embodiments, the compositions include one or more DR5 agonists for treating, alleviating, or preventing one or more liver diseases or disorders such as nonalcoholic fatty liver disease, non-alcoholic steatohepatitis, drug-induced liver failure, hepatitis, liver fibrosis, liver cirrhosis, or combinations thereof. A DR5 agonist specifically binds to cells expressing DR5 and triggers an apoptotic cascade resulting in a statistically significant increase in cell death (i.e., apoptosis) as measured in at least one DR5 agonist sensitive cell line (including, but not limited to, the human colon carcinoma cell line Colo 205, or the human lung carcinoma cell line H2122). The DR5 agonist can be an antibody, apo2L/TRAIL, avimer, Fc-peptide fusion protein (such as a peptibody), or a small molecule DR5 agonist. In some embodiments, the DR5 agonist is an avimer (e.g., Nature Biotechnology 23:1556-1561 (2005)), or human TRAIL ligand (e.g., U.S. Pat. Nos. 6,284,236; and 6,998,116) DR5 agonist (e.g., U.S. Publication Nos. 2012/0070432 and 2006/0275838). In some embodiments, the compositions include recombinant soluble TRAIL.

In some embodiments, the compositions include one or more DR5 agonistic antibodies. Exemplary DR5 agonistic antibodies are described by Lee H et al., Biomacromolecules 2016 17 (9), 3085-3093; Yada A et al., Ann. Oncol. 2008, 19, 1060-1067; Ichikawa, K. et al., Nat. Med. 2001, 7, 954-960; Camidge, D. R et al., Clin. Cancer Res. 2010, 16, 1256-1263; Graves, J. D et al., Cancer Cell 2014, 26, 177-189.

In some embodiments, the compositions include one or more DR5 oligomeric peptide and antibody agonists such as those described in Li B et al., J Mol Biol 2006 Aug 18;361(3):522-36, “anti-hDR5 peptides” and “anti-DR5 antibodies” of which are incorporated herein.

In some embodiments, the compositions include one or more disulfide bond-disrupting agents such as those described in Wang M et al., Cell Death Discovery (2019) 5:153; Ferreira, R. B. et al. Oncotarget 8, 28971-28989 (2017); Law, M. E. et al. Breast Cancer Res. 18, 80 (2016); Ferreira, R. B. et al. Oncotarget 6, 10445-10459 (2015). In one embodiments, the compositions include tcyDTDO as shown below.

### Structure I: Chemical Structure of tcyDTDO

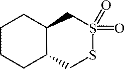 [SMILES: O=S1(=O)C[C@@H]2CCCC[C@H]2CS1]

Some small molecules that directly target DR5 to initiate apoptosis have been previously described (Wang G et al., Nat Chem Biol. 2013 Feb;9(2):84-9). Thus, in some embodiments, the compositions include one or more small molecules that directly target DR5. Some exemplary small molecules that directly target DR5 are shown below.

### Structure II: Chemical Structure of Small Molecules That Directly Target DR5

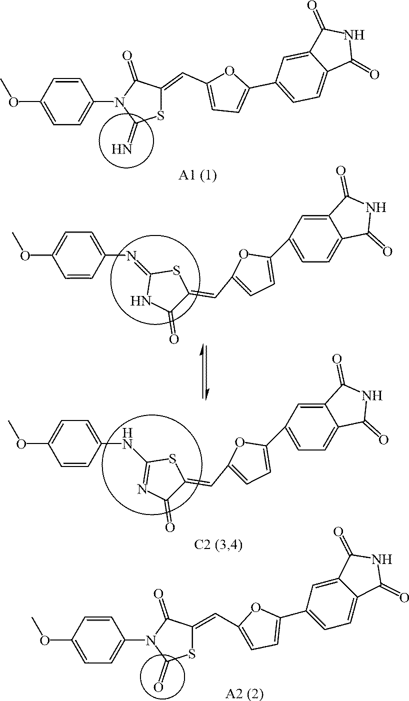

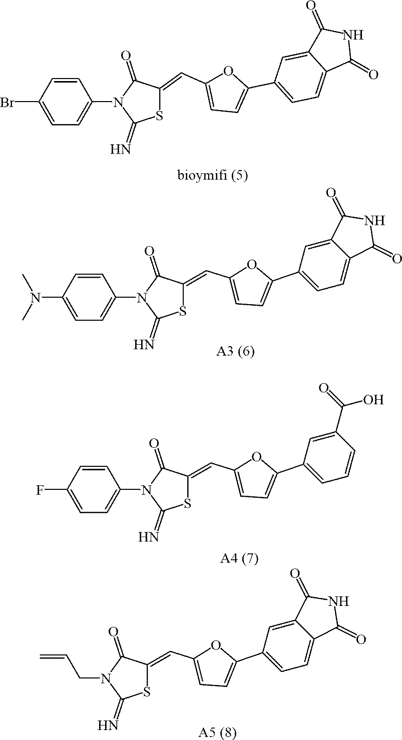

### 4. Anti-Inflammatory Agents

In some embodiments, the triantennary-β-GalNAc modified-dendrimers are complexed, covalently conjugated, or intra-molecularly dispersed or encapsulated with one or more anti-inflammatory agents for treating, alleviating, or preventing one or more liver diseases or disorders such as nonalcoholic fatty liver disease, non-alcoholic steatohepatitis, drug-induced liver failure, hepatitis, liver fibrosis, liver cirrhosis, or combinations thereof. Anti-inflammatory agents reduce inflammation and include steroidal and non-steroidal drugs.

Exemplary anti-inflammatory agents include triamcinolone acetonide, fluocinolone acetonide, dexamethasone, prednisolone, prednisone, methylprednisolone, hydrocortisone acetate, cortisone, diflucortolone, difluprednate, Flucinonide, alclometasone, difluprednate, triamcinolone diacetate, betamethasone, betamethasone valerate, beclometasone and their salts and prodrugs. Glucocortiocoid steroidal antiinflammatories include prednisone, dexamethasone, and corticosteroids such as fluocinolone acetonide and methylprednisolone.

Examples of non-steroidal drugs can be classified into NSAIDS and COX-2 inhibitors. These include ibufenac, acetylsalicylic acid, benoxaprofen, naproxen, alminoproxen, bucloxic acid, ibuprofen, celecoxib, carprofen, etodolac, flufenamic acid, flubiprofen, indomethacin, isoxepac, ketoprofen, mefanemic acid, oxaprozen, oxpinac, parecoxib, phenylbutazone, piroxicam, sulindac, suprofen, tiaprofenic acid, tolmetin, tramadol, valdecoxib salts and their prodrugs.

In preferred embodiments, the active agent is triamcinolone acetonide, prednisone, dexamethasone, or derivatives, analogues or prodrugs, or pharmacologically active salts thereof. Exemplary analogues of triamcinolone acetonide, prednisone, and dexamethasone are shown below (Structure III).

### Structure III A-F: Chemical Structure of Analogues of Triamcinolone Acetonide, Prednisone, Dexamethasone

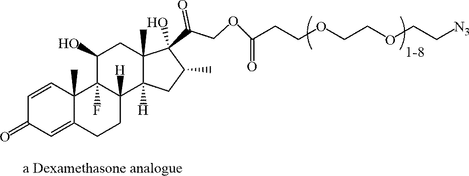

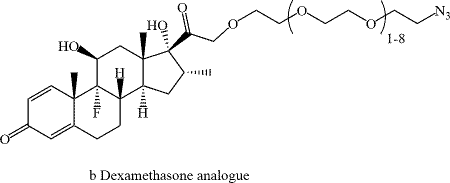

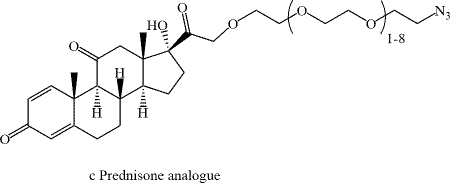

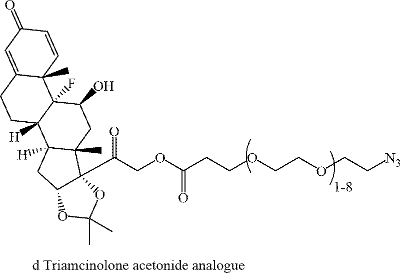

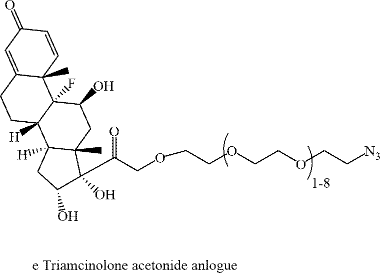

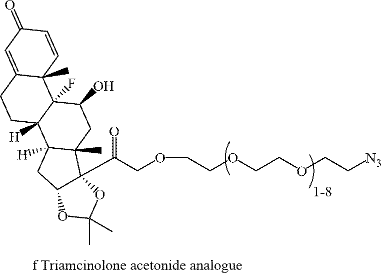

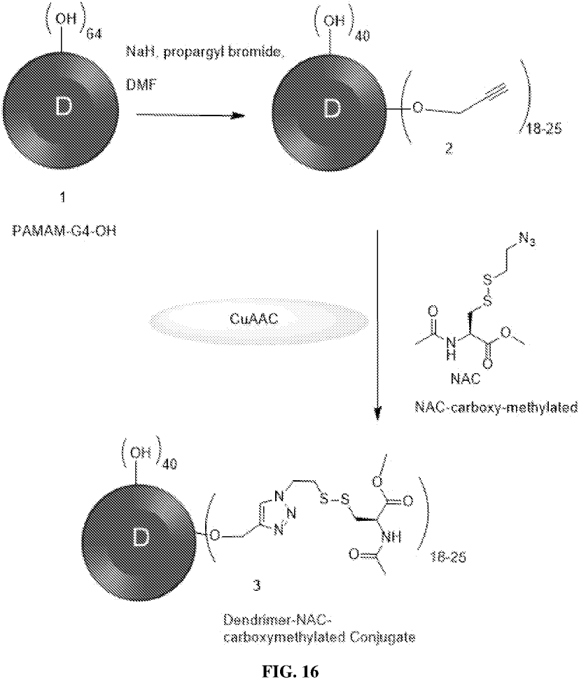

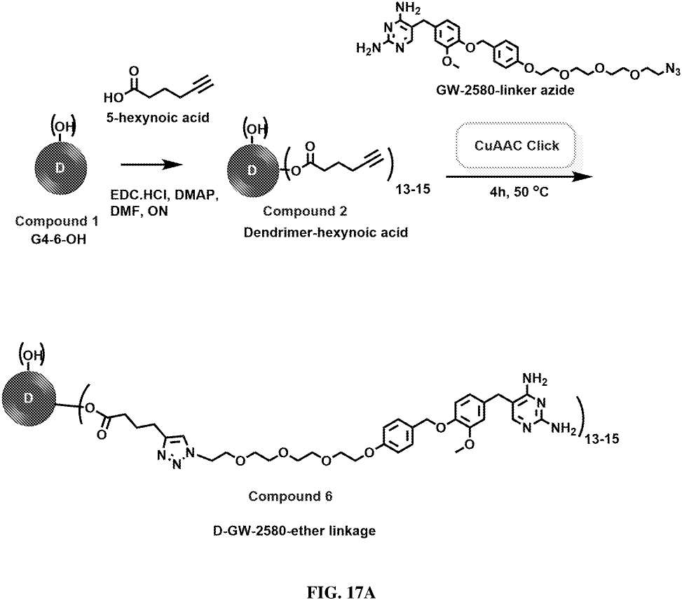

In one embodiment, the anti-inflammatory is N-acetyl-L-cysteine. In a preferred embodiment, N-acetyl-L-cysteine is conjugated to a hydroxyl-terminated PAMAM dendrimer via non-cleavable linkage for minimal release of free N-acetyl-cysteine in vivo after administration. The synthesis route for an exemplary non-releasable (or non-cleavable) form of the dendrimer/N-acetyl-cysteine complexes is shown in FIG. 16. The non-releasable form of the dendrimer/ N-acetyl-cysteine complex provides enhanced therapeutic efficacy as compared to a releasable or cleavable form of the dendrimer/N-acetyl-cysteine complex.

Exemplary immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukin-12, and interleukin-23.

In some embodiments, the anti-inflammatory drug is a synthetic or natural anti-inflammatory protein. Antibodies specific to select immune components can be added to immunosuppressive therapy. In some embodiments, the anti-inflammatory drug is an anti-T cell antibody (e.g., anti-thymocyte globulin or Anti-lymphocyte globulin), anti-IL-2Rα receptor antibody (e.g., basiliximab or daclizumab), or anti-CD20 antibody (e.g., rituximab).

Many inflammatory diseases may be linked to pathologically elevated signaling via the receptor for lipopolysaccharide (LPS), toll-like receptor 4 (TLR4). Thus, in some embodiments, the active agents are one or more TLR4 inhibitors.

### 5. Anti-Fibrotic Agents

In some embodiments, triantennary-β-GalNAc modified dendrimers are complexed, covalently conjugated, or intra-molecularly dispersed or encapsulated with one or more anti-fibrotic therapy agents for treating, alleviating, or preventing one or more liver diseases or disorders such as nonalcoholic fatty liver disease, non-alcoholic steatohepatitis, drug-induced liver failure, hepatitis, liver fibrosis, liver cirrhosis, or combinations thereof. In some embodiments, one or more existing anti-fibrotic therapy agents that have shown to be effective in treating liver fibrosis are complexed with or conjugated to triantennary-β-GalNAc modified dendrimers for enhanced delivery and accumulation inside the hepatocytes. For example, exemplary anti-fibrotic therapy agents include those discussed in Cohen-Naftaly M et al., Therap Adv Gastroenterol. 4(6): 391-417 (2011) and Chang Y et al., J Clin Transl Hepatol. 28; 8(2): 222-229 (2020). In some embodiments, the anti-fibrotic therapy agents are anti-oxidants, anti-TNFα, PPARγ agonists, IFNα agonists, Angiotensin receptor blockers, endothelin receptor antagonists, anticoagulants, FXR agonists, antibodies against connective tissue growth factor, insulin, pegylated interferon, or combinations thereof. In some embodiments, the anti-fibrotic therapy agents are pentoxyphilline, tocopherol, peginterferon 2a, etanercept, recombinant IL-10, pioglitazone, vitamin E, Lovaza (fish oil), polyenylphosphatidylcholine, obeticholic acid, Infliximab, pegylated IFNα-2b, ribavirin, Peg-IFNα-2b, glycyrrhizin, Candesartan, Losartan, Irbesartan, Ambrisentan, FG-3019 (human monoclonal antibody against connective tissue growth factor), Warfarin, insulin, Colchicine, or combinations thereof.

### 6. Immunomodulatory Agents for Treating Liver Cancer

In some embodiments, triantennary-β-GalNAc modified dendrimers are complexed, covalently conjugated, or intra-molecularly dispersed or encapsulated with one or more immunomodulatory agents. The term “immunomodulatory agent” and “immunotherapeutic agent” mean an active agent that elicits a specific effect upon the immune system of the recipient. Immunomodulation can include suppression, reduction, enhancement, prolonging or stimulation of one or more physiological processes of the innate or adaptive immune response to antigen, as compared to a control. Typically, immunomodulatory agents can modulate immune microenvironment for a desired immunological response (e.g., increasing anti-tumor activity, or increasing anti-inflammatory activities sites in need thereof in autoimmune diseases) by targeting one or more immune cells or cell types at a target site, and thus, are not necessarily specific to any cancer type. In some embodiments, the immunomodulatory agents are specifically delivered to kill, inhibit, or reduce activity or quantity of suppressive immune cells such as tumor-associated macrophages for an enhanced anti-tumor response at a tumor site.

Some exemplary immunomodulatory agents used with triantennary-β-GalNAc modified dendrimers include stimulator of interferon genes (STING) agonists, Colony-Stimulating Factor 1 Receptor (CSF1R) inhibitors, Poly(ADP-ribose) polymerase (PARP) inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, phosphatidylinositol-3-kinase (PI3K) inhibitors, Toll-like Receptor 4 (TLR4) agonists, TLR7 agonists, and SHP2 (Src homology-2 domain-containing protein tyrosine phosphatase-2) inhibitors. In preferred embodiments, triantennary-β-GalNAc modified dendrimers are complexed, covalently conjugated, or intra-molecularly dispersed or encapsulated with one or more of STING agonists, CSF1R inhibitors, PARP inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, or combinations thereof.

These dendrimer complexes are particularly suited for targeting one or more suppressive immune cells in the tumor region of the liver as well as reducing the number of cancer cells; reducing the tumor size; inhibiting cancer cell infiltration into peripheral organs; inhibiting tumor metastasis; inhibiting tumor growth; and/or relieving one or more of the symptoms associated with the tumor/cancer. In some embodiments, dendrimers associated with or conjugated to one or more immunomodulatory agents are used in combination with anti-tumor vaccines and/or adoptive cell therapy (ACT) as an adjuvant, for example to increase expression of innate immune genes, infiltration and expansion of activated effector T cells, antigen spreading, and durable immune responses.

In some embodiments, the immunomodulatory agents are any inhibitors targeting one or more of EGFR, ERBB2, VEGFRs, Kit, PDGFRs, ABL, SRC, mTOR, and combinations thereof. In some embodiments, the immunomodulatory agents are one or more inhibitors and analogues thereof, such as crizotinib, ceritinib, alectinib, brigatinib, bosutinib, dasatinib, imatinib, nilotinib, ponatinib, vemurafenib, dabrafenib, ibrutinib, palbociclib, ribociclib, cabozantinib, gefitinib, erlotinib, lapatinib, vandetanib, afatinib, osimertinib, ruxolitinib, tofacitinib, trametinib, axitinib, lenvatinib, nintedanib, pazopanib, regorafenib, sorafenib, sunitinib, vandetanib, bosutinib, dasatinib, dacomitinib, ponatinib, and combinations thereof. In some embodiments, the immunomodulatory agents are tyrosine kinase inhibitors such as HER2 inhibitors, EGFR tyrosine kinase inhibitors. Exemplary EGFR tyrosine kinase inhibitors include gefitinib, erlotinib, afatinib, dacomitinib, and osimertinib.

Additional immunomodulatory agents can include one or more cytotoxic agents that are toxic to one or more immune cells, thus can kill or inhibit one or more types of suppressive immune cells. When delivered selectively to target immune cells such as being conjugated to dendrimers, these agents are able to selectively kill suppressive immune cells and thus alter immunological microenvironment in and around tumors. Cytotoxic immunomodulatory agents include Auristatin E and Mertansine.

### STING Agonists

In some embodiments, the triantennary-GalNAc modified dendrimers are complexed with or conjugated to one or more agonists of Stimulator of interferon genes (STING). Stimulator of interferon genes (STING) is a cytosolic receptor that senses both exogenous and endogenous cytosolic cyclic dinucleotides (CDNs), activating TBK1/IRF3 (interferon regulatory factor 3), NF-κB (nuclear factor κB), and STAT6 (signal transducer and activator of transcription 6) signaling pathways to induce robust type I interferon and proinflammatory cytokine responses. STING is required for the induction of antitumor CD8 T responses in mouse models of cancer. In the tumor microenvironment, T cells, endothelial cells, and fibroblasts, stimulated with STING agonists ex vivo produce type-I IFNs (Corrales, et al., Cell Rep (2015) 11(7):1018-30). By contrast, most studies indicated that tumor cells can inhibit STING pathway activation, potentially leading to immune evasion during carcinogenesis (He, et al., Cancer Lett (2017) 402:203-12; Xia, et al., Cancer Res (2016) 76(22):6747-59). Thus, in some embodiments, the dendrimers are associated with or conjugated to one or more STING agonists or analogues thereof. Exemplary STING agonists include cyclic dinucleotides such as 2′3′ cyclic guanosine monophosphate-adenosine monophosphate (cGAMP) and DMXAA (also known as Vadimezan or ASA404). In one embodiment, triantennary-β-GalNAc modified dendrimers are associated with or conjugated to DMXAA or a derivative, analogue or prodrug thereof. In preferred embodiments, the complex or conjugate of triantennary-β-GalNAc and DMXAA is effective to induce one or more of TNF-α, IP-10, IL-6, IFN-β, and RANTES at the target site.

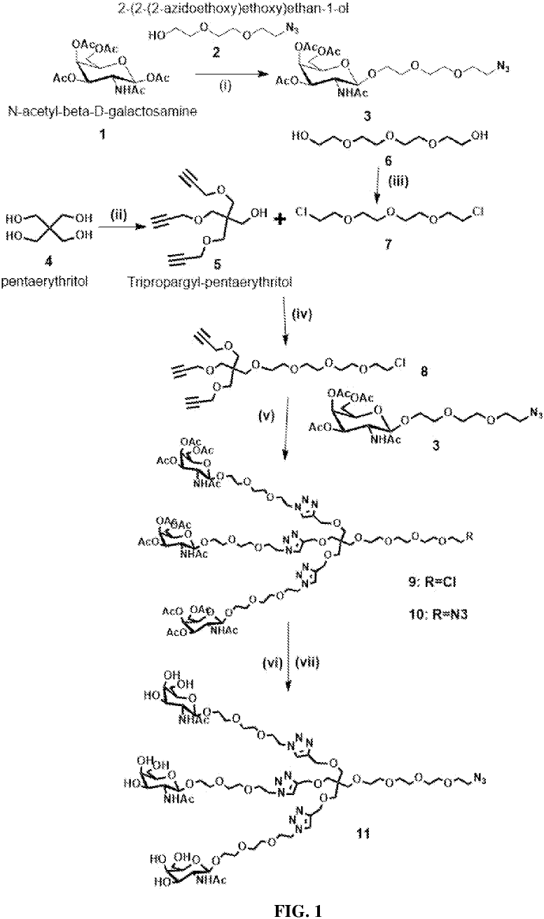

In some embodiments, STING agonists are functionalized, for example with ether, ester, or amide linkage, optionally with one or more spacers/linkers, for ease of conjugation with the dendrimers and/or for desired release kinetics. For example, DMXAA can be modified to DMXAA analogues such as DMXAA ester, DMXAA ether, or DMXAA amide. In preferred embodiments, the STING agonists or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). Exemplary conjugation of a STING agonist, e.g., DMXAA to a dendrimer such as a generation 4 or generation 6 PAMAM dendrimer, is shown in FIG. 1.

In preferred cases, the dendrimer complexes including one or more STING agonists are administered in an amount effective to induce/enhance IFN-β production by tumor-infiltrating APCs (e.g., CD11c+CD11b- or CD11c+CD11b+ cells), induce/enhance one or more of TNF-α, IP-10, IL-6, IFN-β and RANTES, inhibit tumor growth, reduce tumor size, increase rates of long-term survival, improve response to immune checkpoint blockade, and/or induce immunological memory that protects against tumor re-challenge.

### Colony-Stimulating Factor 1 Receptor (CSF1R) Inhibitors

In some embodiments, the triantennary-GalNAc modified dendrimers are complexed with or conjugated to one or more inhibitors of Colony-Stimulating Factor 1 Receptor (CSF1R). CSF1R belongs to the type III protein tyrosine kinase receptor family, and binding of CSF1 or the more recently identified ligand, IL-34, induces homodimerization of the receptor and subsequent activation of receptor signaling (Achkova D, Maher J. Biochem Soc Trans. (2016) 44:333-41). CSF1 receptor (CSF1R)-mediated signaling is crucial for the differentiation and survival of the mononuclear phagocyte system and macrophages in particular (Stanley ER, Chitu V. Cold Spring Harb Perspect Biol (2014), 6(6)). As the intratumoral presence of CSF1R+ macrophages correlates with poor survival in various tumor types (Pedersen MB, et al., Histopathology. (2014), 65:490-500; Zhang QW et al., PLoS One. (2012), 7:e50946), targeting CSF1R signaling in tumor-promoting TAM represents an attractive strategy to eliminate or repolarize these cells. In addition to TAM, CSF1R expression can be detected on other myeloid cells within the tumor microenvironment such as dendritic cells, neutrophils, and myeloid-derived suppressor cells (MDSCs).

A variety of small molecules and monoclonal antibodies (mAbs) directed at CSF1R or its ligand CSF1 are in clinical development both as monotherapy and in combination with standard treatment modalities such as chemotherapy as well as other cancer-immunotherapy approaches. Among the class of small molecules, pexidartinib (PLX3397), an oral tyrosine kinase inhibitor of CSF1R, cKIT, mutant fms-like tyrosine kinase 3 (FLT3), and platelet-derived growth factor receptor (PDGFR)-β, is the subject of the broadest clinical development program in monotherapy, with completed or ongoing studies in c-kit-mutated melanoma, prostate cancer, glioblastoma (GBM), classical Hodgkin lymphoma (cHL), neurofibroma, sarcoma, and leukemia. Additional CSF1R-targeting small molecules, including ARRY-382, PLX7486, BLZ945, and JNJ-40346527, are currently being investigated in solid tumors and cHL. mAbs in clinical development include emactuzumab (RG7155), AMG820, IMC-CS4 (LY3022855), cabiralizumab, MCS110, and PD-0360324, with the latter two being the compounds targeting the ligand CSF1. The phrase “CSF1R inhibitor” is used as a general term for both receptor- and ligand-targeting compounds.

Thus, in some embodiments, the triantennary-β-GalNAc modified dendrimers are associated with or conjugated to one or more agents for reducing or inhibiting the activities of the CSF1R signaling pathway, such as one or more CSF1R inhibitors or one or more compounds targeting the ligand CSF1. In some embodiments the dendrimers are associated with or conjugated to one or more small molecule CSF1R inhibitors or analogues thereof. Exemplary small molecule CSF1R inhibitors are provided in Current Medicinal Chemistry, 2019, 26, 1-23. Exemplary CSF1R-targeting small molecules include pexidartinib (PLX3397, PLX108-01), ARRY-382, PLX7486, BLZ945, JNJ-40346527, and GW 2580. The small molecule CSF1R inhibitors can be functionalized, for example with ether, ester, or amide linkage, optionally with one or more spacers/linkers, for ease of conjugation with the dendrimers and/or for desired release kinetics. In preferred embodiments, the small molecule CSF1R inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG).

The chemical structures of exemplary CSF1R-targeting small molecules or analogs thereof suitable for conjugation to dendrimers are shown below:

### Structure IV: Chemical Structure of CSF1R Inhibitor 1

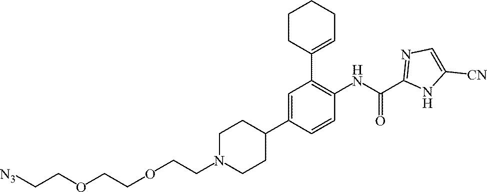 [SMILES: N#Cc1cnc(C(=O)Nc2ccc(C3CCN(CCOCCOCCN=[N+]=[N-])CC3)cc2C2=CCCCC2)[nH]1]

### Structure V: Chemical Structure of CSF1R Inhibitor 2

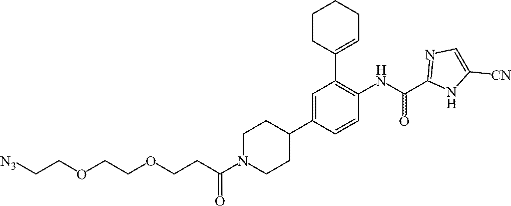 [SMILES: N#Cc1cnc(C(=O)Nc2ccc(C3CCN(C(=O)CCOCCOCCN=[N+]=[N-])CC3)cc2C2=CCCCC2)[nH]1]

### Structure VI: Chemical Structure of CSF1R Inhibitor 3

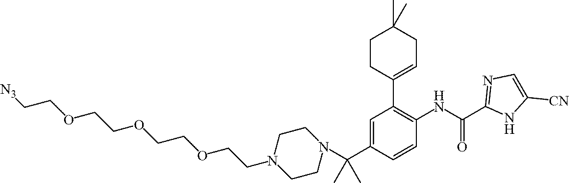 [SMILES: CC1(C)CC=C(c2cc(C(C)(C)N3CCN(CCOCCOCCOCCN=[N+]=[N-])CC3)ccc2NC(=O)c2ncc(C#N)[nH]2)CC1]

### Structure VII: Chemical Structure of CSF1R Inhibitor 4

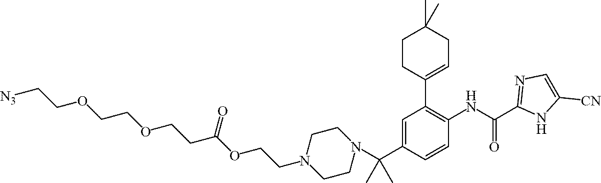 [SMILES: CC1(C)CC=C(c2cc(C(C)(C)N3CCN(CCOC(=O)CCOCCOCCN=[N+]=[N-])CC3)ccc2NC(=O)c2ncc(C#N)[nH]2)CC1]

### Structure VIII: Chemical Structure of CSF1R Inhibitor 5

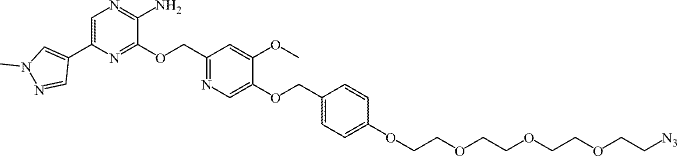 [SMILES: COc1cc(COc2nc(-c3cnn(C)c3)cnc2N)ncc1OCc1ccc(OCCOCCOCCOCCN=[N+]=[N-])cc1]

### Structure IX: Chemical Structure of CSF1R Inhibitor 6

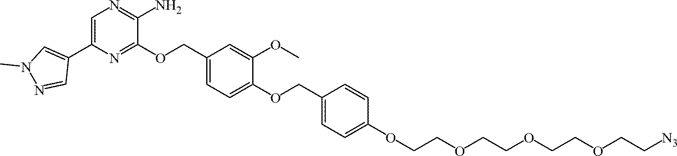 [SMILES: COc1cc(COc2nc(-c3cnn(C)c3)cnc2N)ccc1OCc1ccc(OCCOCCOCCOCCN=[N+]=[N-])cc1]

### Structure X A-B: Chemical Structure of A) a CSF1R-E Analog and B) a Dendrimer-Conjugated CSF1R-E

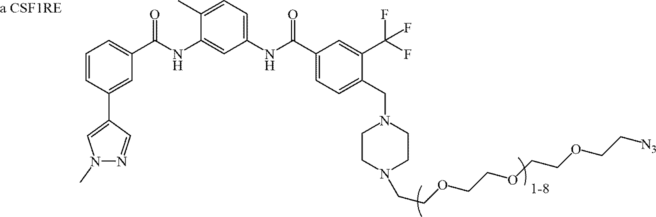

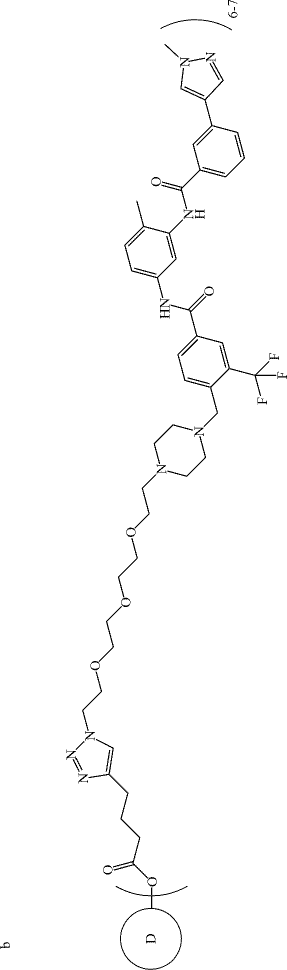

### Structure XI: Chemical Structure of CSF1R-E Analogue 1

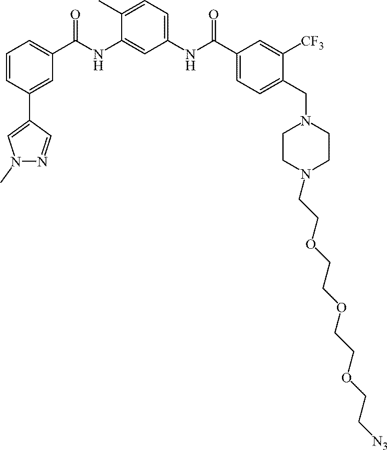 [SMILES: Cc1ccc(NC(=O)c2ccc(CN3CCN(CCOCCOCCOCCN=[N+]=[N-])CC3)c(C(F)(F)F)c2)cc1NC(=O)c1cccc(-c2cnn(C)c2)c1]

The binding affinity of CSF1R-E analogue 1 (Structure XI) is about 13 nm and the binding affinity of dendrimer conjugated CSF1R-E Analogue 1 (for example, via alkyne-azide click chemistry) is about 200 nm. Thus, in preferred embodiments, the CSF1R inhibitors are conjugated to dendrimers with or without a spacer in such a way that it minimizes the reduction in binding affinity towards CSF1R, for example, less than 1-fold, 2-fold, 3-fold, 4-fold, 5-fold, 10-fold, 20-fold, 30-fold, 40-fold, 50-fold, or 100-fold.

### Structure XII: Chemical Structure of CSF1R Inhibitor F

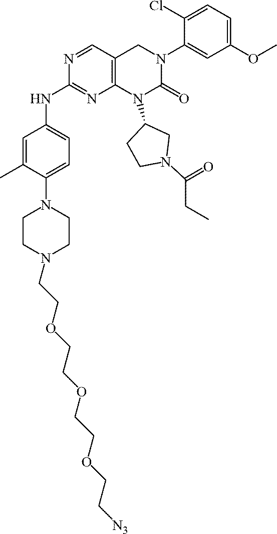

Exemplary CSF1R-targeting mAbs include emactuzumab (RG7155), AMG820, IMC-CS4 (LY3022855), and cabiralizumab. Exemplary mAbs target the ligand CSF1MCS110 and PD-0360324.

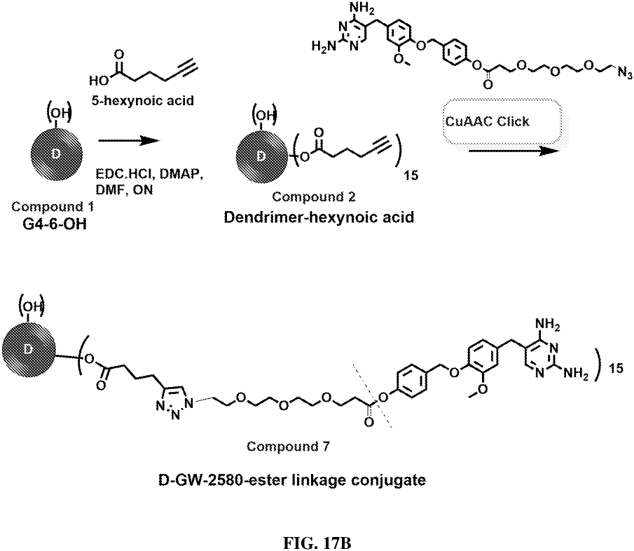

In preferred embodiments, the dendrimers are conjugated to one or more tyrosine kinase inhibitors of CSF1R such as GW2580 (shown as Structure X). The CSF1R inhibitors can be functionalized, for example with ether, ester, or amide linkage, optionally with one or more spacers/linkers, for ease of conjugation with the dendrimers and/or for desired release kinetics. For example, GW2580 can be modified to GW2580 analogues including GW2580 ether, GW2580 ester, and GW2580 amide. In preferred embodiments, the GW2580 or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). Exemplary strategies for conjugating a CSF1R inhibitor, e.g., GW2580, to a dendrimer is shown in FIGS. 17A and 17B.

### Structure XIII: Chemical Structure of GW2580

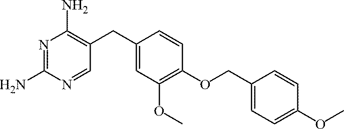 [SMILES: COc1ccc(COc2ccc(Cc3cnc(N)nc3N)cc2OC)cc1]

In one embodiment, the dendrimers are conjugated to a CSF1R inhibitor or an analogue thereof having the following structure.

### Structure XIV: Chemical Structure of AR004

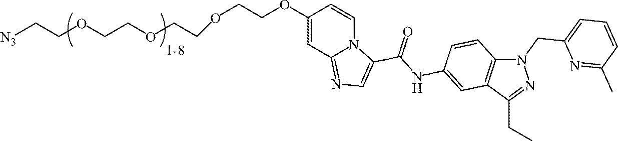

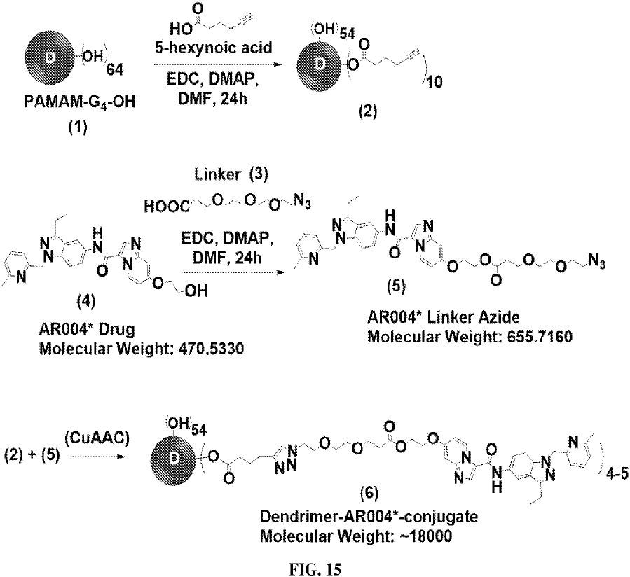

A synthesis route of dendrimers conjugated to AR004 is shown in FIG. 15.

### Poly(ADP-Ribose) Polymerase (PARP) Inhibitors

In some embodiments, the triantennary-GalNAc modified dendrimers are complexed with or conjugated to one or more inhibitors of Poly(ADP-ribose) polymerase (PARP). Poly(ADP-ribose) polymerases (PARPs) are a family of 17 nucleoproteins characterized by a common catalytic site that transfers an ADP-ribose group on a specific acceptor protein using NAD+ as cofactor. Poly(ADP-ribose) polymerase (PARP) inhibitors

Olaparib (C₂₄H₂₃FN₄O₃) was the first PARP inhibitor introduced in clinical practice. Niraparib is a potent and selective inhibitor of PARP-1 and PARP-2. Rucaparib is a potent PARP inhibitor, approved by FDA in December 2016 and by EMA in May 2018 for the treatment, as single agent, of HGSOC patients with gBRCAm or sBRCAm, relapsed after at least two chemotherapy lines.

In some embodiments, dendrimer complexes include one or more PARP inhibitors such as olaparib, niraparib, and rucaparib. The PARP inhibitors can be functionalized, for example with ether, ester, or amide linkage, optionally with one or more spacers/linkers, for ease of conjugation with the dendrimers and/or for desired release kinetics. In preferred embodiments, the PARP inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG).

### VEGFR Tyrosine Kinase Inhibitor

In some embodiments, the triantennary-GalNAc modified dendrimers are complexed with or conjugated to one or more inhibitors of VEGFR Tyrosine Kinase. Tyrosine kinases are important cellular signaling proteins that have a variety of biological activities including cell proliferation and migration. Multiple kinases are involved in angiogenesis, including receptor tyrosine kinases such as the vascular endothelial growth factor receptor (VEGFR). Antiangiogenic tyrosine kinase inhibitors in clinical development primarily target VEGFR-1, -2, -3, epidermal growth factor receptor (EGFR), platelet-derived growth factor receptor (PDGFR), PDGFR-β, KIT, fms-related tyrosine kinase 3 (FLT3), colony stimulating factor-1 receptor (CSF-1R), Raf, and RET.

The VEGFR family includes three related receptor tyrosine kinases, known as VEGFR-1, -2, and -3, which mediate the angiogenic effect of VEGF ligands (Hicklin DJ, Ellis LM. J Clin Oncol. (2005), 23(5):1011-27). The VEGF family encoded in the mammalian genome includes five members: VEGF-A, VEGF-B, VEGF-C, VEGF-D, and placental growth factor (P1GF). VEGFs are important stimulators of proliferation and migration of endothelial cells. VEGF-A (commonly referred to as VEGF) is the major mediator of tumor angiogenesis and signals through VEGFR-2, the major VEGF signaling receptor (Kerbel RS, N Engl J Med. (2008), 358(19):2039-49).

Most notable angiogenesis inhibitors target the vascular endothelial growth factor signaling pathway, such as the monoclonal antibody bevacizumab (Avastin, Genentech/Roche) and two kinase inhibitors sunitinib (SU11248, Sutent, Pfizer) and sorafenib (BAY43-9006, Nexavar, Bayer). Bevacizumab was the first angiogenesis inhibitor that was clinically approved, initially for treatment of colorectal cancer and recently also for breast cancer and lung cancer. The small-molecule tyrosine kinase inhibitors sunitinib and sorafenib target the VEGF receptor (VEGFR), primarily VEGFR-2, and have shown clinical efficacy in diverse cancer types. Both drugs have shown benefit in patients with renal cell cancer (Motzer RJ, Bukowski RM, J Clin Oncol. (2006); 24(35):5601-8). In addition, sunitinib has been approved for treatment of gastrointestinal stromal tumors (GISTs). Sorafenib inhibits Raf serine kinase as well and has been approved for treatment of hepatocellular cancer as well. Cediranib is an oral tyrosine kinase inhibitor of VEGF receptor (VEGFR).

In some embodiments, dendrimers are conjugated to one or more VEGF receptor inhibitors including Sunitinib (SU11248; SUTENT®), Sorafenib (BAY439006; NEXAVAR®), Pazopanib (GW786034; VOTRIENT®), Vandetanib (ZD6474; ZACTIMA®), Axitinib (AG013736), Cediranib (AZD2171; RECENTIN®), Vatalanib (PTK787; ZK222584), Dasatinib, Nintedanib, and Motesanib (AMG706), or analogues thereof.

In some embodiments, the VEGF receptor inhibitors can be functionalized with one or more spacers/linkers, for example with ether, ester, or amide linkage, optionally with one or more spacers/linkers, for ease of conjugation with the dendrimers and/or for desired release kinetics. In preferred embodiments, the one or more VEGF receptor inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). For example, sunitinib can be modified to sunitinib with an ester linkage, or with an amide linkage (FIGS. 3A and 3B). Exemplary conjugation of a VEGF receptor inhibitor, e.g., sunitinib to a dendrimer is shown in FIG. 3A (via a hydroxymethyl linkage) and 3B (via an amide linkage). In one embodiment, the sunitinib analog is N, N-didesethyl sunitinib.

Exemplary VEGF receptor inhibitor analogues with a functional spacer/linkage are shown below in Structure XV, Structure XVI and Structure XVII.

### Structure XV A-B: Chemical Structures of Sorafenib Analogues

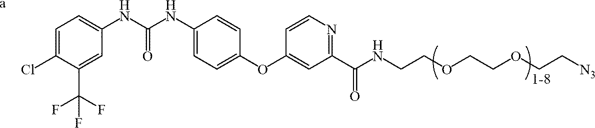

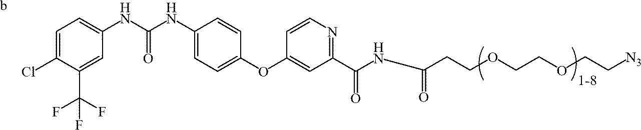

### Structure XVI A-D: Chemical Structures of Nintedanib and Analogues

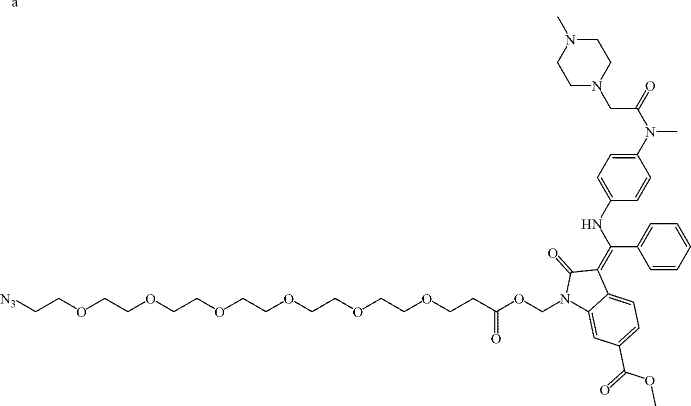

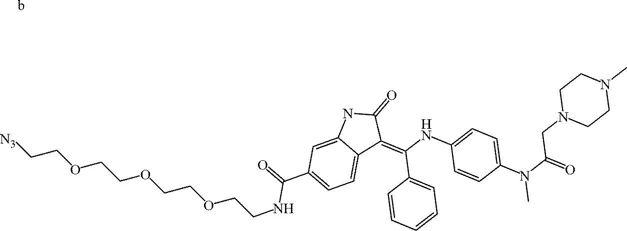

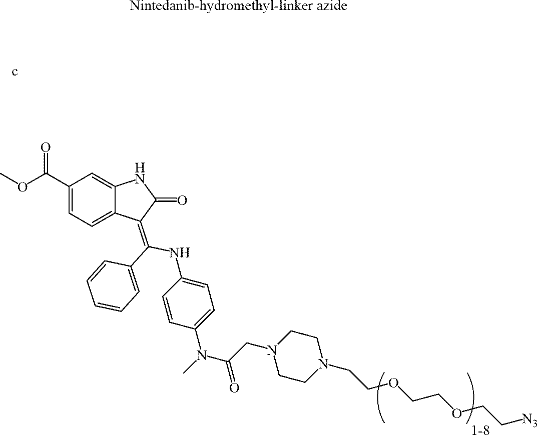

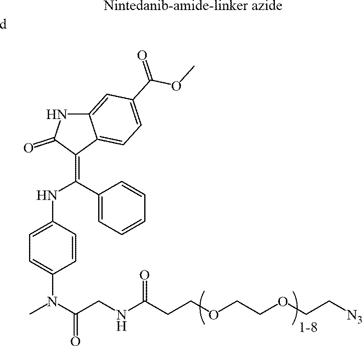

### Structure XVII: Chemical Structures of Orantinib Analogues

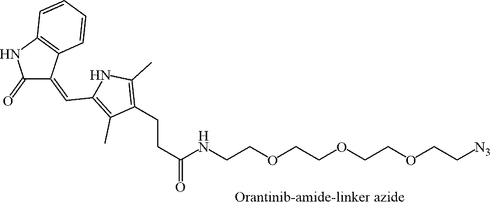

### MEK Inhibitors

In some embodiments, the triantennary-GalNAc modified dendrimers are complexed with or conjugated to one or more inhibitors of MEK. The mitogen-activated protein kinase (MAPK) cascade is a critical pathway for human cancer cell survival, dissemination, and resistance to drug therapy. The MAPK/ERK (extracellular signal regulated kinases) pathway is a convergent signaling node receiving input from numerous stimuli, including internal metabolic stress and DNA damage pathways, and altered protein concentrations, as well as via signaling from external growth factors, cell-matrix interactions, and communication from other cells.

In some embodiments, dendrimers are conjugated to one or more MEK inhibitors. Exemplary MEK inhibitors include Refametinib, Pimasertib, Trametinib (GSK1120212), Cobimetinib (or XL518), Binimetinib (MEK162), Selumetinib, CI-1040 (PD-184352), PD325901, PD035901, PD032901, and TAK-733, or analogues thereof. In preferred embodiments, the MEK inhibitors are functionalized, for example with ether, ester, or amide linkage, optionally with one or more spacers/linkers, for ease of conjugation with the dendrimers and/or for desired release kinetics. In preferred embodiments, the MEK inhibitors or derivatives, analogs or prodrugs thereof are conjugated to the dendrimers via Cu (I) catalyzed alkyne-azide click or thiol-ene click chemistry, optionally via one or more spacers/linkers such as polyethylene glycol (PEG). For example, binimetinib can be modified to binimetinib ester, binimetinib ether, or binimetinib amide; trametinib can be modified to trametinib ether, trametinib ester, or trametinib amide; pimasertib can be modified to pimasertib ester and pimasertib ether etc. Exemplary MEK inhibitors and their analogus thereof are shown below: binimetinib functionalized with a PEG linker and an azide group via an ester linkage (Structure XVIII) and via an ether linkage (Structure XIX); trametinib analogue functionalized with a PEG linker and an azide group via an amide linkage (Structure XX); and pimasertib analogue functionalized with a PEG linker and an azide group via an ester linkage (Structure XXI).

### Structure XVIII: Chemical Structure of Binimetinib Analogue 1

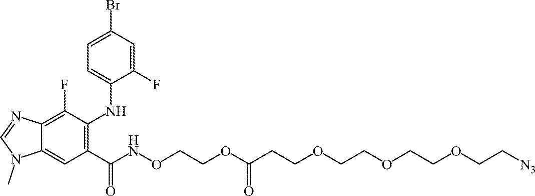 [SMILES: Cn1cnc2c(F)c(Nc3ccc(Br)cc3F)c(C(=O)NOCCOC(=O)CCOCCOCCOCCN=[N+]=[N-])cc21]

### Structure XIX: Chemical Structure of Binimetinib Analogue 2

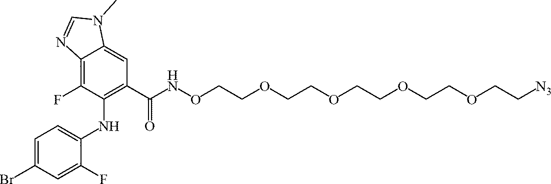 [SMILES: Cn1cnc2c(F)c(Nc3ccc(Br)cc3F)c(C(=O)NOCCOCCOCCOCCOCCN=[N+]=[N-])cc21]

### Structure XX: Chemical Structure of Trametinib Analogue

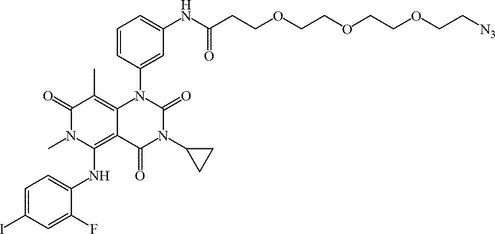 [SMILES: Cc1c(=O)n(C)c(Nc2ccc(I)cc2F)c2c(=O)n(C3CC3)c(=O)n(-c3cccc(NC(=O)CCOCCOCCOCCN=[N+]=[N-])c3)c12]

### Structure XXI: Chemical Structure of Pimasertib Analogue

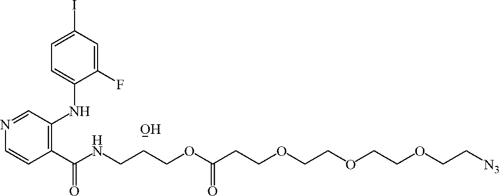

### Glutaminase Inhibitors

In some embodiments, the triantennary-GalNAc modified dendrimers are complexed with or conjugated to one or more inhibitors of glutaminase. Glutaminase (GLS), which is responsible for the conversion of glutamine to glutamate, plays a vital role in up-regulating cell metabolism for tumor cell growth. Exemplary glutaminase inhibitors include Bis-2-(5-phenylacetimido-1,2,4-thiadiazol-2-yl)ethyl sulfide (BPTES), 6-diazo-5-oxo-L-norleucine (DON), azaserine, acivicin, and CB-839. In some embodiments, the glutaminase inhibitors are BPTES analogs such as JHU-198, JHU-212, and JHU-329 (Thomas AG et al., Biochem Biophys Res Commun. (2014); 443(1): 32-36).

In some embodiments, dendrimers are conjugated to one or more glutaminase inhibitors. Exemplary glutaminase inhibitors include BPTES, DON, azaserine, acivicin, CB-839, JHU-198, JHU-212, and JHU-329. The glutaminase inhibitors can be functionalized, for example, with ether, ester, or amide linkage, optionally with one or more spacers/linkers, for ease of conjugation with the dendrimers and/or for desired release kinetics. In preferred embodiments, the glutaminase inhibitors or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). In preferred embodiments, dendrimers are conjugated to CB-839, or a derivative, analog or prodrug, or a pharmacologically active salt thereof. CB-839 has the following structure:

### Structure XXII: Chemical Structure of CB-839

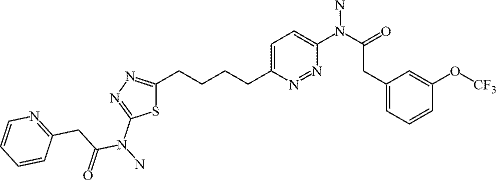 [SMILES: NN(C(=O)Cc1cccc(OC(F)(F)F)c1)c1ccc(CCCCc2nnc(N(N)C(=O)Cc3ccccn3)s2)nn1]

In some embodiments, dendrimers are conjugated to glutamine analog or antagonist L-[αS,5S]-α-amino-3-chloro-4,5-dihydro-5-isoxazoleacetic acid (acivicin), or a derivative, analog or prodrug, or a pharmacologically active salt thereof. Chemical structure of Acivicin is shown below in Structure XXIII.

### Structure XXIII

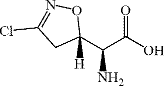 [SMILES: N[C@H](C(=O)O)[C@@H]1CC(Cl)=NO1]

Acivicin has been the subject of clinical trials for the treatment of cancer. Dosages and formulations are known in the art, see, for example, Hidalgo, Clinical Cancer Research, 4(11): 2763-2770 (1998), U.S. Pat. Nos. 3,856,807, 3,878,047, and 5,087,639. In one embodiment, dendrimers are conjugated to acivicin. In preferred embodiments, acivicin is functionalized, for example with ether, ester, N-alkyl, or amide linkage, optionally with one or more spacers/linkers such as polyethylene glycol (PEG), prior to conjugation to dendrimers.

### TIE II Antagonists

In some embodiments, the triantennary-GalNAc modified dendrimers are complexed with or conjugated to one or more antagonists of TIE II. Angiopoietin-1 receptor also known as CD202B (cluster of differentiation 202B) is a protein that in humans is encoded by the TEK gene. Also known as TIE2, it is an angiopoietin receptor. The angiopoietins are protein growth factors required for the formation of blood vessels (angiogenesis), which supports tumor growth and development. Therefore, in some embodiments, dendrimers are conjugated to one or more TIE II antagonists.

The TIE II antagonists can be functionalized, for example, with ether, ester, or amide linkage, optionally with one or more spacers/linkers, for ease of conjugation with the dendrimers and/or for desired release kinetics. The chemical structure of an exemplary TIE II inhibitor is shown below as Structure XXIV. TIE II inhibition of the free TIE II antagonist has a dissociation constant, K_{d}, about 8.8 nm and the TIE II inhibition of dendrimer conjugated TIE II antagonist (Structure XXIV) has a dissociation constant, K_{d}, about 25 nm. Thus, in preferred embodiments, TIE II antagonists are conjugated to dendrimers with or without a spacer in such a way that it minimizes the reduction in TIE II inhibition, for example, less than 1-fold, 2-fold, 3-fold, 4-fold, 5-fold, 10-fold, 20-fold, 30-fold, 40-fold, 50-fold, and 100-fold.

### Structure XXIV: TIE II Antagonist 1

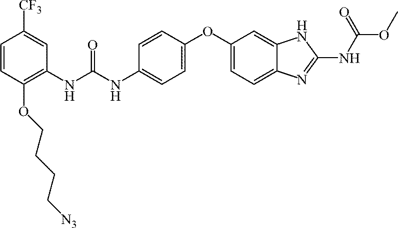 [SMILES: COC(=O)Nc1nc2ccc(Oc3ccc(NC(=O)Nc4cc(C(F)(F)F)ccc4OCCCCN=[N+]=[N-])cc3)cc2[nH]1]

In some embodiments, the dendrimers are complexed with or conjugated to two or more different classes of active agents, providing simultaneous delivery with different or independent release kinetics at the target site. In one embodiment, a generation 4 or generation 6 PAMAM dendrimer is conjugated to a TIE II inhibitor and gemcitabine, or analogs thereof. In another embodiment, a generation 4 or generation 6 PAMAM dendrimer is conjugated to a TIE II inhibitor and capecitabine, or analogs thereof. Exemplary synthesis routes of dendrimers conjugated to two or more different classes of active agents are shown in FIGS. 13A-13C.

### CXCR2 Inhibitors

In some embodiments, the triantennary-GalNAc modified dendrimers are complexed with or conjugated to one or more inhibitors of CXCR2. CXCR2 is expressed by many tumor cells and is involved in the chemotherapy resistance in different preclinical models of cancer (Poeta VM et al., Front Immunol. 2019; 10: 379). In breast cancer cells, CXCR2 deletion resulted in better response to Paclitaxel. In a melanoma model, the CXCR2 inhibitor Navarixin synergized with MEK inhibition whereas, in an ovarian tumor model, the CXCR2 inhibitor SB225002 improved the antiangiogenic therapy Sorafenib. In human gastric cancer, Reparixin, a CXCR1 and CXCR2 inhibitor, enhanced the efficacy of 5-fluorouracil.

CXCR2 targeting also inhibits tumor growth because it affects myeloid cell infiltration. In pancreatic tumors, CXCR2 inhibition prevented the accumulation of neutrophils unleashing the T cell response, resulting in inhibition of metastatic spreading and improved response to anti-PD-1. Interestingly, the combined treatment of CXCR2 and CCR2 inhibitors limited the compensatory response of TAMs, increased antitumor immunity and improved response to FX. Finally, in a prostate cancer model, CXCR2 inhibition by SB265610, decreased recruitment of myeloid cells and enhanced Docetaxel-induced senescence, limiting tumor growth.

Thus, in some embodiments, dendrimers are associated with or conjugated to one or more CXCR2 inhibitors. Exemplary CXCR2 inhibitors include Navarixin, SB225002, SB332235, SB265610, Reparixin, and AZD5069. In preferred embodiments, dendrimers are conjugated to Navarixin, SB225002, or SB332235, or a derivative, analog or prodrug, or a pharmacologically active salt thereof. The CXCR2 inhibitors can be functionalized, for example, with ether, ester, N-alkyl, or amide linkage, for ease of conjugation with the dendrimers and/or for desired release kinetics. In some embodiments, the CXCR2 inhibitors are conjugated to the dendrimers via N-alkyl linkage using click chemistry.

### CD73 Inhibitors

In some embodiments, the triantennary-GalNAc modified dendrimers are complexed with or conjugated to one or more inhibitors of CD73. CD73 converts extracellular adenosine monophosphate (AMP) into immunosuppressive adenosine, which shuts down anti-tumor immune surveillance at the level of T cells and natural killer (NK) cells, dendritic cells (DCs), myeloid-derived suppressor cells (MDSCs), and tumor associated macrophages (TAMs). In cancer, upregulation of CD73 expression in tumor cells and cells in the tumor stroma results in an increase in adenosine production, which leads to inhibition of T cell and NK cell cytotoxicity, cytokine production and proliferation as well as suppression of antigen-presenting cells (APCs,; enhanced regulatory T cell (Treg) proliferation and suppressive activity, and MDSCs and macrophage M2 polarization. These changes enable tumor growth and disease progression.

Thus, in some embodiments, dendrimers are conjugated to one or more CD73 inhibitors. Exemplary CD73 inhibitors include non-hydrolyzable AMP analogs such as adenosine 5′-(α,β-methylene)diphosphate (APCP), flavonoid-based compounds such as quercetin, and purine nucleotide analogs such as tenofovir and sulfonic acid compounds. In preferred embodiments, dendrimers are conjugated to one or more CD73 inhibitors including APCP, quercetin, or tenofovir, or a derivative, analog or prodrug, or a pharmacologically active salt thereof. The CD73 inhibitors can be functionalized, for example with ether, ester, or amide linkage, optionally with one or more spacers/linkers, for ease of conjugation with the dendrimers and/or for desired release kinetics. In preferred embodiments, the CD73 inhibitors or derivatives, analogs or prodrugs thereof, are conjugated to the dendrimers via Cu (I) catalyzed alkyne-azide click or thiol-ene click chemistry.

In some embodiments, one or more CD73 inhibitors and/or derivatives or analogs thereof having structures as shown in Structure XXV a-i and Structure XXVI a-c below are suitable for conjugation to dendrimers.

### Structure XXV A-I: Structures of CD73 Inhibitors and Analogs Thereof

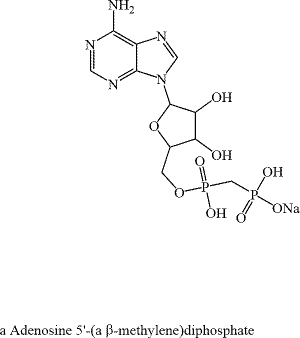

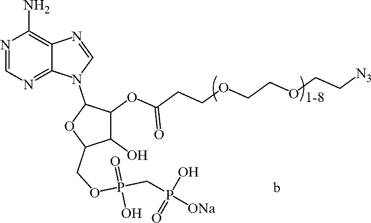

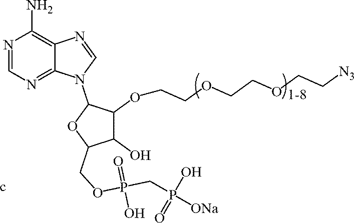

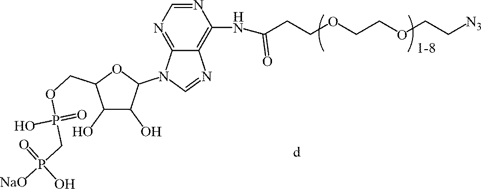

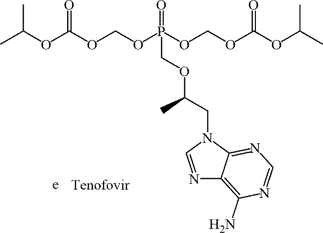

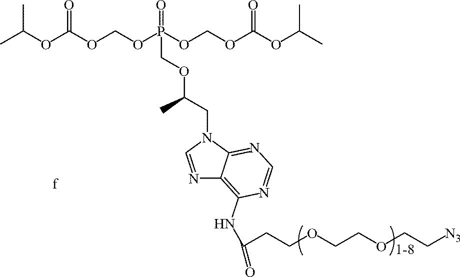

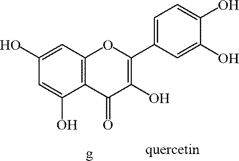

[symbol]

indicates text missing or illegible when filed

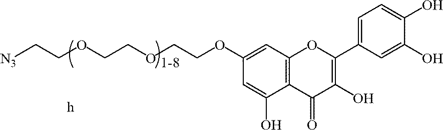

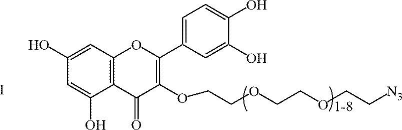

### Structure XXVI A-C: Structures of CD73 Inhibitors and Analogs Thereof

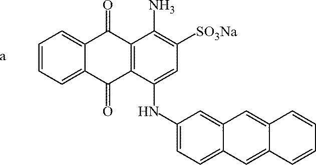

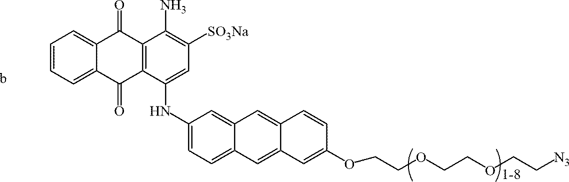

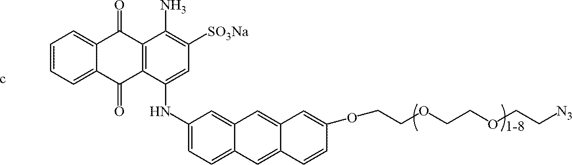

### Arginase Inhibitors

In some embodiments, the triantennary-GalNAc modified dendrimers are complexed with or conjugated to one or more arginase inhibitors. Expression of the enzyme arginase 1 (Arg1) is a defining feature of immunosuppressive myeloid cells and leads to depletion of L-arginine, a nutrient required for T cell and natural killer (NK) cell proliferation. Blocking Arg1 activity in the context of cancer could therefore shift the balance of L-arginine metabolism to favor lymphocyte proliferation. Indeed, in murine studies, injection of the arginase inhibitor nor-NOHA or genetic disruption of Arg1 in the myeloid compartment resulted in reduced tumor growth, indicating that Arg1 is pro-tumorigenic.

Thus, in some embodiments, dendrimers are associated with or conjugated to one or more arginase inhibitors. In some embodiments, one or more arginase inhibitors are boronic acid-based arginase inhibitors, for example, derivatives of 2—(S)—amino-6-boronohexanoic acid (ABH) (Borek B et al., Bioorg Med Chem. 2020 Sep 15;28(18):115658), or derivatives, analogs or prodrugs, or pharmacologically active salts thereof. In preferred embodiments, dendrimers are conjugated to one or more arginase inhibitors or derivatives, analogues or prodrugs, or pharmacologically active salts thereof. Arginase inhibitors can be functionalized, for example with ether, ester, amine, or amide linkage, optionally with one or more spacers/linkers, for ease of conjugation with the dendrimers and/or for desired release kinetics. In preferred embodiments, arginase inhibitors or derivatives, analogs or prodrugs thereof, are conjugated to the dendrimers via Cu (I) catalyzed alkyne-azide click or thiol-ene click chemistry.

In some embodiments, one or more arginase inhibitors and/or derivatives or analogs thereof having structures as shown in Structure XXVII a-g and Structure XXVIII a-h below are conjugated to dendrimers.

### Structure XXVII A-G: Structures of Arginase Inhibitors and Analogs Thereof

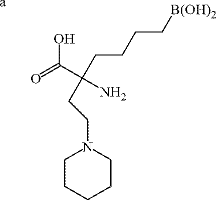

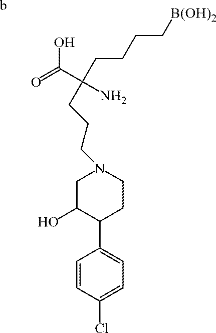

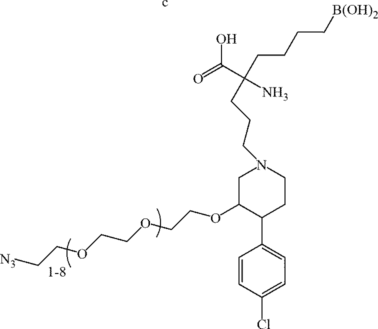

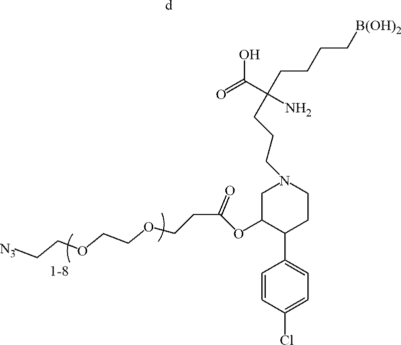

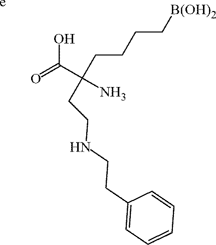

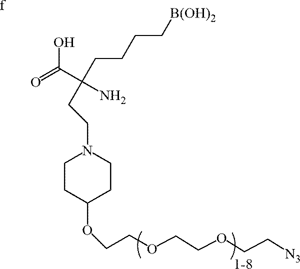

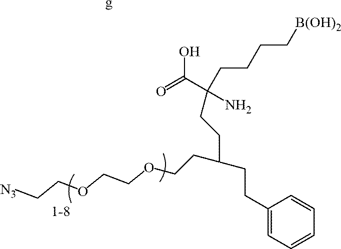

### Structure XXVIII A-H: Structures of Arginase Inhibitors and Analogs Thereof

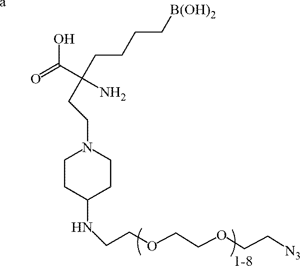

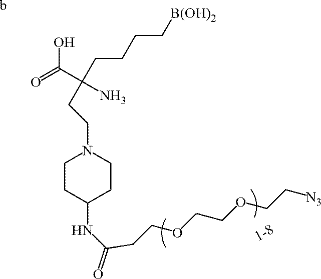

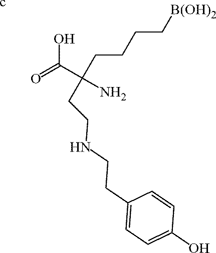

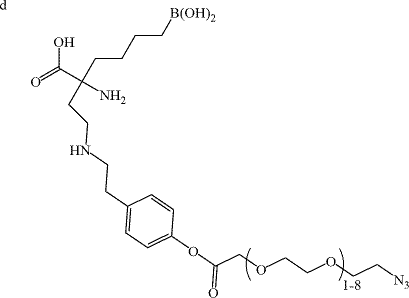

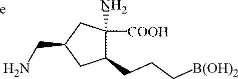

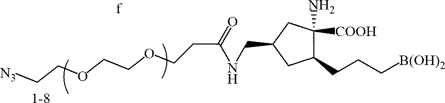

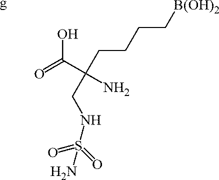

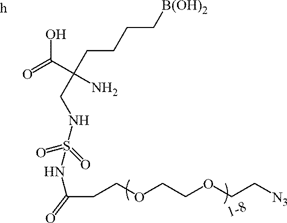

### Phosphatidylinositol-3-Kinase (PI3K) Inhibitors

In some embodiments, the triantennary-GalNAc modified dendrimers are complexed with or conjugated to one or more inhibitors of PI3K. Dysregulation of PI3K/PTEN pathway components, resulting in hyperactivated PI3K signaling, is frequently observed in various cancers and correlates with tumor growth and survival. Resistance to a variety of anticancer therapies, including receptor tyrosine kinase (RTK) inhibitors and chemotherapeutic agents, has been attributed to the absence or attenuation of downregulating signals along the PI3K/PTEN pathway. Macrophage PI 3-kinase γ controls a critical switch between immune stimulation and suppression during inflammation and cancer. PI3Kγ signaling through Akt and mTor inhibits NFκB activation while stimulating C/EBPβ activation, thereby inducing a transcriptional program that promotes immune suppression during inflammation and tumor growth. By contrast, selective inactivation of macrophage PI3Kγ stimulates and prolongs NFκB activation and inhibits C/EBPβ activation, thus promoting an immunostimulatory transcriptional program that restores CD8+ T cell activation and cytotoxicity.

Thus, in some embodiments, dendrimers are associated with or conjugated to one or more PI3K inhibitor. In preferred embodiments, dendrimers are associated with or conjugated to one or more PI3K γ inhibitors. Exemplary PI3K inhibitors include BYL719 (alpelisib), INK1117 (serabelisib, MLN-1117 or TAK-117), XL147 (SAR245408), pilaralisib, WX-037, NVP-BEZ235 (dactolisib or BEZ235), LY3023414 (prexasertib), XL765 (voxtalisib or SAR245409), PX-866, ZSTK474, NVP-BKM120 (buparlisib), GDC-0941(pictilisib), and BAY80-6946 (copanlisib). The PI3K inhibitors can be functionalized, for example with ether, ester, or amide linkage, optionally with one or more spacers/linkers, for ease of conjugation with the dendrimers and/or for desired release kinetics. In preferred embodiments, the PI3K inhibitors or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). The chemical structure of exemplary PI3K inhibitors is shown below as Structure XXIX and Structure XXX.

### Structure XXIX A-K: Structures of PI3K Inhibitors and Analogs Thereof

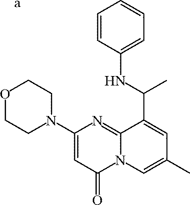

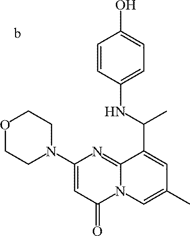

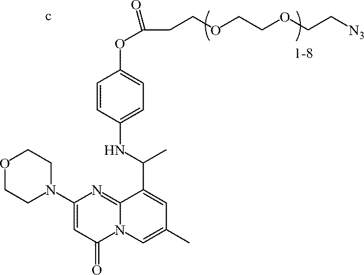

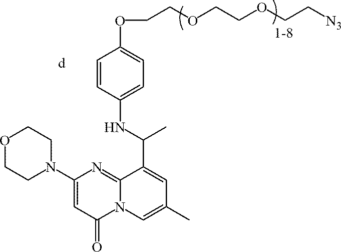

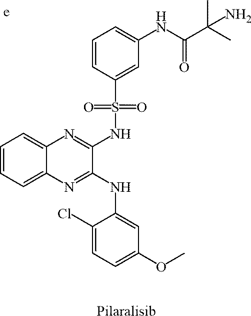

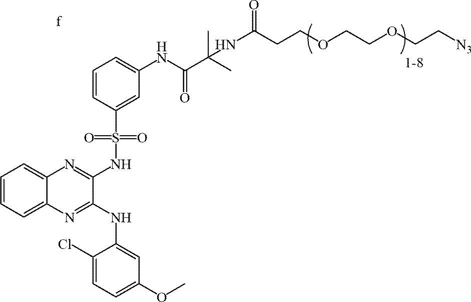

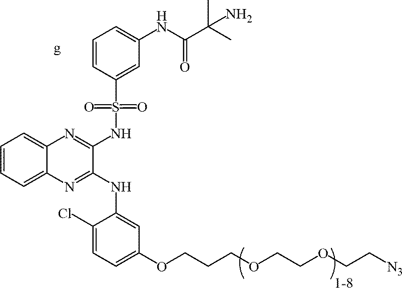

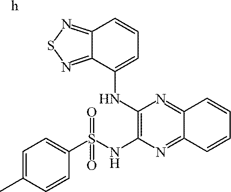

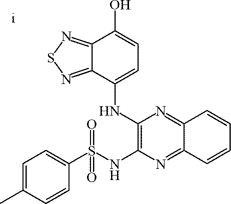

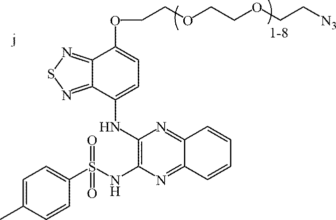

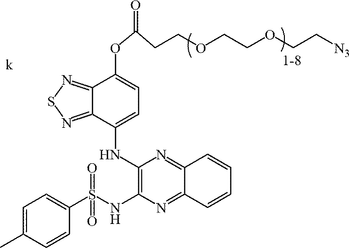

### Structure XXX A-F: Structures of PI3K Inhibitors and Analogs Thereof

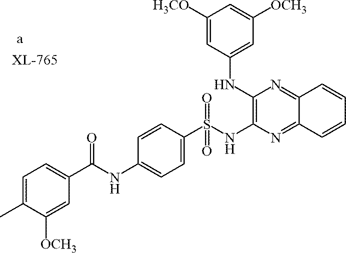

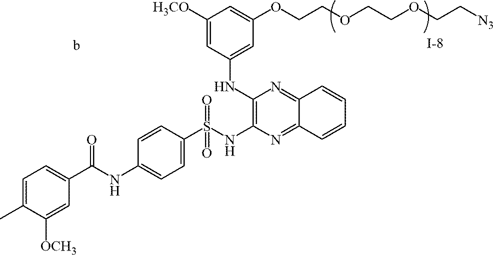

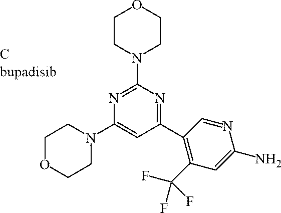

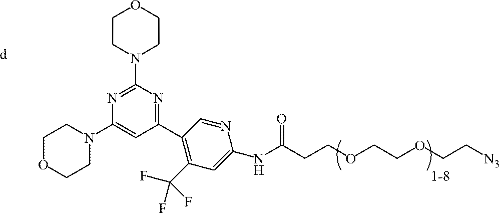

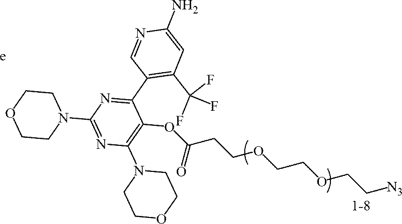

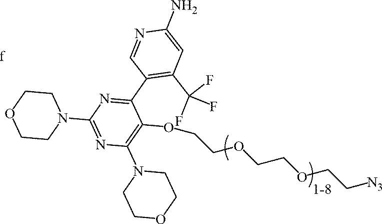

### Toll-Like Receptor 4 (TLR4) and TLR7 Agonists

In some embodiments, the triantennary-GalNAc modified dendrimers are complexed with or conjugated to one or more Toll-like Receptor 4 (TLR4) agonists and/or Toll-like Receptor 7 (TLR7) agonists. TLRs play a vital role in activating immune responses. TLRs recognize conserved pathogen-associated molecular patterns (PAMPs) expressed on a wide array of microbes, as well as endogenous DAMPs released from stressed or dying cells.

In some embodiments, dendrimers are associated with or conjugated to one or more TLR4 agonists. Exemplary TLR4 agonists include synthetic toll-like receptor 4 agonist glucopyranosyl lipid A, Bacillus Calmette-Guérin (BCG) and monophosphoryl lipid A (MPLA). The TLR4 agonists can be functionalized, for example, with ether, ester, or amide linkage, optionally with one or more spacers/linkers, for ease of conjugation with the dendrimers and/or for desired release kinetics. In some embodiments, the dendrimers are generation 4, 5, or 6 hydroxyl-terminated PAMAM dendrimers. In preferred embodiments, the TLR4 agonists or derivatives, analogues or prodrugs thereof, are conjugated to dendrimers via Cu (I) catalyzed alkyne-azide click or thiol-ene click chemistry, optionally via one or more spacers/linkers such as polyethylene glycol (PEG). Exemplary TLR4 agonists or analogues thereof are shown below.

### Structure XXXI A-B: Structures of Two TLR4 Agonist Analogues

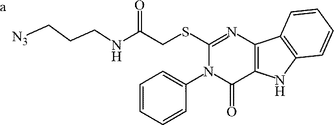

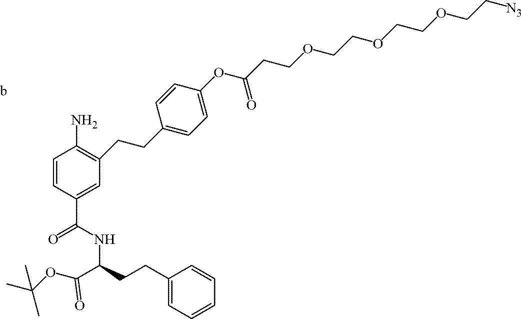

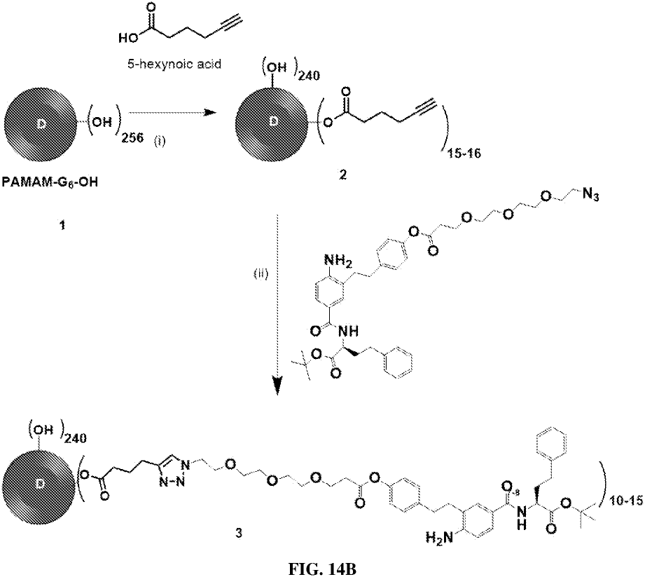

The chemical synthesis routes of exemplary TLR4 agonists conjugated to dendrimers are shown in FIGS. 14A and 14B.

In some embodiments, dendrimers are associated with or conjugated to one or more TLR7 agonists. Exemplary TLR7 agonists include imiquimod, resiquimod, gardiquimod, 852A, Loxoribine, Bropirimine, 3M-011, 3M-052, DSR-6434, DSR-29133, SC1, SZU-101, SM-276001, and SM -360320. In preferred embodiments, the TLR agonist is resiquimod. The TLR7 agonists can be functionalized, for example, with ether, ester, or amide linkage, optionally with one or more spacers/linkers, for ease of conjugation with the dendrimers and/or for desired release kinetics.

In some embodiments, dendrimers associated with or conjugated to one or more TLR4 or TLR7 agonists are used in combination with anti-tumor vaccines and/or adoptive cell therapy (ACT) as an adjuvant, for example to increase expression of innate immune genes, infiltration and expansion of activated effector T cells, antigen presentation, and durable immune responses.

### SHP2 Inhibitors

In some embodiments, the triantennary-GalNAc modified dendrimers are complexed with or conjugated to one or more SHP2 inhibitors. SHP2 (Src homology-2 domain-containing protein tyrosine phosphatase-2) is a non-receptor protein tyrosine phosphatase that removes tyrosine phosphorylation. Functionally, SHP2 serves as an important hub to connect several intracellular oncogenic signaling pathways, such as Jak/STAT, PI3K/AKT, RAS/Raf/MAPK, and PD-1/PD-L1 pathways. Mutations and/or overexpression of SHP2 has been associated with genetic developmental diseases and cancers.

Hence, in some embodiments, dendrimers are associated with or conjugated to one or more SHP2 inhibitors, or derivatives, analogs or prodrugs, or pharmacologically active salts thereof. Exemplary SHP2 inhibitors include inhibitors targeting the catalytic site and inhibitors targeting the allosteric site of SHP2, for example, TNO155, RMC-4630, JAB-3068, JAB-3312, and RMC-4550. SHP2 inhibitors can be functionalized, for example with ether, ester, or amide linkage, optionally with one or more spacers/linkers, for ease of conjugation with the dendrimers and/or for desired release kinetics. In some embodiments, the dendrimers are generation 4, 5, or 6 hydroxyl-terminated PAMAM dendrimers. In preferred embodiments, the SHP2 inhibitors or derivatives, analogs or prodrugs thereof, are conjugated to dendrimers via Cu (I) catalyzed alkyne-azide click or thiol-ene click chemistry, optionally via one or more spacers/linkers such as polyethylene glycol (PEG). Exemplary SHP2 inhibitors or analogues thereof are shown below.

### Structure XXXII A-B: Structures of Two SHP2 Inhibitor Analogues

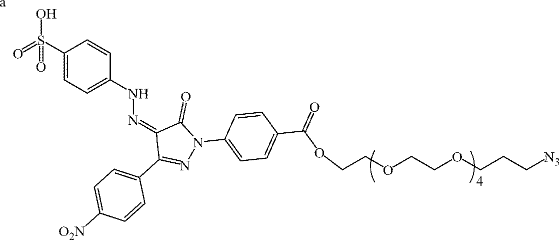

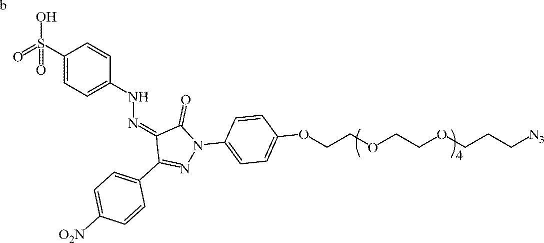

Some exemplary immunomodulatory agents used with dendrimers also include STING antagonists, JAK1 inhibitors, and anti-inflammatory agents. In preferred embodiments, dendrimers associated with or conjugated to one or more immunomodulatory agents including STING antagonists, JAK1 inhibitors, and anti-inflammatory agents are particularly suited for targeting one or more pro-inflammatory immune cells.

### 7. Additional Agents for Liver Cancer

In some embodiments, triantennary-β-GalNAc modified dendrimers are complexed, covalently conjugated, or intra-molecularly dispersed or encapsulated with one or more additional therapeutic agents including conventional cancer therapeutics such as chemotherapeutic agents, cytokines, chemokines, and radiation therapy. The majority of chemotherapeutic drugs can be divided into alkylating agents, antimetabolites, anthracyclines, plant alkaloids, topoisomerase inhibitors, and other antitumour agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In some embodiments, triantennary-GalNAc modified dendrimers complexed to, covalently conjugated to, or having intra-molecularly dispersed or encapsulated therein one or more chemotherapeutic agents. Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In one embodiment, triantennary-GalNAc modified dendrimers are covalently conjugated to capecitabine, preferably via an ester, ether, or amide linakge via a spacer such as PEG.

In another embodiment, triantennary-GalNAc modified dendrimers are covalently conjugated to gemcitabine, preferably via an ester, ether, or amide linakge via a spacer such as PEG.

In some embodiments, the active agents are histone deacetylase (HDAC) inhibitors. In one embodiment, the active agent is vorinostat. In other embodiments, the active agents are topoisomerase I and/or II inhibitors. In a particular embodiment, the active agent is etoposide or camptothecin.

Additional anti-cancer agents include, but are not limited to, irinotecan, exemestane, octreotide, carmofur, clarithromycin, zinostatin, tamoxifen, tegafur, toremifene, doxifluridine, nimustine, vindensine, nedaplatin, pirarubicin, flutamide, fadrozole, prednisone, medroxyprogesterone, mitotane, mycophenolate mofetil, and mizoribine.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

In some cases, the active agent is an anti-infectious agent. Exemplary anti-infectious agents include antiviral agents, antibacterial agents, antiparasitic agents, and anti-fungal agents. Exemplary antibiotics include moxifloxacin, ciprofloxacin, erythromycin, levofloxacin, cefazolin, vancomycin, tigecycline, gentamycin, tobramycin, ceftazidime, ofloxacin, gatifloxacin; antifungals: amphotericin, voriconazole, natamycin.

Any of the additional active compounds can be functionalized, for example with ether, ester, ethyl, or amide linkage, optionally with one or more spacers/linkers, for ease of conjugation with the dendrimers and/or for desired release kinetics. In preferred embodiments, active agents or derivatives, analogs or prodrugs thereof, are conjugated to the dendrimers via Cu (I) catalyzed alkyne-azide click or thiol-ene click chemistry, optionally via one or more spacers/linkers such as polyethylene glycol (PEG). In some embodiments, the additional active agents are chemotherapeutic agents or derivatives, analogs or prodrugs, or pharmacologically active salts thereof. In one embodiment, the active agent complexed or conjugated to dendrimer is methotrexate, or a derivative, analog or prodrug, or a pharmacologically active salt thereof, for example as shown in Structure XXXIII.

### Structure XXXIII: Chemical Structure of Methotrexate Analogue

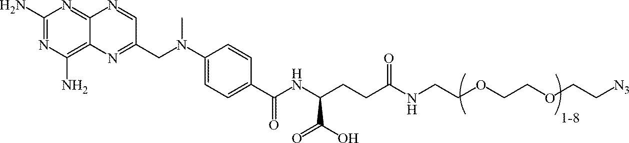

### 8. Agents for Treatment of Hypertension and Other Disorders

In some embodiments, the dendrimers are used to deliver one or more additional active agents, particularly one or more therapeutic, prophylactic and/or diagnostic agents to prevent or treat one or more symptoms of liver injuries and/or associated diseases or conditions such as infections, sepsis, diabetic complications, hypertension, obesity, high blood pressure, heart failure, kidney diseases, and cancers.

In some embodiments, other agents can be incorporated such as chemotherapeutic, antiangiogenic agents, and anti-excitotoxic agents such as valproic acid, D-aminophosphonovalerate, D-aminophosphonoheptanoate, inhibitors of glutamate formation/release such as baclofen, NMDA receptor antagonists, ranibizumab, and anti-VEGF agents including aflibercept, and immunomodulators such as rapamycin.

Other therapeutic agents that may be delivered include insulin sensitizer, pioglitazone.

In some embodiments, the active agent is an anti-infectious agent. Exemplary anti-infectious agents include antiviral agents, antibacterial agents, antiparasitic agents, and anti-fungal agents.

### 9. Diagnostic Agents

In some cases, the agent may include a diagnostic agent. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radiopaque. Dendrimer complexes can further include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes.

In further embodiments, a singular dendrimer complex composition can simultaneously treat and/or diagnose a disease or a condition at one or more locations in the body.

### III. Pharmaceutical Formulations

Pharmaceutical compositions including dendrimers and one or more active agents such as one or more angiotensin II receptor blockers may be formulated in a conventional manner using one or more physiologically acceptable carriers including excipients and auxiliaries which facilitate processing of the active compounds into preparations which can be used pharmaceutically. Formulation is dependent upon the route of administration chosen. In preferred embodiments, the compositions are formulated for parenteral delivery. In some embodiments, the compositions are formulated for subcutaneous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more dendrimer complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions. See, for example, Remington’s Pharmaceutical Sciences, 20th ed., Lippincott Williams & Wilkins, Baltimore, MD, 2000, p. 704.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

Pharmaceutical compositions formulated for administration by parenteral (intramuscular, intraperitoneal, intravenous or subcutaneous injection) and enteral routes of administration are described.

### A. Parenteral Administration

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intraarticular, subcapsular, subarachnoid, intraspinal and intrastemal injection and infusion. The dendrimers can be administered parenterally, for example, by subdural, intravenous, intrathecal, intraventricular, intraarterial, intra-amniotic, intraperitoneal, or subcutaneous routes. In preferred embodiments, the dendrimer compositions are administered via subcutaneous injection.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer’s dextrose, dextrose and sodium chloride, lactated Ringer’s and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer’s dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer’s dextrose, dextrose and sodium chloride, lactated Ringer’s and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer’s dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### IV. Methods of Making

### A. Methods of Making Dendrimers

Dendrimers can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using different methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward building inward and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or active agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20;20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of active agents are linked to one type of dendron and a different type of active agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in WO 2009/046446, WO 2015168347, WO 2016025745, WO 2016025741, WO 2019094952, and U.S. Pat. No. 8,889,101.

### B. Conjugating Triantennary N-Acetylgalactosamine (GalNAc) to Dendrimers

In some embodiments, the P-GalNAc-triantennary-PEG3-Azide is prepared as shown in FIG. 1. In some embodiments, a triantenary building block is prepared where three molecules of β-GalNAc-azide optionally with a linker such as PEG are grafted on a propargylated pentaerythritol building block to yield AB₃ type orthogonal building block. In other embodiments, an AB₄ monomer such as pentaerythritol or a derivative thereof is used as a core for conjugating to three molecules of β-GalNAc. In some embodiments, synthesis starts with the glycosylation reaction of β-D-GalNAc pentacetate (e.g., compound 1 of FIG. 1) with 2-[2-(2-azidoethoxy)ethoxy]ethan-1-ol (compound 2 of FIG. 1) to yield peracetylated β-GalNAc-azide with a PEG spacer/linker (e.g., compound 3 of FIG. 1). In some embodiments, pentaerythritol (compound 4) is selectively modified with three propargyl arms to yield tripropargyl pentaerythritol (e.g., compound 5 of FIG. 1). In some embodiments, the remaining one hydroxyl group on tripropargyl pentaerythritol is reacted with bis-chlorotetraethylene glycol (compound 7) to yield intermediate compound, an AB₃ building block (e.g., compound 8 of FIG. 1). In some embodiments, peracetylated β-GalNAc-PEG3-Azide is clicked with AB₃ building block (e.g., compound 8) using conventional CuAAC click reaction conditions to yield compound 9. In some embodiments, the success of click reaction is confirmed by ¹H NMR, HRMS and HPLC. In some embodiments, terminal chloride group of compound 9 is exchanged to azide by nucleophilic substitution to yield compound 10. In some embodiments, the last step is the transesterification to yield deacetylated P-GalNAc-triantetennary-PEG3 azide (compound 11) building block, a GalNAc dendron.

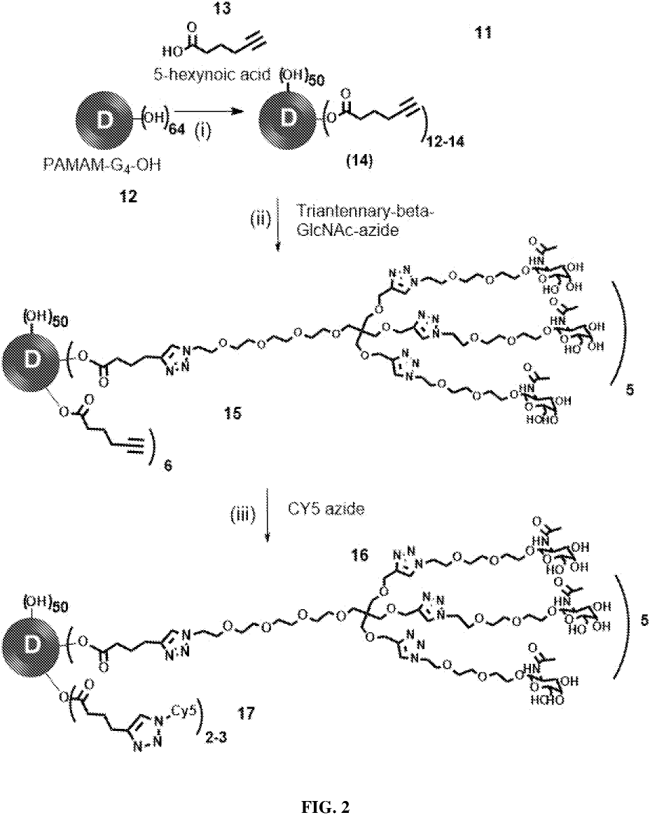

In some embodiments, the P-GalNAc-triantennary-PEG3-Azide is conjugated to a dendrimer as shown in FIG. 2. In some embodiments, generation 4 or generation 6 hydroxyl terminated PAMAM dendrimer undergoes partial esterification with 5-hexynoic acid to yield a compound with two or more hexyne arms, preferably 5 to 20, or 10 to 15, or 12 to 14 hexyne arms, attached to the dendrimer. In some embodiments, one or more P-GalNAc-triantennary-PEG3-Azide is conjugated to dendrimer having hexyne arms attached thereto using copper catalyzed click (CuAAc) reaction to yield P-GalNAc-triantennary modified dendrimers. In preferred embodiments, one or more hexyne arms conjugated to the dendrimer are for conjugation to the GalNAc dendron or β-GalNAc-triantennary-PEG3-Azide, and one or more hexyne arms conjugated to the dendrimer are for conjugation to drugs or imaging agents. In one embodiment, 5-6 hexyne arms are for conjugation to the GalNAc dendron or β-GalNAc-triantennary-PEG3-Azide and 5-7 hexyne arms are for conjugation to drugs and/or imaging agents. Introduction of 5-6 arms of dendron results in 15-18 GalNAc units on the final structure.

### V. Methods of Use

Methods of selective delivery of active agents to hepatocytes are provided. It has been established that triantennary-β-GalNAc modified dendrimer compositions selectively bind to asialoglycoprotein receptors (ASGPR) on hepatocyte cells. The efficient binding to the ASGPR receptors directs selective internalization of the dendrimer-triantennary-β-GalNAc within the hepatocyte via receptor-mediated endocytosis. The low pH in the endosome within the hepatocyte cells results in the disruption of the interactions between the triantennary-β-GalNAc ligand and the ASGPR receptor, causing release of the ligand into the hepatocytes. Methods of using the triantennary-β-GalNAc modified dendrimer compositions for selective delivery, accumulation, and intracellular release of one or more active agents to hepatocytes are described.

### A. Methods for Treating Liver Disorders and Diseases

Methods of using dendrimer-triantennary GalNAc modified compositions for treating or preventing one or more liver diseases or disorders in a subject are described.

Dendrimer-triantennary GalNAc compositions including one or more active agents to treat or prevent a liver disease or disorder can be administered to a subject to treat, prevent, and/or diagnose one or more symptoms of one or more liver disorders and/or diseases in the subject. The methods can include the step of identifying and/or selecting a subject in need thereof.

Methods for treating or preventing one or more symptoms of one or more liver disorders and/or diseases include administering to the subject dendrimers complexed, covalently conjugated, or intra-molecularly dispersed or encapsulated with one or more therapeutic or prophylactic agents, in an amount effective to treat, alleviate or prevent one or more symptoms of one or more liver disorders and/or diseases. In preferred embodiments, the dendrimer compositions including one or more anti-oxidant agents and/or angiotensin II type I receptor blockers, or formulations thereof are administered in an amount effective to treat or prevent one or more symptoms of one or more liver disorders and/or diseases, for example, reducing lobular inflammation in the liver.

In one embodiment, methods for treating or preventing one or more liver disorders and/or diseases include administering to the subject compositions including triantennary-β-GalNAc modified hydroxyl terminated PAMAM dendrimers of generation 4, generation 5, generation 6, generation 7, or generation 8 covalently conjugated to one or more angiotensin II type I receptor blockers, in an amount effective to treat or prevent one or more symptoms of one or more liver disorders and/or diseases.

### 1. Liver Disorders and Diseases to Be Treated

In some embodiments, triantennary-GalNAc modified dendrimers complexed with or conjugated to one or more active agents to treat, prevent, and/or diagnose one or more liver disorders and/or diseases are administered to a subject to treat, prevent, and/or diagnose one or more symptoms of one or more liver disorders and/or diseases in the subject.

Dendrimer-triantennary-β-GalNAc compositions are effective for treating or ameliorating one or more symptoms of a liver disease, or disorder, such as acute or chronic liver diseases. Exemplary indications that can be treated include, but are not limited to, acute liver failure (acute hepatitis, fulminant hepatitis), e.g., resulting from neoplastic infiltration, acute Budd-Chiari syndrome, heatstroke, mushroom ingestion, metabolic diseases such as Wilson’s disease, or associated with viral liver disease such as caused by herpes simplex viruses, cytomegalovirus, Epstein-Barr virus, parvoviruses, hepatitis viruses (e.g., hepatitis A, hepatitis E, hepatitis D+B infections), or drug-induced liver injury, including rifampicin-induced hepatotoxicity, acetaminophen-induced hepatotoxicity, recreational-drug induced toxicity such as by 3,4-methylenedioxy-N-methylamphetamine (MDMA, also known as ecstasy), or cocaine-induced toxicity, acute ischemic hepatocellular injury, or hypoxic hepatitis, or resulting from traumatic liver injury. The methods can treat and prevent any hyperacute, acute and subacute liver disease defined by the occurrence of encephalopathy, coagulopathy and jaundice in an individual with a previously normal liver.

Symptoms and clinical manifestations of acute liver disease include jaundice and encephalopathy, and impaired liver function (e.g., loss of metabolic function, decreased gluconeogenesis leading to hypoglycemia, decreased lactate clearance leading to lactic acidosis, decreased ammonia clearance leading to hyperammonemia, and reduced synthetic capacity leading to coagulopathy). Acute liver diseases and disorders are often associated with multiple systemic manifestations, including immunoparesis contributing to high risk of sepsis; systemic inflammatory responses, with high energy expenditure or rate of catabolism; portal hypertension; kidney dysfunction; myocardial injury; pancreatitis (particularly in acetaminophen-related disease); inadequate glucocorticoid production in the adrenal gland contributing to hypotension; and acute lung injury, leading to acute respiratory distress syndrome.

All the methods described can also include the step of identifying and selecting a subject in need of treatment, or a subject who would benefit from administration with the compositions. In some embodiments, the subject has been medically diagnosed as having an acute liver disease or disorder by exhibiting clinical (e.g., physical) symptoms of the disease. In other embodiments, the subject has been medically diagnosed as having a sub-acute or chronic liver disease or disorder by exhibiting clinical (e.g., physical) symptoms, which are indicative of an increased risk or likelihood of developing acute liver disease. Therefore, in some embodiments, formulations of the disclosed dendrimer compositions are administered to a subject prior to a clinical diagnosis of acute liver disease.

In preferred embodiments, the methods treat or prevent non-alcoholic steatohepatitis, liver fibrosis associated with non-alcoholic steatohepatitis, primary biliary cholangitis.

### I. Non-Alcoholic Fatty Liver Disease (NAFLD)

In some embodiments, dendrimer-triantennary-β-GalNAc compositions treat or alleviate one or more symptoms associated with nonalcoholic fatty liver disease (NAFLD). NAFLD represents a clinico-pathological spectrum of disease that primarily manifests as excessive accumulation of fat in the hepatocyte (steatosis). NAFLD encompasses the entire spectrum of diseases ranging from simple steatosis to non-alcoholic steatohepatitis (NASH), which can lead to life-threatening hepatic cirrhosis and hepatocellular carcinoma in its most severe form. It is considered to be the hepatic manifestation of the metabolic syndrome, whose other pathologies include obesity, insulin resistance, hypertension and hyperlipidemia. Histologically, NASH is characterized by hepatic steatosis and signs of intralobular inflammation with ballooning degeneration of the hepatocytes. The estimated prevalence of NASH is much lower than NAFLD and ranges from 3 to 5%. Twenty percent of NASH patients are reported to develop cirrhosis, and 30-40% of patients with NASH cirrhosis experience a liver related death.

In some embodiments, the dendrimer compositions are administered in an amount effective to prevent the transformation of NAFLD into NASH and to improve the pathophysiology of the disease.

NAFLD is broadly categorized into two phenotypes: non-alcoholic fatty liver (NAFL) which is marked by isolated steatosis, while the more aggressive subtype, non-alcoholic steatohepatitis (NASH), is characterized by cell injury, inflammatory cell infiltration and hepatocyte ballooning that may further progress to fibrosis, cirrhosis, and hepatocellular carcinoma (HCC). In some embodiments, the dendrimer compositions are used in an amount effective for treating or ameliorating one or more symptoms of non-alcoholic steatohepatitis (NASH).

Methods to treat and/or prevent one or more symptoms of NAFLD or NASH typically include administering to a subject in a need thereof an effective amount of a composition including triantennary-β-GalNAc modified hydroxyl terminated PAMAM dendrimers and one or more agents to treat and/or alleviate one or more symptoms associated with NAFLD or NASH. In one embodiment, the dendrimer compositions including triantennary-β-GalNAc modified hydroxyl terminated PAMAM dendrimers of generation 4, generation 5, or generation 6 covalently conjugated to one or more angiotensin II type I receptor blockers.

In some embodiments, the dendrimer-triantennary-β-GalNAc compositions are administered in an amount effective to inhibit or reduce serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality.

### II. Liver Cancer

In some embodiments, compositions of dendrimer-triantennary-β-GalNAc conjugated or complexed with one or more immunomodulatory agents, one or more chemotherapeutic agents, and/or additional therapeutic or diagnostic agents are administered to a subject having a proliferative disease, such as a benign or malignant tumor. In some embodiments, the subjects to be treated have been diagnosed with stage I, stage II, stage III, or stage IV cancer. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

In some embodiments, the dendrimer-triantennary-β-GalNAc compositions treat or alleviate one or more symptoms associated with liver cancer. In some embodiments, the subject has been medically diagnosed as having liver cancer.

In some embodiment, the dendrimer-triantennary-β-GalNAc compositions treat or alleviate one or more symptoms associated with hepatocellular carcinoma (HCC). HCC development results from the interaction between environmental and genetic factors. Liver cirrhosis, hepatitis B virus (HBV) and hepatitis C virus (HCV) infection, excessive alcohol consumption, ingestion of aflatoxin B1, and nonalcoholic steatohepatitis (NASH) are important risk factors for HCC development.

Methods to treat and/or prevent one or more symptoms of liver cancer typically include administering to a subject in a need thereof an effective amount of a composition including triantennary-β-GalNAc modified hydroxyl terminated PAMAM dendrimers and one or more agents to treat and/or alleviate one or more symptoms associated with liver cancer or HCC. In one embodiment, the dendrimer compositions including triantennary-β-GalNAc modified hydroxyl terminated PAMAM dendrimers of generation 4, generation 5, or generation 6 complexed, covalently conjugated, or intra-molecularly dispersed or encapsulated with one or more of STING agonists, CSF1R inhibitors, PARP inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, or combinations thereof.

In some embodiments, the dendrimer-triantennary-β-GalNAc compositions are administered in an amount effective to reduce the number and/or proliferation of cancer cells, reduce the tumor size, inhibit cancer cell infiltration into peripheral organs, inhibit tumor metastasis, inhibiting tumor growth, increase rates of long-term survival, improve response to immune checkpoint blockade, and/or induce immunological memory that protects against tumor re-challenge.

### 2. Dosages and Effective Amounts

Dosage and dosing regimens are dependent on the severity and location of the disorder or injury and/or methods of administration, and can be determined by those skilled in the art. A therapeutically effective amount of the dendrimer composition used in the treatment of liver disorders and/or diseases is typically sufficient to reduce or alleviate one or more symptoms of liver disorders and/or diseases.

Preferably, the active agents do not target or otherwise modulate the activity or quantity of healthy cells not within or associated with the diseased/damaged tissue, or do so at a reduced level compared to cells associated with the diseased/damaged liver. In this way, by-products and other side effects associated with the compositions are reduced.

A pharmaceutical composition including a therapeutically effective amount of the dendrimer compositions and a pharmaceutically acceptable diluent, carrier or excipient is described. In some embodiments, the pharmaceutical compositions include an effective amount of triantennary- GalNAc modified hydroxyl-terminated PAMAM dendrimers conjugated to telmisartan. In some embodiments, dosage ranges suitable for use are between about 0.1 mg/kg and about 100 mg/kg, inclusive; between about 0.5 mg/kg and about 40 mg/kg, inclusive; between about 1.0 mg/kg and about 20 mg/kg, inclusive; and between about 2.0 mg/kg and about 10 mg/kg, inclusive.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose. In some embodiments, the dosage unit suitable for use are (assuming the weight of an average adult patient is 70 kg) between 5 mg/dosage unit and about 7000 mg/ dosage unit, inclusive; between about 35 mg/ dosage unit and about 2800 mg/ dosage unit, inclusive; and between about 70 mg/ dosage unit and about 1400 mg/ dosage unit, inclusive; and between about 140 mg/ dosage unit and about 700 mg/ dosage unit, inclusive.

The actual effective amounts of dendrimer complex can vary according to factors including the specific active agent administered, the particular composition formulated, the mode of administration, and the age, weight, condition of the subject being treated, as well as the route of administration and the disease or disorder. The subjects are preferably humans. Generally, the dosage will be lower for intravenous injection or infusion compared to other systemic routes of administration such as oral and based on wt/patient as compared to topical, local or regional administration which will be based on the area to be treated.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

In some embodiments, dosages are administered once, twice, or three times daily, or less frequently, i.e., every other day, two days, three days, four days, five days, or six days to a human. In some embodiments, dosages are administered about once or twice every week, every two weeks, every three weeks, or every four weeks. In some embodiments, dosages are administered about once or twice every month, every two months, every three months, every four months, every five months, every six months, or less frequently.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### 3. Controls

The effect of the dendrimer compositions including one or more agents can be compared to a control or alternative treatment. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art. In some embodiments, an untreated control subject suffers from the same acute liver disease or condition as the treated subject.

### B. Combination Therapies and Procedures

The compositions can be administered alone or in combination with one or more conventional therapies. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of an agent targeting the site of treatment. The additional active agent(s) can have the same or different mechanisms of action. In some embodiments, the combination results in an additive effect on the treatment of the liver condition. In some embodiments, the combinations result in a more than additive effect on the treatment of the disease or disorder.

The additional therapy or procedure can be simultaneous or sequential with the administration of the dendrimer composition. In some embodiments, the additional therapy is performed between drug cycles or during a drug holiday that is part of the compositions dosage regime. For example, in some embodiments, the additional therapy or procedure is surgery, a radiation therapy, chemotherapy, liver transplant, stem cell transplantation, or mesenchymal stem cells (MSCs).

Exemplary additional therapies or procedures include lifestyle modification such as avoiding saturated fat, excessive sugar-containing diet, soft drinks, fast food, and refined carbohydrates and were also encouraged to perform moderate exercise. Diabetic patients can be treated with lifestyle modification and, if required, with oral sulphonylureas-gliclazide, glimeperide, and/or with insulin. Dyslipidemia can be managed with statin, and for hypertension, antihypertensive drugs.

In some embodiments, the compositions and methods are used prior to or in conjunction, subsequent to, or in alternation with treatment with one or more additional therapies or procedures. Additional therapeutic agents include conventional cancer therapeutics such as chemotherapeutic agents, cytokines, chemokines, and radiation therapy. The majority of chemotherapeutic drugs can be divided into alkylating agents, antimetabolites, anthracyclines, plant alkaloids, topoisomerase inhibitors, and other antitumour agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the compositions and methods are used prior to or in conjunction with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

Methods of adoptive T cell therapy are known in the art and used in clinical practice. Generally adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient’s blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol.,189(7):3299-310 (2012).

Historically, adoptive T cell therapy strategies have largely focused on the infusion of tumor antigen specific cytotoxic T cells (CTL) which can directly kill tumor cells. However, CD4+ T helper (Th) cells such as Th1, Th2, Tfh, Treg, and Th17 can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th₁ have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ- chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (05 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance body’s own anti-tumor immune functions.

In vivo efficacy study of these triantennary-GalNAc modified dendrimers can be assessed in mouse models of non-alcoholic Steatohepatitis, e.g., STAMTM Model (Mice) of non-alcoholic Steatohepatitis
Methods
   Pathogen-free 14 day-pregnant C57BL/6 mice can be obtained from Japan SLC, Inc.(Japan);
   NASH can be established in male mice by a single subcutaneous injection of 200 µg streptozotocin (STZ, Sigma, USA) 2 days after birth and feeding with a high fat diet (CLEA Japan Inc., Japan) ad libitum after 4 weeks of age (day 28 ± 2),
   NASH mice can be randomized into 8 groups of 8 mice and 2 groups of 4 mice at 6 weeks of age (day 42 ± 2) the day before the start of treatment based on their body weight,
   Littermate control mice without STZ priming (n=8) can be fed with normal diet ad libitum and set up for control purpose,
   If an animal shows >25% body weight loss within a week or >20% body weight loss compared to previous day, the animal will be euthanized ahead of study termination. If it shows a moribundity sign such as prone position, the animal will be euthanized ahead of study termination. The samples will not be collected from euthanized animals,
   Individual body weight will be measured daily during the treatment period,
   Survival, clinical signs and behavior of mice will be monitored daily,
Groups
   Group 1 (Normal): Eight normal mice will be fed with normal diet ad libitum without any treatment and sacrificed at 9 weeks of age,
   Group 2 (Vehicle): Eight NASH mice will be intraperitoneally administered vehicle [saline] in a volume of 10 mL/kg every other day from 6 to 9 weeks of age,
   Group 3 (Telmisartan): Eight NASH mice will be orally administered pure water supplemented with Telmisartan at a dose of 10 mg/kg once daily from 6 to 9 weeks of age,
   Group 4 (Obeticholic acid, or “OCA”): Eight NASH mice will be orally administered 1% methlycelluose supplemented with OCA at a dose of 30 mg/kg once daily from 6 to 9 weeks of age,
   Group 5 (Dendrimer-Triantenary-β-GlcNAc-azide-Telmisartan amide conjugate, or “D-Tel” high): Eight NASH mice will be intraperitoneally administered vehicle supplemented with D-Tel at a dose of 90 mg/kg every other day from 6 to 9 weeks of age,
   Group 6 (D-Tel low): Eight NASH mice will be intraperitoneally administered vehicle supplemented with D-Tel at a dose of 18 mg/kg every other day from 6 to 9 weeks of age,
   Group 7 (Dendrimer-Triantenary-β-GlcNAc-azide-Telmisartan ester conjugate, or “D-TelB” high): Eight NASH mice will be intraperitoneally administered vehicle supplemented with D-TelB at a dose of 90 mg/kg every other day from 6 to 9 weeks of age.
   Group 8 (D-OCA high): Eight NASH mice will be intraperitoneally administered vehicle supplemented with D-OCA at a dose of 315 mg/kg every other day from 6 to 9 weeks of age,
   Group 9 (D-OCA low): Eight NASH mice will be intraperitoneally administered vehicle supplemented with D-OCA at a dose of 63 mg/kg every other day from 6 to 9 weeks of age,
   Group 10 (D-Cy5-6 wks): Four NASH mice will be intraperitoneally administered vehicle supplemented with D-Cy5 at a dose of 50 mg/kg single shot at 6 weeks of age,
   Group 11 (D-Cy5-9 wks): Four NASH mice will be intraperitoneally administered vehicle supplemented with D-Cy5 at a dose of 50 mg/kg single shot at 9 weeks of age,
   Mice in group 10 and 11 will be sacrificed at 6 and 9 weeks of age 48 hours after the administration. Mice in group 1 - 9 will be sacrificed at 9 weeks of age for the following assay, group 10 and 11 will be sacrificed at 6 and 9 weeks of age for the following assays,
Measurement of organ weight:
   Individual liver weight will be measured,
   Liver-to-body weight ratio will be calculated,
Biochemical assays (group 1 - 9):
   Non-fasting serum ALT levels will be quantified by FUJI DRI CHEM (Fujifilm, Japan),
   Liver triglyceride will be quantified by Triglyceride E-test kit (FUJIFUILM Wako Pure Chemical Corporation, Japan),
Histological analyses for liver sections (according to a routine method) (group 1 - 9):
   HE staining and estimation of NAFLD Activity score,
   Sirius-red staining and estimation of the percentage of fibrosis area,
Sample collection and fixation:
   After completion of the in-life portion of the study, the following samples will be collected for further analyses or shipping,
   Animals in group 10-11 will be anesthesia with isoflurane and perfused with saline (followed by 4% neutral buffered formalin, NBF, pH 7.4) through left ventricle for 20-30 min. Dissect the animal and collect the tissue samples (right and left kidneys, liver) in sequential order. Sample thickness will be less than approximately 5 mm to ensure proper fixation. Trim a flat surface across the area of interest. Put the samples into 4% NBF for fixation immediately. Fix the samples in 4% NBF overnight at room temperature.

After fixation, the samples will be performed the following process.
Samples processing
   1. Place the tissues in PBS for 5 min x 3;
   2. Place the tissues in 10% sucrose (In PBS) for 24 hours at 4° C.;
   3. Place the tissues in 20% sucrose (In PBS) for 24 hours at 4° C.;
   4. Place the tissues in 30% sucrose (In PBS) for 24 hours at 4° C.;
   5. Place the tissues in 30% sucrose (In PBS): OCT (1:1) for 24 hours at 4° C.;
Procedures for tissue embedding
   Put the tissue and 30% sucrose:OCT (1:1-2) into the embedding module, adjust tissue orientation;
   Put the module on flat dry ice, waiting for solidification;
   Store the embedding model at -80° C.;
Sectioning
   Put embedding model on Thermo HM550 cryostat and sectioned axially into 10 µm thickness;
   Store the slides at -80° C. before use.
Samples
   Frozen serum samples (group 1 - 9),
   Frozen liver samples (group 1 - 11),
   Frozen liver section (group 10 - 11),
   O.C.T.-embedded liver blocks (group 10 - 11),
   O.C.T.-embedded kidney blocks (group 10 - 11),
Statistical tests (group 1 - 9)
   Statistical tests will be performed using Bonferroni Multiple Comparison Test. P values <0.05 will be considered statistically significant.

### V. Kits

The compositions can be packaged in kit. The kit can include a single dose or a plurality of doses of a composition including one or more active agents encapsulated in, associated with, or conjugated to a dendrimer, and instructions for administering the compositions. Specifically, the instructions direct that an effective amount of the composition be administered to an individual with a particular liver condition/disease as indicated. The composition can be formulated as described above with reference to a particular treatment method and can be packaged in any convenient manner.

The present invention will be further understood by reference to the following nonlimiting examples.

### EXAMPLES

### Example 1: Synthesis of β-GalNAc-Triantennary-PEG3-Azide Building Block

Tri-antennary Gal-NAc based hydroxyl PAMAM dendrimers were assessed for targeting and delivering drugs to hepatocytes in a site-specific manner. It has been shown that the surface GalNAc sugars create a multivalent binding effect to ASGPR, allowing the dendrimers to selectively target and internalize in hepatocytes in vivo in STAM model of nonalcoholic steatohepatitis.

Four different dendrimer-drug conjugates were synthesized and evaluated in this model: 1) D-GalNAc-Cy5 for targeting, 2) D-GalNAc-Telmisartan-ester (cleavable drug linker, Angiotensin 2 receptor blocker), 3) D-GalNAc-Telmisartan-amide (non-cleavable drug linker), and D-obeticholic acid (cleavable drug linker). It has been successfully demonstrated a precise loading of a combination of targeting ligand, imaging dye and therapeutic agents. The dendrimer can be further manipulated to attach a variety of combinations of therapeutic molecules. These results show that the GalNAc PAMAM dendrimers present an effective platform for the treatment of liver diseases.

### Methods

Synthesis scheme of P-GalNAc-triantennary-PEG3-Azide (AB3 building block) is shown in FIG. 1. Reagents and conditions: (i) scandium triflate, DCE, 3h, 80° C., (ii) propargyl bromide, toluene, sodium hydroxide, water, TBAB, (iii) pyridine, thionyl chloride, chloroform, 65° C., 2h; (iv) tetrabutylammonium hydrogen sulfate, 50% NaOH, 16h, rt; (v) (iii) CuSO₄.5H₂O, Na ascorbate, THF, water, 10 h; (vi) DMF, tetrabutylammonium iodide, NaN₃, 80° C., 5h; (vii) sodium methoxide, dry methanol, 30° C., 3h.

A triantenary building block was prepared where three molecules of beta-GALNAc-PEG3 azide are grafted on a propargylated pentaerythritol building block to yield AB₃ type orthogonal building block. Synthesis was started with the glycosylation reaction of β-D-GalNAc pentacetate (1, FIG. 1) with 2-[2-(2-azidoethoxy)ethoxy]ethan-1-ol (2) in the presence of scandium triflate in dichloroethane to yield peracetylated β-GalNAc-PEG3-azide (3). On the other hand, pentaerythritol 4 was selectively modified with 3 propargyl arms according to the literature method in the presence of sodium hydroxide and tetrabutylammonium bromide in DMSO to yield tripropargyl pentaerythritol (5). The remaining one hydroxyl group on the compound (5) was reacted with bis-chlorotetraethylene glycol (7) using sodium hydroxide and TBAB in DMSO to afford intermediate compound (8). During the next synthetic step, peracetylated β-GalNAc-PEG3-Azide was clicked with AB₃ building block (8) using conventional CuAAC click reaction conditions (Copper (II) sulphate pentahydrate and sodium ascorbate in THF:Water) to yield compound (9). The success of click reaction is confirmed by ¹H NMR, HRMS and HPLC. In the ¹H NMR, a signature sharp singlet of triazole at δ 7.9 ppm was observed. The other characteristic peaks are the acetate peaks between δ 2.0-1.74 ppm, GalNAc protons from δ 5.2-3.2 ppm and NH of GALNAC at δ7.78 ppm. In the next synthetic step, terminal chloride group of compound (9) was exchanged to azide by nucleophilic substitution in presence of sodium azide and tetrabutyl ammonium iodide in DMF to yield compound (10). The last step is the transesterification using zemplen conditions where the reaction was performed in methanol using sodium methoxide to afford deacetylated β-GalNAc-triantetennary-PEG3 azide (11) building block.

### Results

The successful completion of the reaction is confirmed by ¹H NMR where the peaks corresponding to O-acetates completely disappeared and all the sugar protons shifted upfield. The whole synthetic sequence was characterized using ¹H NMR, HPLC, and HRMS to confirm the desired compounds.

### Example 2: Synthesis and Characterization of Fluorescently Labeled Hepatocytes Targeting Dendrimer-Triantennary-β-GalNAc-CY5

Followed by the synthesis of targeting dendron (11), the synthesis of dendrimer-triantennary-β-GalNAc-CY5 was carried out to evaluate the selective hepatocyte targeting potential of this dendrimer using confocal microscopy and fluorescence spectroscopy.

### Methods

Dendrimer synthesis was initiated on PAMAM generation 4 hydroxyl terminated dendrimer 12 (FIG. 2) where partial esterification with 5 hexynoic acid (13) was achieved using Steglich esterification to yield compound (14). For this reaction EDC-HCl was used as coupling reagent with 4-dimethylaminopyridine (DMAP) and 12-14 hexyne arms were attached to the dendrimer. The structure of compound (14) was confirmed by ¹H NMR where the peak of esterified CH₂ at δ4.0 ppm was observed as well as the other multiplet corresponding to CH₂ from hexyne arm at δ1.7-1.6 ppm. For the exact determination of the loading of hexynoic arm, internal amide peak of the dendrimer at δ8.0-7.7 ppm was used as a reference point and using proton integration method, the number of attached arms were calculated. Once the hexynoic arms are introduced on dendrimer surface, the GalNAc dendron (3) (FIG. 1) was stitched with dendrimer (14) using copper catalyzed click (CuAAc) reaction to yield dendrimer (15). It was confirmed from the ¹H NMR that 5-6 arms of GalNAc dendron (11) are attached on the dendrimer. 5-6 arms of the GalNAc dendron were kept for targeting and 5-7 arms of hexyne were kept untouched to attach drugs or imaging agents. Introduction of 5-6 arms of dendron will result in 15-18 GalNAc units on the final structure.

For the loading calculations proton integration method was used. In the ¹H NMR, internal amide protons + 15 triazole protons from dendron and 5-6 protons for newly created triazole in between δ8.0-7.7 ppm, was observed. The NH peak from GalNAc was observed at δ7.6 ppm and N-acetyl singlet corresponding to 45 protons at δ1.78 ppm. When the ¹H NMR was recorded in D₂O, all the signals related to NH are exchanged with water and disappear and two different triazole peaks were clearly seen corresponding to 15 and 5 protons at δ8.0 & 7.8 ppm. All the GalNAc and dendrimers signal can be observed in between δ5.0-1.5 ppm. Once the targeting moiety is attached to dendrimer, the next step is to attach a fluorescent tag to this dendrimer. A near infra-red dye CY5 as a fluorescent tag. For the attachment of CY5 azide (16) with dendrimer (15) CuAAc reaction was employed to yield fluorescent-GalNAc dendrimer (17).

### Results

The click reaction successfully generated the CY5 labeled dendrimer where 2-3 molecules of the CY5 (a representative small molecule, useful as a diagnostic and predictive of results with small molecule drugs) are attached. The final dendrimer was characterized with ¹H NMR and CY5 loading was calculated using proton integration method. The peaks corresponding to CY5 appeared in between δ 7.5-6.2 ppm. The entire synthetic sequence was tracked using HPLC. The HPLC spectrum of G4-OH comes at 6.1 minutes. The HPLC spectrum shifted to hydrophobic side at 9.4 minutes when the hexyne arms were added to dendrimer and once again shifted towards the hydrophilic side when the water-soluble triantennary GalNAc dendron was conjugated to dendrimer and appears at 7.7 minutes. The addition of CY5 again shifted the peak towards right at 8.4 minutes. The final CY5 labeled dendrimer (17) was > 98% pure determined by HPLC.

### Example 3: Synthesis and Characterization of Dendrimer-GalNAc-Telmisartan Conjugates With Enzyme Cleavable and Non-Cleavable Linkers for NASH Treatment

After the successful completion of the fluorescent dendrimer, the next aim was to synthesize the dendrimer with targeting moiety and drug for liver disorders. NASH is well-recognized global health problem which leads to diseases like liver cirrhosis and hepatocellular carcinoma. Many different types of therapeutic molecules such as PPAR gamma, antioxidant agents and cytoprotective agents have been used with limited or no success.

### Methods

Currently, there is no approved drug for the treatment of NASH and other chronic liver diseases. Recently, telmisartan which is an angiotensin receptor blocker (ARB) and a partial agonist of peroxisome proliferator receptor, has shown promise in many animal models of NASH by increasing insulin sensitivity and inhibiting lipid accumulation in the liver. Despite the good results, its clinical translation is hampered by the dose related cytotoxicity and other side effects like hypotension.

To overcome these challenges, a highly specific targeted drug delivery nanoplatform is required to deliver loads of drug to the desired organ or tissue. For this purpose, Telmisartan was attached to Dendrimer-GalNAc (15) using two different linkages i.e Telmisartan ester (FIG. 3) and Telmisartan amide (FIG. 4). For the synthesis of Telmisartan-ester PEG4 azide (19) telmisartan was reacted with 2-(2-(2-(2-azidoethoxy)ethoxy)ethoxy)ethan-l-ol (2) in the presence of DCC and DMAP in dry DCM. The Telmisartan ester-PEG4 azide was achieved in quantitative yield.

For the synthesis of Dendrimer-Triantenary-β-GlcNAc-azide-Telmisartan ester conjugate, the product was confirmed using ¹H NMR and LCMS. In the ¹H NMR, the aromatic protons (14) appears in between δ 7.8-7.1 ppm, the benzylic CH₂ appears at δ 5.6 ppm, the CH₂ next to CH₃ is at δ 1.85 ppm and a triplet corresponding to CH₃ is at δ 1.0 ppm. Once the successful formation of Telmisartan azide is confirmed, it was clicked with the GalNAc dendrimer (15) harboring 6-7 arms of hexyne linker, using CuAAC click reaction. The click reaction was successful to create the dendrimer-triantennary GalNAc(4-5)-telmisartan (7-8) (20). The product confirmation was once again achieved by ¹H NMR. The dendrimer internal amide peaks, triazole peaks, aromatic protons from telmisartan and NH-corresponding to GalNAc appears in between δ 8.0-7.0 ppm. The benzylic CH₂ appears at δ 5.6 ppm. Others important peaks to watch for, are N-acetyl peak at δ 1.7 ppm and CH₃ peak at δ 0.9 ppm. The proton integration method was used to calculate the drug loading. It was confirmed that 7-9 molecules of telmisartan ester are attached with the dendrimer. The weight % loading of telmisartan was around 14% considering 8 molecules of telmisartan were attached. The telmisartan is a very hydrophobic drug but the conjugate is highly water soluble and the solubility is around 60 mg/ml. The purity of the final conjugate is >96%

After the successful completion of Dendrimer telmisartan with enzyme sensitive ester linkage, a dendrimer-telmisartan amide conjugate was synthesized which should be more stable under physiological conditions (FIG. 4). To achieve this, a linker azide to telmisartan was first introduced by coupling it with 2-(2-(2-(2-azidoethoxy)ethoxy)ethoxy)ethan-1-amine (21) using HATU and DIPEA to afford telmisartan-PEG3-amide azide (22). The compound was characterized using ¹H NMR and LCMS. Once the azide-functionalized telmisartan is formed, it was conjugated with dendrimer-GalNAc (15) using CuAAc to afford dendrimer-GalNAc (4-5)-telmisartan amide (6-7) (23).

For the synthesis of Dendrimer-Triantenary-β-GlcNAc-azide-Telmisartan amide conjugate, the product confirmation was achieved by ¹H NMR. Once the drug molecules are attached, all the signature peaks from the drug in the final compound were observed. The dendrimer internal amide peaks, triazole peaks, aromatic protons from telmisartan and NH-corresponding to GalNAc appears in between δ 8.2-7.1 ppm. The benzylic CH₂ appears at δ 5.6 ppm. Others important peaks are N-acetyl peak at δ 1.8 ppm and CH₃ peak at δ 1.0 ppm. When the ¹H NMR was recorded in D₂O, the internal amide peaks corresponding to the dendrimer were exchanged and disappeared and singlet corresponding to triazole appears at δ 8.0 ppm. The proton integration method was used to calculate the drug loading.

### Results

It was confirmed that 6 molecules of telmisartan amide are attached to the dendrimer. The weight % loading of telmisartan was around 11% considering 6 molecules of telmisartan were attached. The final conjugate is highly water soluble and the solubility is around 60-70 mg/ml.

The progress of the entire synthetic process was tracked by HPLC. The retention time of the GalNAc triantennary conjugate (15) is 7.8 minutes but once the addition of hydrophobic telmisartan azide takes place the retention time of the final conjugate (23) is shifted towards the hydrophobic side and comes at 10.4 minutes. The purity of the final conjugate is >95%.

### Example 4: Binding Affinity of Telmisartan and Telmisartan Linkers Against Human Angiotensin II AT1 Receptor

### Methods

The binding affinities of the telmisartan, telmisartan-ester-PEG4 azide and telmisartan-PEG3-amide azide were evaluated using human angiotensin II AT1 receptor (antagonist radioligand) binding assay (Table 1). Cell membrane homogenates (8 µg protein) are incubated for 120 min at 37° C. with 0.05 nM [125I][Sar1-Ile8] angiotensin-II in the absence or presence of the test compounds in a buffer containing 50 mM Tris-HCl (pH 7.4), 5 mM MgCl₂, 1 mM EDTA and 0.1% BSA. Nonspecific binding is determined in the presence of 10 µM angiotensin II. Following incubation, the samples solution are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard).

### Results

The results are expressed as a percent inhibition of the control radioligand specific binding. Saralasin is used as the standard reference compound, which is tested in each experiment at several concentrations to obtain a competition curve from which its IC50 is calculated.

Sample preparation: Telmisartan, telmisartan ester linker and telmisartan amide linker were dissolved in aqueous DMSO to form solution at free drug (telmisartan) concentration of 10 mM. Each sample solution was further diluted to 10 µM, 3.33 µM, 1.11 µM, 0.37 µM, 0.123 µM, 41.2 nM, 13.7 nM, 4.57 nM, 1.52 nM, 0.508 nM and 0.169 nM in DMSO respectively for binding study.

The binding affinity of modified drug linkers retained the values in nanomolar range (Table 1).

TABLE 1

Binding affinity of telmisartan, telmisartan-ester-PEG4 azide and telmisartan-PEG3-amide azide

| Compound No. | Compounds Name | Gene Symbol | Kd (nM) |
|---|---|---|---|
| 1 | Telmisartan | Angiotensin II AT1 | 3 |
| 2 | Telmisartan ester linker | Angiotensin II AT1 | 28.5 |
| 3 | Telmisartan amide linker | Angiotensin II AT1 | 74.2 |

### Example 5: Dendrimer-Telmisartan-Ester and Dendrimer -Telmisartan-Amide Release Study

### Methods

The drug release profile of both the conjugates was evaluated under plasma (pH7.4, PBS) and intracellular conditions (pH5.5, esterase).

### Results

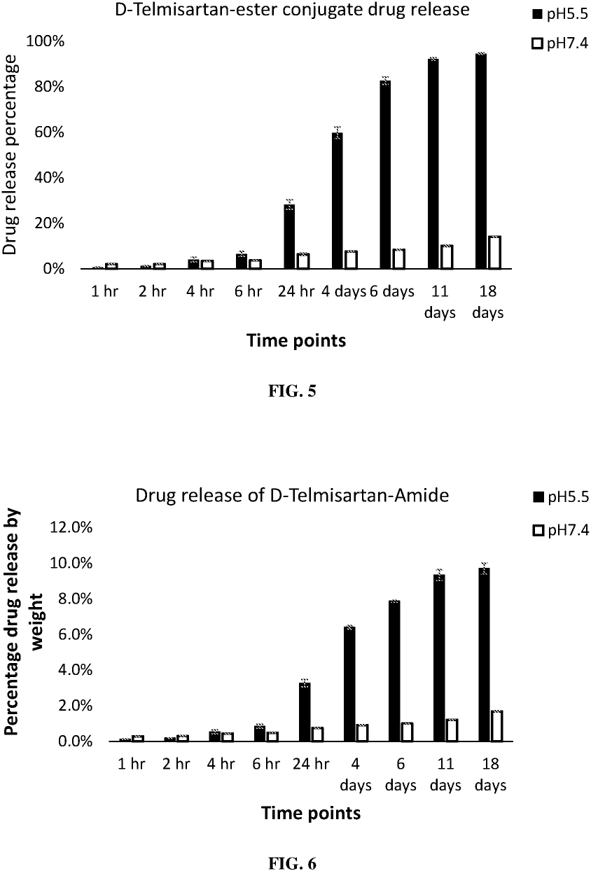

The results indicate that at plasma physiological conditions the dendrimer-ester linked construct is very stable and only 14% drug release has been observed in 18 days but under intracellular conditions 94% of the drug is released in 18 days (FIG. 5). However, in the Dendrimer-telmisartan amide conjugate release experiment, less than 2% drug was released in 18 days at plasma physiological conditions and less than 10% drug was released under intracellular conditions in 18 days (FIG. 6). The amide-linked drug conjugate is more stable than the ester linked dendrimer conjugate under physiological conditions.

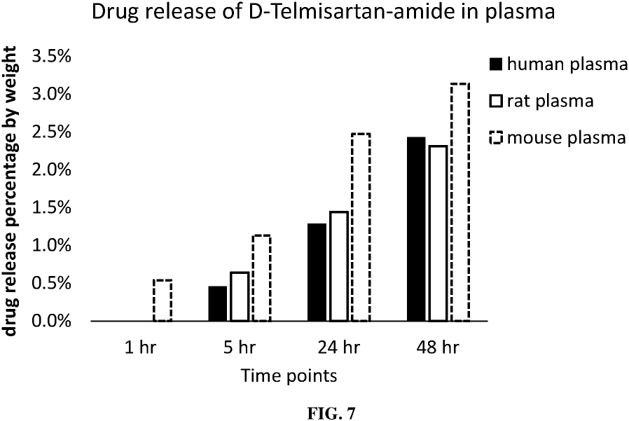

The stability of the D-telmisartan amide conjugate was also evaluated in human, mouse and rat plasma at 37° C. and only 3% of drug is released over the period of 2 days (FIG. 7).

### Example 6: Synthesis and Characterization of Dendrimer-GalNAc-Obiticholic Acid Conjugate for NASH Treatment

A very potent drug, obeticholic acid, which is a semisynthetic bile acid analogue and is the most active physiological ligand for farnesoid X receptor which have shown promising results in NASH but has dose related toxicity issues, was utilized. For the conjugation of obeticholic to dendrimer-GalNAc-hexynoic acid (15), the carboxylic acid functional handle of obeticholic acid (24) was selectively esterified with PEG4azide linker (25) using EDC, DMAP coupling reaction (FIG. 8). The obeticholic acid linker azide (26) was characterized using ¹H NMR, HRMS and HPLC. The azide terminated obeticholic acid was conjugated successfully with the dendrimer-GalNAc-hexyne (15) using click reaction to afford dendrimer-GalNAc(4-5)-Obeticholic acid conjugate (6-7) (27). The dendrimer and the intermediates were characterized thoroughly using ¹H NMR (FIG. 8) and HPLC. The drug loading of the dendrimer was calculated with proton integration method where dendrimer internal amide protons were used as reference peak. The methyl peak belongs to the obeticholic acid at δ 0.6 ppm helped to calculate exact drug loading. 6 molecules of the obeticholic acid were attached to the dendrimer and the drug loading is approximately 9.5%. The purity of the final conjugate is more than 99% and the solubility range is ~100 mg/mL.

### Example 7: In Vivo Efficacy Study of Triantennary-Galnac Modified Hydroxyl Dendrimers in Mouse Models of Non-Alcoholic Steatohepatitis

### Methods

### Mice and NASH Model

Pathogen-free 14 day-pregnant C57BL/6 mice were obtained from Japan SLC, Inc.(Japan); NASH was established in male mice by a single subcutaneous injection of 200 µg streptozotocin (STZ, Sigma, USA) 2 days after birth and feeding with a high fat diet (CLEA Japan Inc., Japan) ad libitum after 4 weeks of age (day 28 ± 2). NASH mice were randomized into 8 groups of 8 mice and 2 groups of 4 mice at 6 weeks of age (day 42 ± 2) the day before the start of treatment based on their body weight. Littermate control mice without STZ priming (n=8) were fed with normal diet ad libitum and set up for control purpose (see below for details on grouping). If an animal showed >25% body weight loss within a week or >20% body weight loss compared to previous day, the animal would be euthanized ahead of study termination. If it showed a moribundity sign such as prone position, the animal would be euthanized ahead of study termination. Samples would not be collected from euthanized animals. Individual body weight was measured daily during the treatment period. Survival, clinical signs and behavior of mice were monitored daily.

### Grouping

Group 1 (Normal): Eight normal mice were fed with normal diet ad libitum without any treatment and sacrificed at 9 weeks of age;
Group 2 (Vehicle): Eight NASH mice were intraperitoneally administered vehicle (saline) in a volume of 10 mL/kg every other day from 6 to 9 weeks of age;
Group 3 (Telmisartan): Eight NASH mice were orally administered pure water supplemented with Telmisartan at a dose of 10 mg/kg once daily from 6 to 9 weeks of age;
Group 4 (Obeticholic acid, or “OCA”): Eight NASH mice were orally administered 1% methlycelluose supplemented with OCA at a dose of 30 mg/kg once daily from 6 to 9 weeks of age;
Group 5 (Dendrimer-Triantenary-β-GlcNAc-azide-Telmisartan amide conjugate, or “D-Tel” high): Eight NASH mice were intraperitoneally administered vehicle supplemented with D-Tel at a dose of 90 mg/kg every other day from 6 to 9 weeks of age;
Group 6 (D-Tel low): Eight NASH mice were intraperitoneally administered vehicle supplemented with D-Tel at a dose of 18 mg/kg every other day from 6 to 9 weeks of age;
Group 7 (Dendrimer-Triantenary-β-GlcNAc-azide-Telmisartan ester conjugate, or “D-TelB” high): Eight NASH mice were intraperitoneally administered vehicle supplemented with D-TelB at a dose of 90 mg/kg every other day from 6 to 9 weeks of age;
Group 8 (D-OCA high): Eight NASH mice were intraperitoneally administered vehicle supplemented with D-OCA at a dose of 315 mg/kg (equivalent to about 30 mg/kg of OCA conjugated to the dendrimers at this dosage) every other day from 6 to 9 weeks of age.
Group 9 (D-OCA low): Eight NASH mice were intraperitoneally administered vehicle supplemented with D-OCA at a dose of 63 mg/kg (equivalent to about 6 mg/kg of OCA conjugated to the dendrimers at this dosage) every other day from 6 to 9 weeks of age.
Group 10 (D-Cy5-6 wks): Four NASH mice were intraperitoneally administered vehicle supplemented with D-Cy5 at a dose of 50 mg/kg single shot at 6 weeks of age;
Group 11 (D-Cy5-9 wks): Four NASH mice were intraperitoneally administered vehicle supplemented with D-Cy5 at a dose of 50 mg/kg single shot at 9 weeks of age.

Mice in group 10 and 11 were sacrificed at 6 and 9 weeks of age 48 hours after the administration. Mice in group 1 - 9 were sacrificed at 9 weeks of age for the following assay, group 10 and 11 were sacrificed at 6 and 9 weeks of age for the following assays. Individual liver weight were measured and liver-to-body weight ratio were calculated.

### Biochemical Assays (group 1 - 9)

Non-fasting serum ALT levels were quantified by FUJI DRI CHEM (Fujifilm, Japan). Liver triglyceride levels were quantified by Triglyceride E-test kit (FUJIFUILM Wako Pure Chemical Corporation, Japan).

### Histological Analyses for Liver Sections (group 1 - 9)

HE staining and estimation of NAFLD Activity score was carried out using routine methods. Sirius-red staining and estimation of the percentage of fibrosis area was also calculated.

### Sample Collection and Fixation

After completion of the in-life portion of the study, the following samples were collected for further analyses or shipping. Animals in group 10-11 were anesthetized with isoflurane and perfused with saline (followed by 4% neutral buffered formalin, NBF, pH 7.4) through left ventricle for 20-30 min. Dissect the animal and collect the tissue samples (right and left kidneys, liver) in sequential order. Sample thickness was less than approximately 5 mm to ensure proper fixation, with a flat surface across the area of interest. Put the samples into 4% NBF for fixation immediately. Fix the samples in 4% NBF overnight at room temperature.

### Results

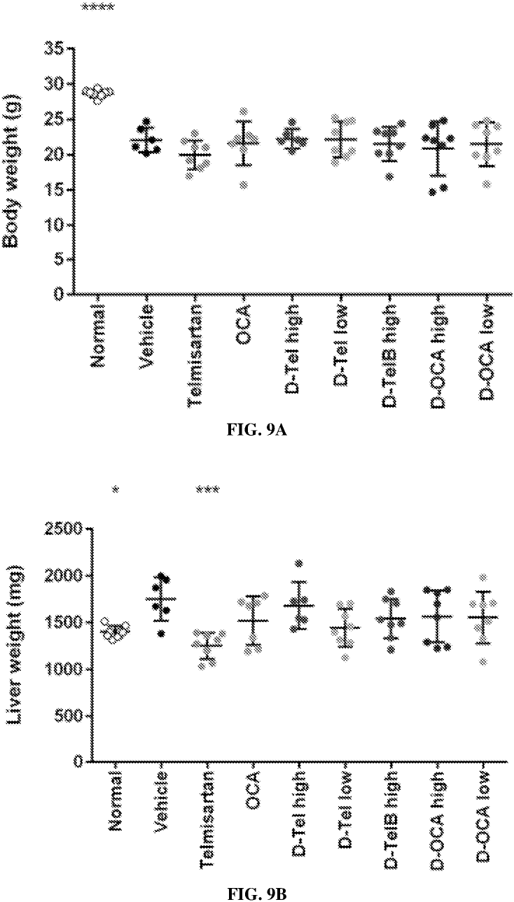

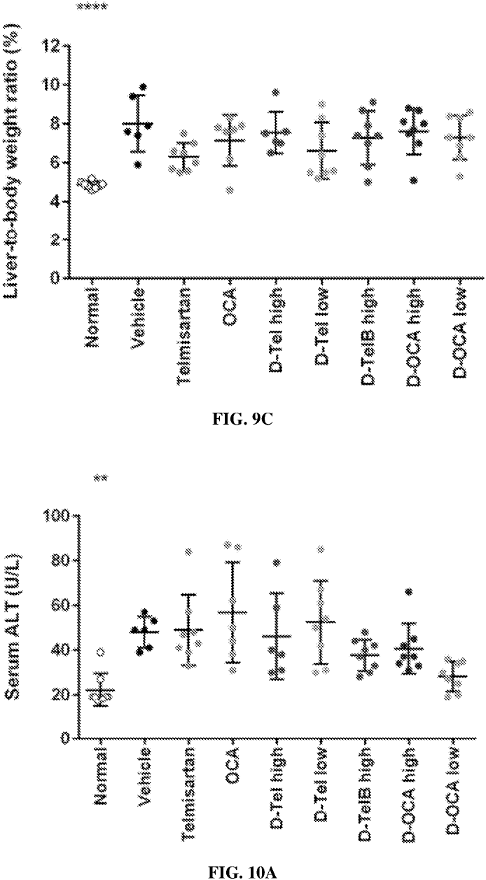

Individual body weight was measured throughout the treatment period. Experimental NASH mice without (vehicle) or with treatment maintained a similar body weight throughout. Normal mice weighed about 26-27 grams throughout the experiment which was more than all NASH mice but the NASH mice from all treatment groups showed a similar body weight throughout the treatment period. At nine weeks, i.e., three weeks after treatment began, body weight from different treatment groups did not showed significant difference (FIG. 9A). All treatment groups except the groups treated with free telmisartan showed similar liver weight at nice weeks. NASH mice treated with free telmisartan had reduce liver weight compared to vehicle-treated NASH mice (FIG. 9B). FIG. 9C shows liver-to-body weight ration of all experimental groups.

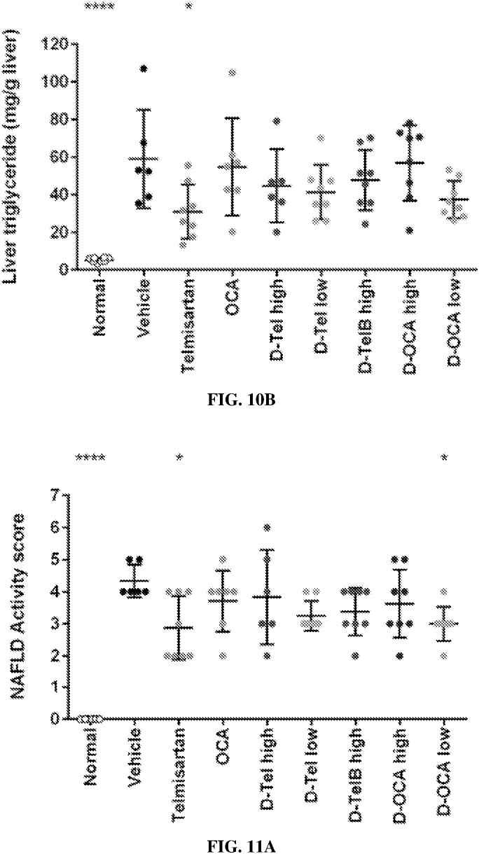

Biochemical assaying measuring non-fasting serum ALT levels and liver triglyceride levels were carried out at nine weeks of age (FIGS. 10A and 10B).

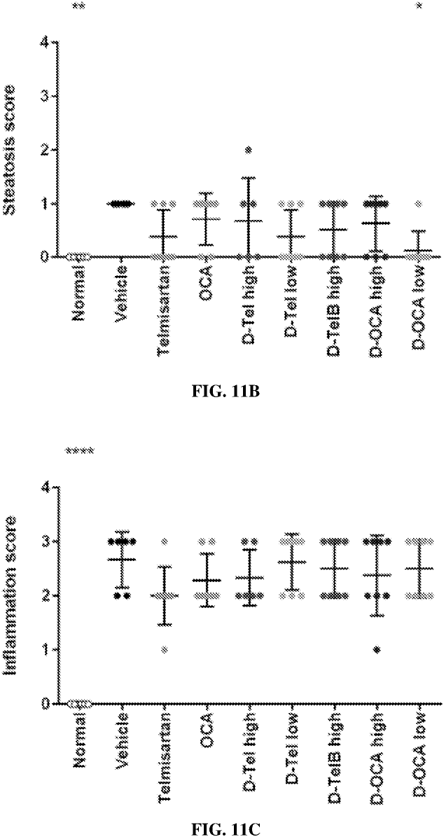

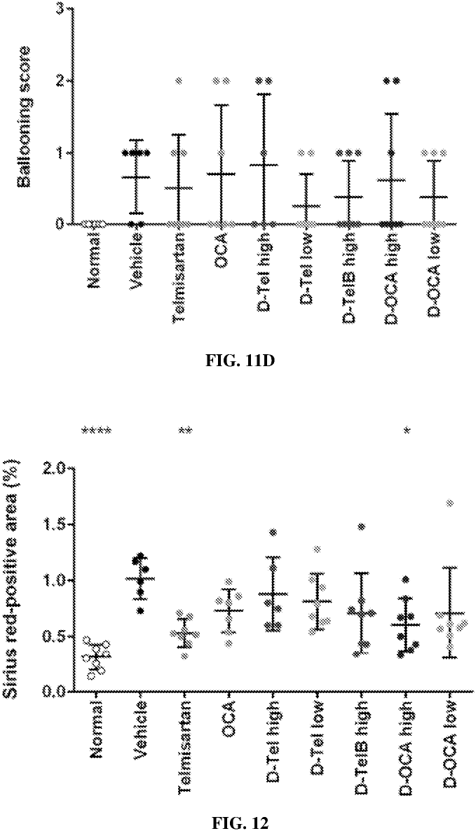

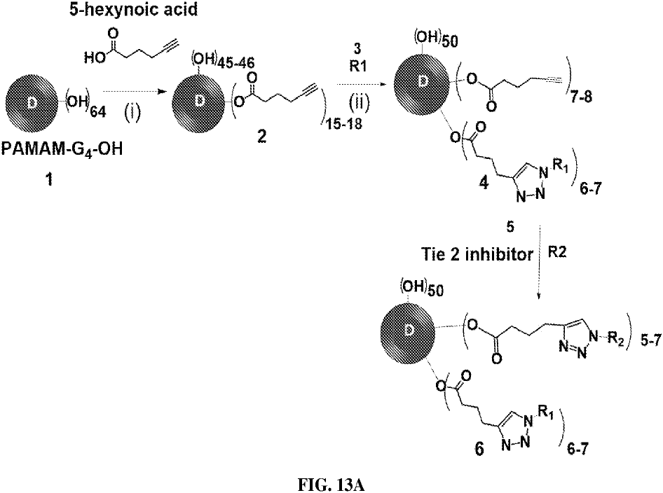

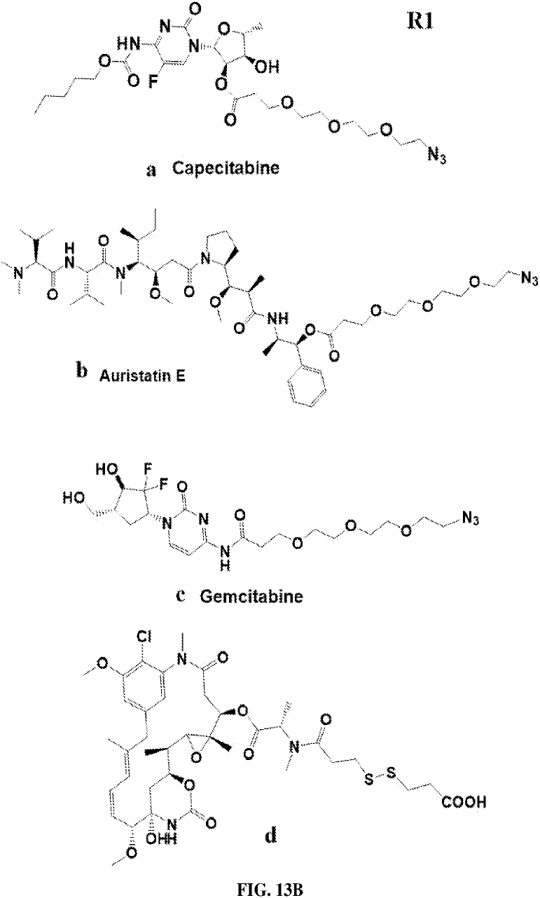

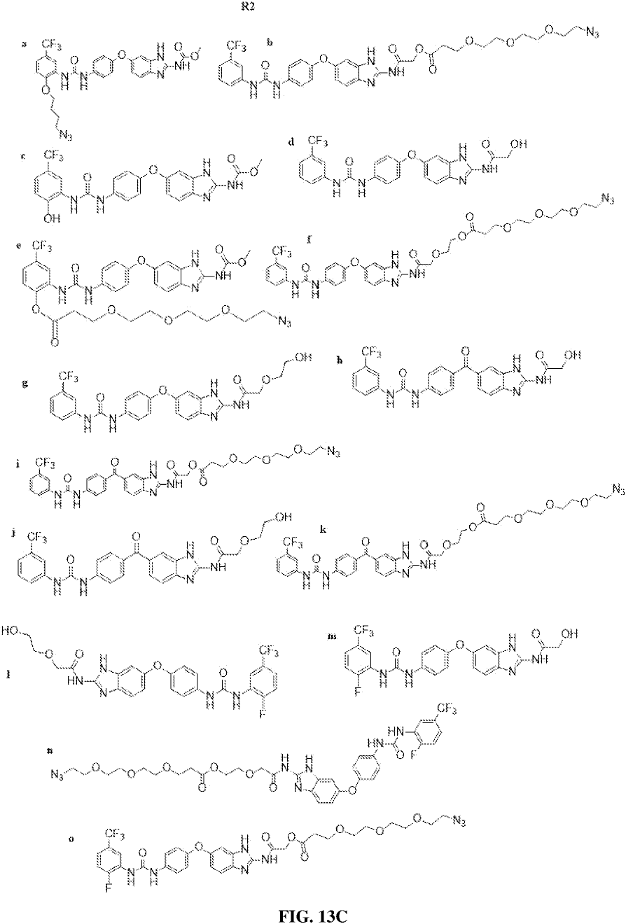

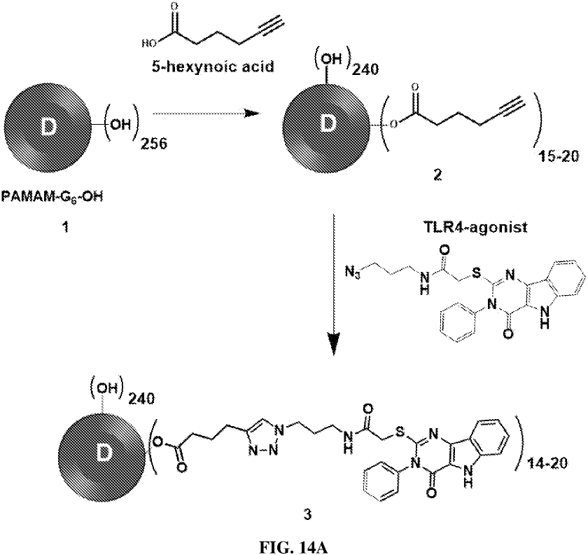

Histopathologic analysis was performed on the livers of normal mice, and the NASH mice from all treatment groups when sacrificed at 9 weeks of age. Nonalcoholic fatty liver disease (NAFLD) activity score, steatosis score, inflammation score, and ballooning score are shown in FIGS. 11A-11D. Sirius red staining was used to assess the extent of fibrosis in livers of of normal mice, and the NASH mice from all treatment groups (FIG. 12).

Obeticholic acid (OCA) is a potent and selective famesoid X receptor agonist (FXRa). Immunohistochemistry analysis of liver tissues demonstrates that dendrimer-triantenary-β-GlcNAc-azide-obeticholic acid ester conjugate (D-OCA) decreases NAFLD score, liver fibrosis and hepatocyte ballooning significantly compared to free OCA and vehicle control (p < 0.05, n=8). Low dose D-OCA treatment showed significant reduction in stenosis score whereas high dose D-OCA treatment showed improved reduction in fibrosis score compared to free OCA group. Biochemical analysis also suggests that hepatocyte targeted hydroxyl dendrimer conjugated obeticholic acid treatment improved liver function.

In summary, a hepatocyte targeted hydroxyl dendrimer therapeutic has been established to enable selective targeting of FXRa to hepatocytes through the asialoglycoprotein receptor (ASGP-R) mediated uptake after systemic administration, enhancing the drug efficacy and reducing the dose and off-site toxicity. Selective targeting of FXRa to hepatocytes improves functional outcomes in a NASH model. This targeted approach significantly reduces systemic off-target toxicity observed with current FXRa compounds. Previous studies with hydroxyl terminated dendrimers demonstrated a sustained localization within the target cells for up to 1 month. Overall, the hepatocyte targeted hydroxyl dendrimer approach provides a platform for developing a wide range of drugs to treat liver diseases.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1-36. (canceled)

37. A method of treating one or more symptoms of a liver disease or disorder in a subject in need thereof, the method comprising:
administering to the subject a composition comprising a hydroxyl-terminated dendrimer conjugated to a triantennary N-acetylgalactosamine (GalNAc) and a therapeutic agent,
wherein the composition is administered in an amount effective to treat the one or more symptoms of the liver disease or disorder in the subject.

38. The method of claim 37, wherein the dendrimer is conjugated to the triantennary GalNAc through an ether or amide linkage.

39. The method of claim 37, wherein the dendrimer is conjugated to the therapeutic agent through an ether or amide linkage.

40. The method of claim 37, wherein the triantennary GalNAc is conjugated to the dendrimer through a first linker, and the therapeutic agent is conjugated to the dendrimer through a second linker.

41. The method of claim 40, wherein the first and second linkers are attached to different terminal groups on the dendrimer to form an ether linkage between each linker and the dendrimer.

42. The method of claim 40, wherein at least one of the first and second linkers comprise polyethylene glycol.

43. The method of claim 37, wherein the dendrimer comprises more than one triantennary GalNAc.

44. The method of claim 37, wherein the therapeutic agent is selected from the group consisting of angiotensin II receptor blockers, Farnesoid X receptor agonists, death receptor 5 agonists, sodium-glucose cotransporter type-2 inhibitors, lysophosphatidic acid 1 receptor antagonists, endothelin-A receptor antagonist, PPARδ agonists, AT1 receptor antagonists, CCR5/CCR2 antagonists, anti-fibrotic agents, anti-inflammatory agents, anti-oxidant agents, STING agonists, CSF1R inhibitors, PARP inhibitors, VEGFR tyrosine kinase inhibitors, EGFR tyrosine kinase inhibitors, MEK inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, and chemotherapeutics.

45. The method of claim 44, wherein the therapeutic agent is an antifibrotic agent.

46. The method of claim 44, wherein the therapeutic agent is a death receptor 5 agonist selected from the group consisting of an agonistic antibody, a small molecule DR5 agonist, an avimer, an Fc fusion protein, apo2L/TRAIL, human TRAIL ligand, and recombinant soluble TRAIL.

47. The method of claim 37, wherein the dendrimer is a generation 4, generation 5, generation 6, generation 7, or generation 8 poly(amidoamine) dendrimer.

48. The method of claim 37, wherein the liver disease or disorder is selected from the group consisting of nonalcoholic fatty liver disease, non-alcoholic steatohepatitis, drug-induced liver failure, hepatitis, liver fibrosis, liver cirrhosis, and hepatocellular carcinoma.

49. The method of claim 37, wherein the liver disease or disorder is a cholangiopathy, a metabolic disease, or a proliferative disease.

50. A hydroxyl-terminated dendrimer comprising a triantennary N-acetylgalactosamine (GalNAc) and a therapeutic agent, wherein the triantennary GalNAc is conjugated to the dendrimer through a first linker, wherein the therapeutic agent is conjugated to the dendrimer through a second linker, and wherein the first and second linkers are attached to different terminal groups on the dendrimer to form an ether linkage between each linker and the dendrimer.

51. The dendrimer of claim 50, wherein at least one of the first and second linkers comprise polyethylene glycol.

52. The dendrimer of claim 50, wherein at least one of the first and second linkers comprise an amide bond.

53. The dendrimer of claim 50, wherein the therapeutic agent is a small molecule, a peptide, a protein, a nucleic acid, or an oligonucleotide.

54. The dendrimer of claim 50, wherein the therapeutic agent is an antifibrotic agent.

55. The dendrimer of claim 54, wherein the therapeutic agent is a death receptor 5 agonist selected from the group consisting of an agonistic antibody, a small molecule DR5 agonist, an avimer, an Fc fusion protein, apo2L/TRAIL, human TRAIL ligand, and recombinant soluble TRAIL.

56. A method of making dendrimers having one or more triantennary GalNAc molecules, the method comprising:
(a) preparing a hyper monomer AB₃, wherein the preparing comprises performing propargylation of the hyper monomer at three reactive groups;
(b) conjugating one azide group onto a N-acetylgalactosamine (GalNAc) molecule to produce a GalNAc-azide building block;
(c) mixing the hyper monomer AB₃ from step (a) and the GalNAc-azide building block from step (b) and performing a copper (I)-catalyzed reaction to yield a triantennary GalNAc; and
(d) conjugating the triantennary GalNAc onto a reactive terminal group of a dendrimer.
